(19) United States
(12) Patent Application Publication
(10) Pub. No.: US 2026/0256962 A1
(43) Pub. Date: Sep. 3, 2026

(54) PRODRUGS FOR COMPOUNDS SPECIFIC TO GRANZYME B AND USES THEREOF

(71) Applicant: CYTOSITE BIOPHARMA INC., Sudbury, MA (US)

(72) Inventors: Geoffrey Malcolm BILCER, Shorewood, MN (US); Hui XIONG, Indianapolis, IN (US); Carey HORCHLER, Indianapolis, IN (US); Mark A. CASTANARES, Indianapolis, IN (US); Brian LIEBERMAN, Indianapolis, IN (US); Wei ZHANG, Indianapolis, IN (US); Xuan HUANG, Indianapolis, IN (US); Juntian ZHANG, Indianapolis, IN (US)

(73) Assignee: CYTOSITE BIOPHARMA INC., Sudbury, MA (US)

(21) Appl. No.: 18/718,416

(22) PCT Filed: Dec. 7, 2022
(86) PCT No.: PCT/US2022/081125

§ 371 Date: Jun. 10, 2024

Related U.S. Application Data
(60) Provisional application No. 63/287,473, filed on Dec. 8, 2021.

(51) Int. Cl. A61K 51/08; A61K 45/06; A61K 51/04

(52) U.S. Cl. CPC A61K 51/088; A61K 45/06; A61K 51/0482; A61K 51/0497

(57) ABSTRACT

Compounds of Formula (I) and Formula (II), which are capable of binding to granzyme B. Also provided herein are pharmaceutical compositions comprising such for use in, for example, imaging Granzyme B and/or treating immunoregulatory abnormalities.

### CROSS-REFERENCE TO RELATED APPLICATIONS

This application claims the benefit of U.S. Provisional Application No. 63/287,473, filed Dec. 8, 2021, the content of which is incorporated by reference herein in its entirety.

### TECHNICAL FIELD

This disclosure relates to prodrug compounds that can convert to active forms useful for imaging techniques in vivo, and more particularly to prodrug compounds that can convert to active compounds specific to Granzyme B and useful for imaging Granzyme B using medical imaging, including positron emission tomography.

### BACKGROUND

Granzyme B is a serine-protease most commonly found in the granules of natural killer cells and cytotoxic T cells. Granzyme B is released along with the pore-forming protein perforin at the immunological-synapse formed between T-cells and their targets. A portion of the released Granzyme B then enters cancer cells, primarily through perforin-pores, where it activates multiple substrates leading to activation of the caspase cascade. As a downstream effector of tumoral cytotoxic T cells, granzyme B has been used as an early biomarker for tumors responding to immunotherapy.

There is a need to develop new compounds that act as effective Granzyme B imaging agents, and therapies for treating immunoregulatory abnormality such as cancer.

### SUMMARY

The present disclosure is based, at least in part, on the development of prodrug compounds that can convert to active granzyme B(GZB)-binding compounds, e.g., in vivo. Such prodrug compounds (i.e., pro-form of GZB-binding compounds) exhibit superior features, such as production of single isomer, synthesis with reliable stereochemical outcome, facile liberation of the active GZB-binding compounds in vivo, or a combination thereof. Such pro-forms of granzyme B (GZB)-binding compounds can be used in GZB imaging (e.g., in vivo), the results of which can be relied upon for therapeutic and diagnostic purposes, for example, for identifying suitable patients for treatment and/or for monitoring treatment efficacy.

Accordingly, in one aspect, the present disclosure features a compound, or a pharmaceutically acceptable salt thereof, of formula (I):

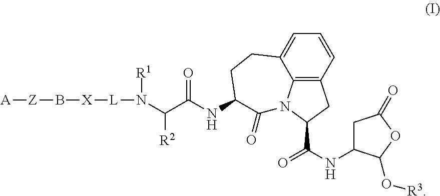

In formula (I),
A is a chelating moiety (e.g., those disclosed herein);
B is selected from the group consisting of aryl, heteroaryl, cycloalkyl, and heterocyclyl;
X is selected from the group consisting of —CH₂C(NH)—, —CH₂C(O)—, —CH₂C(S)—, —NHC(NH)—, —NHC(O)—, —NHC(S)—, —OC(NH)—, —OC(O)—, and —OC(S)—;
Z is —CH₂—, —CH₂C(NH)—, —CH₂C(O)—, —CH₂C(S)—, —NHC(NH)—, —NHC(O)—, —NHC(S)—, —OC(NH)—, —OC(O)—, and —OC(S)—;
L is a peptide linker having 1-6 amino acid residues, inclusive;
R¹ is H or C₁₋₆ alkyl, optionally wherein R¹ is H or methyl;
R² is C₁₋₆ alkyl or C₃₋₆ cycloalkyl; and
R³ is C₁₋₆ alkyl.

In some examples, B can be a 6-membered ring. In particular examples, B can be a 6-membered aryl ring. In other examples, B can be a 6-membered heterocyclyl.

In some examples, X can be —CH₂C(O)—. In other examples, X can be —NHC(S)—.

In some examples, Z may be —CH₂— (e.g., when B is a piperidyl ring). Alternatively, Z may be —CH₂C(O)— (e.g., when B is a piperazyl ring).

In some embodiments, R¹ is H and R² is C₄ alkyl, for example, as in compounds of formula (Ia):

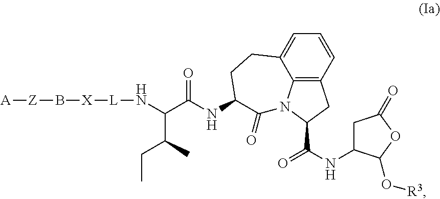

in which variables A, B, X, Z, L, and R³ are as defined herein.

In further embodiments, X is —CH₂C(O)—, for example, as in compounds of formula (Ib)

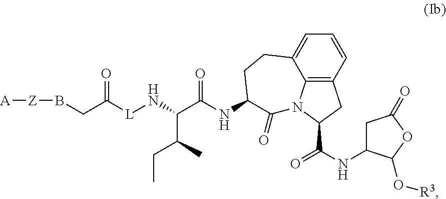

in which A, B, Z, L, and R³ are as defined herein.

In some of the above embodiments, R³ is methyl, for example, as in formula (Ic):

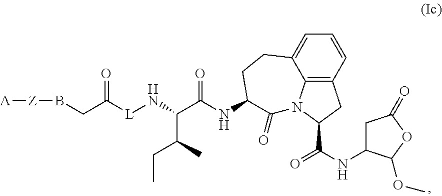

in which variable A, B, Z, and L each are as defined herein.

In some of the above embodiments, B is piperidyl, and Z is —CH₂—, for example, as in formula (Ic-A):

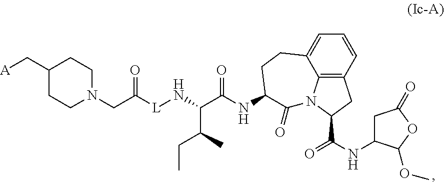

in which variables A and L each are as defined herein.

In some embodiments, the compound has defined stereochemistry as in formula (Ic-Aa):

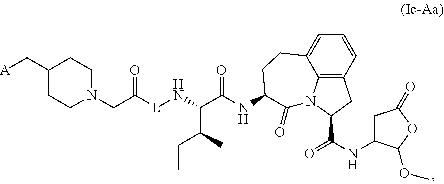

in which variables A and L each are as defined herein.

In particular examples, the compound has defined stereochemistry as in formula (Ic-Ab):

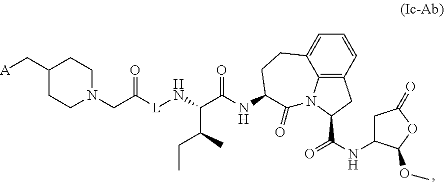

in which variables A and L each are as defined herein.

Exemplary Compounds of Formula (Ic-A) include Compounds 1-17.

In some of the above embodiments, B is piperazyl, and Z is —CH₂C(O)—, for example, as in Formula (Ic-B):

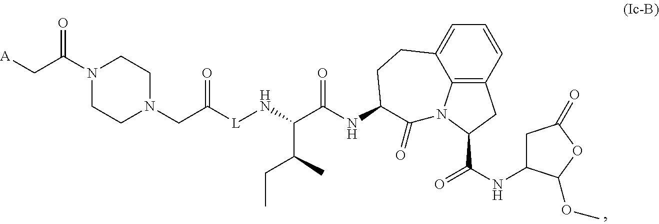

in which variables A and L are each as defined herein.

In some embodiments, the compound has defined stereochemistry as in formula (Ic-Ba):

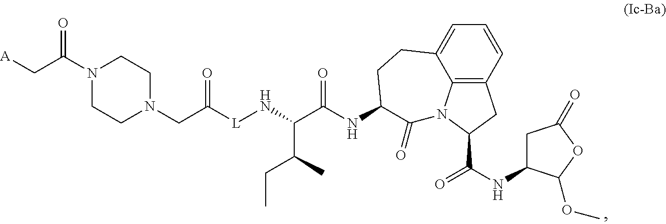

in which variables A and L are each as defined herein.

In particular examples, the compound has defined stereochemistry as in formula (Ic-Bb):

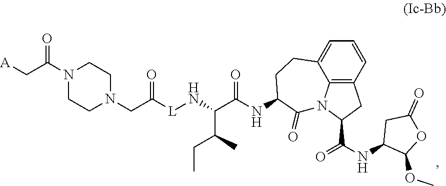

in which variables A and L are each as defined herein.

Exemplary Compounds of Formula (Ic-B) include Compounds 18-28.

In some embodiments, B is phenyl, and Z is —CH₂—, for example, as in Formula (Ic-C):

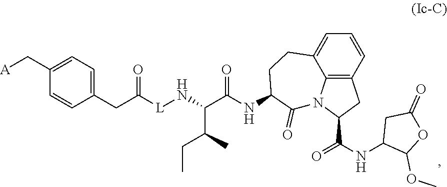

in which variables A and L are each as defined herein.

In some embodiments, the compound has defined stereochemistry as in formula (Ic-Ca):

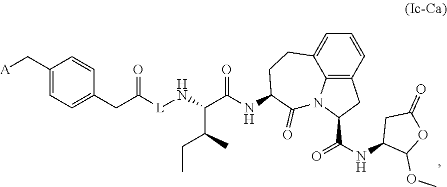

in which variables A and L are each as defined herein.

In particular examples, the compound has defined stereochemistry as in formula (Ic-Cb):

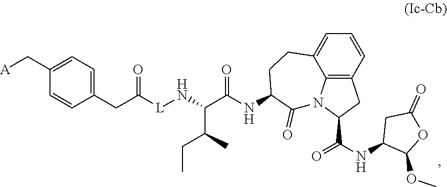

in which variables A and L are each as defined herein.

Exemplary Compounds of Formula (Ic-C) include Compounds 29-46.

In some embodiments, B is phenyl, R¹ is H, R² is C₄ alkyl, and R³ is methyl, as in formula (Id):

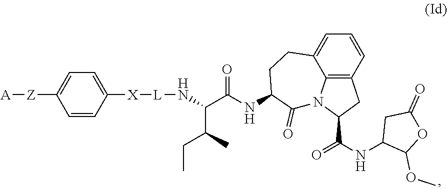

in which variables A, Z, X, and L each are defined herein.

In some embodiments, A is NOTA, and Z is —CH₂—, as in formula (Id-A):

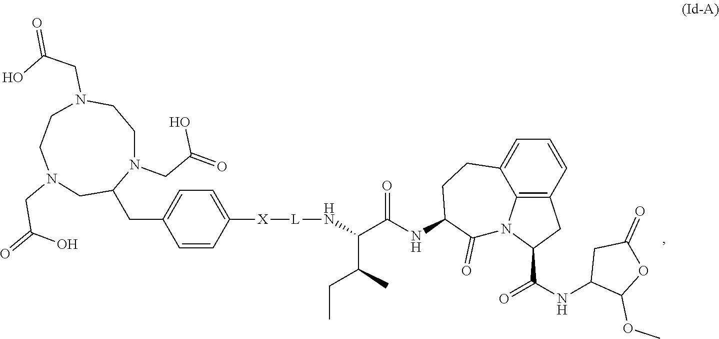

in which variables X, and L each are defined herein.

In some embodiments, the compound has defined stereochemistry as in formula (Id-Aa):

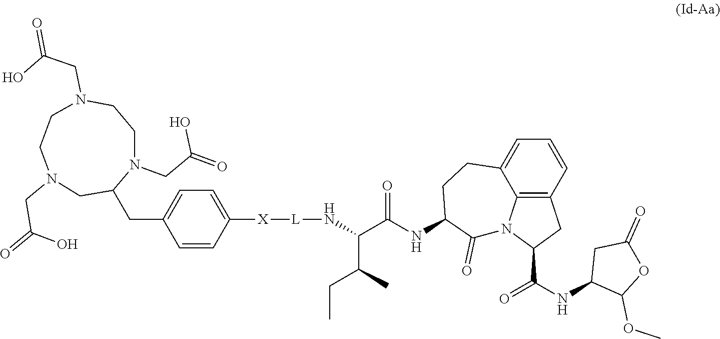

in which X and L are as define herein.

In particular examples, the compound has defined stereochemistry as in formula (Id-Ab):

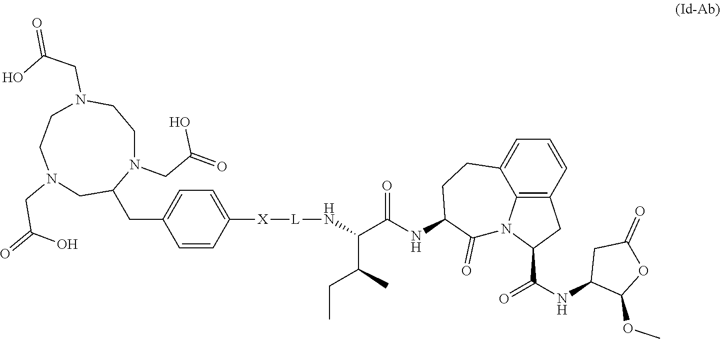

in which X and L are as defined herein.

Exemplary Compounds of Formula (Id-A) include Compound 47-49.

In another aspect, the present disclosure features a compound, or a pharmaceutically acceptable salt thereof, of formula (II):

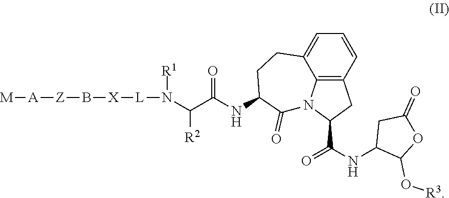

The variables of formula (II) may be described as:
M is a metal or a metal linked to a radioisotope;
A is a chelating moiety chelating the metal;
B is selected from the group consisting of aryl, heteroaryl, cycloalkyl, and heterocyclyl; optionally wherein B is a 6-membered ring;
X is selected from the group consisting of —CH₂C(NH)—, —CH₂C(O)—, —CH₂C(S)—, —NHC(NH)—, —NHC(O)—, —NHC(S)—, —OC(NH)—, —OC(O)—, and —OC(S)—, optionally wherein X is —CH₂C(O)— or —NHC(S)—;
Z is —CH₂—, —CH₂C(NH)—, —CH₂C(O)—, —CH₂C(S)—, —NHC(NH)—, —NHC(O)—, —NHC(S)—, —OC(NH)—, —OC(O)—, and —OC(S)—; optionally wherein Z is —CH₂— or —CH₂C(O)—;
L is a peptide linker having 1-6 amino acid residues, inclusive;
R¹ is H or C₁₋₆ alkyl, optionally wherein R¹ is H or methyl;
R² is C₁₋₆ alkyl or C₃₋₆ cycloalkyl; and
R³ is C₁₋₆ alkyl.

In some examples, B is a 6-membered ring. In particular examples, B is a 6-membered aryl. In other examples, B can be a 6-membered heterocyclyl.

In some examples, X can be —CH₂C(O)—. In other examples, X can be —NHC(S)—.

In some examples, Z may be —CH₂— (e.g., when B is a piperidyl ring). Alternatively, Z may be —CH₂C(O)— (e.g., when B is a piperazyl ring).

In some embodiments, R¹ is H and R² is C₄ alkyl, for example, as in compounds of formula (IIa):

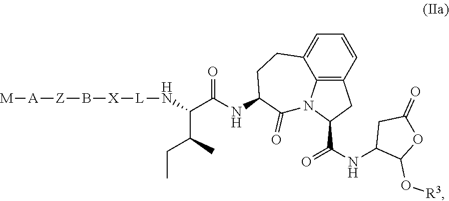

in which variables M, A, B, X, Z, R³, and L are as defined herein.

In further embodiments, X is —CH₂C(O)—, for example, as in compounds of formula (IIb)

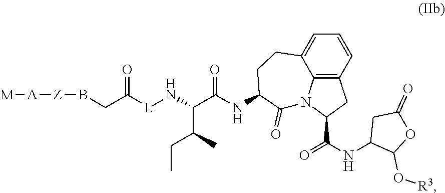

in which variables M, A, B, Z, R³, and L are as defined herein.

In some embodiments, R³ is methyl, as in formula (IIc):

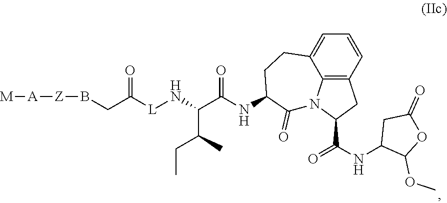

in which variables M, A, B, Z and L are each as defined herein.

In some embodiments, B is piperidyl, and Z is —CH₂— as in formula (IIc-A):

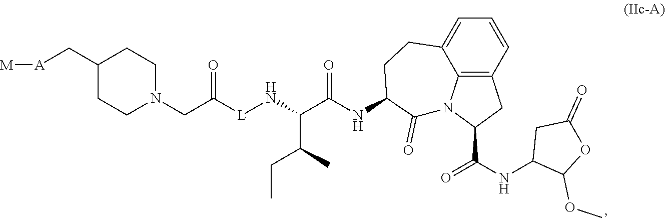

in which variables M, A, and L are each defined herein.

In some embodiments, the compound has defined stereochemistry as in formula (IIc-Aa):

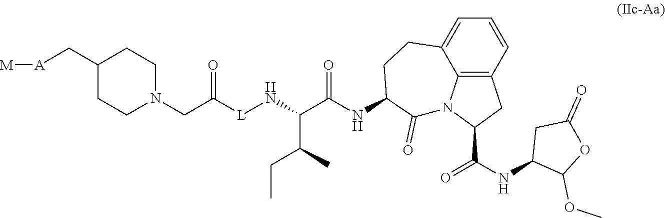

in which variables M, A, and L are each as defined herein.

In particular examples, the compound has defined stereochemistry as in formula (IIc-Ab):

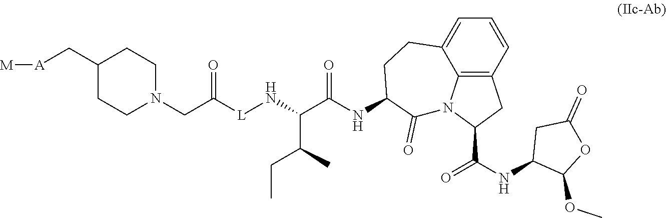

in which variables M, A, and L are each as defined herein.

Exemplary Compounds of formula (IIc-Bb) include Compounds 1-Al to 17-Al as described herein.

In some embodiments, B is phenyl, and Z is —CH₂— as in formula (IIc-B):

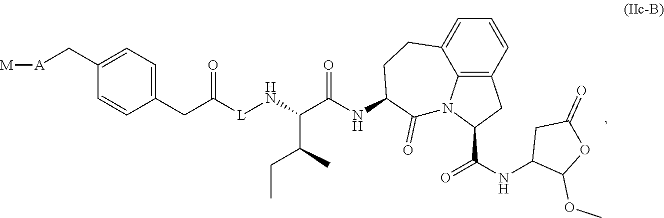

in which variables A and L are each as defined herein.

In some embodiments, the compound has defined stereochemistry as in formula (IIc-Ba):

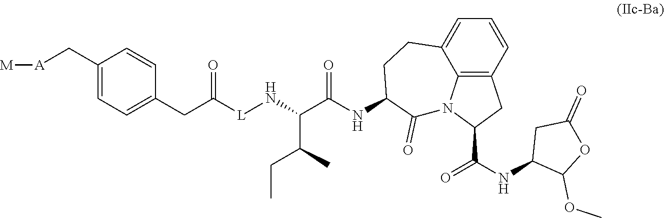

in which variables A and L are each as defined herein.

In particular examples, the compound has defined stereochemistry as in formula (IIc-Bb):

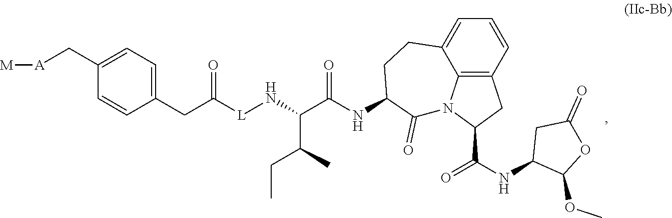

in which variables A and L are each as defined herein.

Exemplary Compounds of formula (IIc-B) include Compounds 18-Al to 28-Al as described herein.

In some embodiments, B is phenyl, and R³ is methyl as in formula (IId):

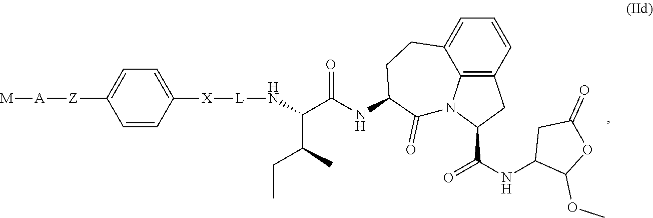

in which variables M, A, Z, X, and L are each as defined herein.

In some embodiments, A is NOTA is —CH₂— as in formula (IId-A):

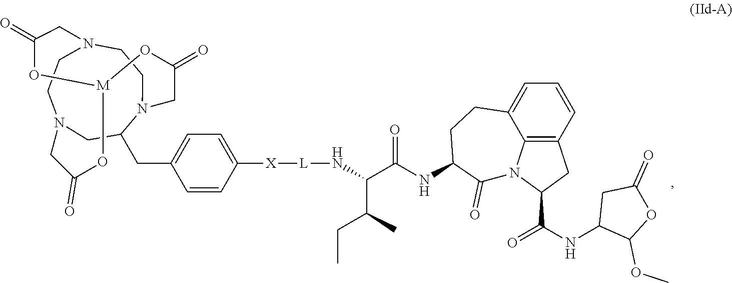

in which variables M, X, and L are each as defined herein.

In some embodiments, the compound has defined stereochemistry as in formula (IId-Aa):

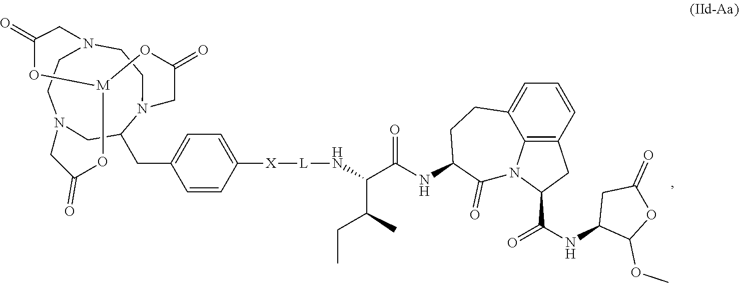

in which variables M, A, X, and L are each as defined herein.

In particular examples, the compound has defined stereochemistry as in formula (IId-Ab):

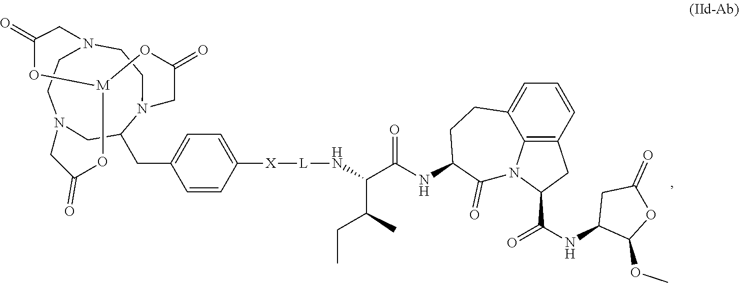

in which variables M, A, X, and L are each as defined herein.

Exemplary Compounds of formula (IId) include Compounds 47-Al to 49-Al as described herein.

In any of the GZB-binding compounds disclosed herein, L may have 1-6 amino acid residues, for example, 1-5 amino acid residues. The amino acid residues may be standard proteinogenic amino acids (the 20 amino acid residues found in naturally-occurring proteins), or unnatural amino acids (e.g., derivatives and/or isomers of any of the 20 naturally-occurring amino acid residues). Structures of exemplary non-naturally occurring amino acid residues that may be included in the L linker are provided in Table 1 below. Exemplary amino acid sequences include Gly, Gly-Gly, Gln-Gly, Glu, Glu-Gly, Glu-Gly-Gly, Glu-βAla-βAla, _{D}Glu, _{D}Glu-βAla-βAla, _{D}Glu-Gly-Gly, _{D}Glu-AEA, _{D}Glu-AEEA-AEEA, _{D}Glu-_{D}Glu-AEA, _{D}Glu-_{D}Glu-βAla-βAla, γGlu, γGlu-βAla, _{D}γGlu, Lys-Gly, Arg-Gly, N-Acid-βAla-βAla, βAla-N-Acid-βAla, βAla-Glu-Gly-Gly, βAla-_{D}Glu-βAla, and Diacid-βAla-βAla. See Table 2 for structures of these exemplary L linkers.

In any of the compounds of formula (I) or (II) described herein, A may be a chelating moiety known in the art to be useful as described herein, e.g., to bind a metal. In some examples, the chelating moiety is 1,4,7-triazacyclononane-N,N′,N″-triacetic acid (NOTA) or 1,4,7-triazacyclononane-4,7-diyl diacetic acid (NODA).

In any of the compounds of formula (II) disclosed herein, M may be a metal known in the art to be useful as described herein, e.g., for imaging purposes. In some examples, the metal (either alone or in conjugation with the radioisope) may be toxic. In other examples, the metal (either alone or in conjunction with the radioisope) may be non-toxic. Exemplary metals include Ga (e.g., ⁶⁸Ga) and Al (which may be conjugated to a radioisotope such as ¹⁸F).

As shown in the exemplary compounds herein, the stereochemistry of the two stereocenters of the 5-oxotetrahydrofuranyl moiety can impact the properties of the compounds disclosed herein. In some examples, the relationship between the amide and the alkoxy is cis, (i.e. syn). In particular examples, the stereocenter of the amide-bearing carbon is assigned (S). In other examples, the stereocenter of the alkoxy-bearing carbon is assigned (R).

In another aspect, the present disclosure features a pharmaceutical composition comprising any of the GZB-binding compounds disclosed herein, a stereoisomer thereof, or a pharmaceutically acceptable salt thereof, and a pharmaceutically acceptable carrier.

In another aspect, the present disclosure features a method of imaging granzyme B in a tissue. The method comprises:
(i) contacting any of the formula (II) compounds disclosed herein, or the pharmaceutically acceptable salt thereof, with a tissue suspected of comprising granzyme B, and
(ii) imaging the tissue based on radioisotope signals released from the compound, or the pharmaceutically acceptable salt thereof.

In some embodiments, the imaging method disclosed herein is performed in vitro. For example, the tissue for imaging by the method is in a biological sample, which may be obtained from a subject (e.g., a human patient) as disclosed herein.

In other embodiments, the imaging method disclosed herein can be performed in vivo, wherein an effective amount of the compound or the pharmaceutically acceptable salt thereof may be administered to a subject (e.g., a human patient) in need thereof.

In some embodiments, the subject is on a treatment (e.g., anti-inflammatory agents, steroids, immunotherapy agents, chemotherapeutic agents, and therapeutic antibodies) for an immunoregulatory abnormality (e.g., an autoimmune disorder, an inflammatory disorder, a skin disorder, a cancer, and a cardiovascular disorder) In some embodiments the immune response in the subject is monitored based on the imaging of granzyme B.

Also provided herein are any of the compounds of formula (II), a pharmaceutically acceptable salt thereof, or a pharmaceutical composition comprising such for use in imaging granzyme B for diagnostic purposes or for monitoring treatment efficacy.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIGS. 1A-1B include diagrams showing synthesis of Compound ¹⁸F-4-Al. FIG. 1A: semiprep HPLC Radio-trace for preparing Compound ¹⁸F-4-Al. FIG. 1B: analytical HPLC Radio-trace for Compound ¹⁸F-4-Al.

FIG. 2 includes diagrams showing structures of exemplary pro-form and active-form of GZB-binding compound. Structures are shown for the active form structure of Compound 29-Al and Compound 4-Al (prodrug form).

FIG. 3 includes a diagram showing the chemical structure of exemplary prodrugs.

FIGS. 4A-4D include diagrams showing in vivo imaging activity of exemplary prodrug Compound ¹⁸F-4-Al (cis-form). FIG. 4A: structure of Compound ¹⁸F-4-Al. FIG. 4B: charts showing in vivo imaging activity of Compound ¹⁸F-4-Al. Left panel: % ID/g. Right panel: TBR. FIG. 4C: a diagram showing presence of the active compound as indicated in mouse plasma 5 minutes after administration. FIG. 4D: a photo showing in vivo imaging activity of Compound ¹⁸F-4-Al.

FIGS. 5A-5D include diagrams showing in vivo imaging activity of exemplary prodrug Compound ¹⁸F-3-Al (trans-form). FIG. 5A: structure of Compound ¹⁸F-3-Al. FIG. 5B: charts showing in vivo imaging activity of Compound ¹⁸F-3-Al. Left panel: % ID/g. Right panel: TBR. FIG. 5C: a diagram showing presence of the compounds as indicated in mouse plasma 5 minutes after administration. FIG. 5D: a photo showing in vivo imaging activity of Compound ¹⁸F-3-Al.

### DETAILED DESCRIPTION

Cancer immunotherapies have represented a significant advance in cancer therapy over recent years. Antibodies directed against immune checkpoints such as programmed cell death protein 1 (PD-1) and cytotoxic T lymphocyte-associated protein 4 (CTLA-4) have been approved with positive outcomes for some patients. Research into the field of immune-oncology continues, with strategies including CAR-T cells, vaccines, small molecules, and antibodies under development. Despite the promise of these therapies, they are not a panacea. These immunotherapies can be associated with significant adverse events, which are costly, and the response rates are typically 20-50%, meaning the majority of patients do not respond to therapy. Furthermore, determining an individual patient's response to therapy can be challenging using conventional methods, as response is frequently associated with an immune-cell infiltrate that can make responding tumors appear to grow on anatomic imaging (e.g., CT, MRI) and demonstrate increased avidity with FDG-PET imaging due to the influx of metabolically active immune cells. Given the constraints of current imaging technologies, clinical studies for cancer immunotherapies typically employ overall survival as their study endpoint as opposed to progression-free survival.

Granzyme B, a downstream marker of cytotoxic T-cell activity, could serve as a novel biomarker to assess cancer immunotherapy efficacy. Granzyme B expression within a tumor can be assessed not only for CTL presence or absence, but also as an effector protein released by active T-cells that also integrates a measure of CTL activity, thus accounting for issues of T-cell exhaustion that make assessment of CTL presence difficult to accomplish.

The present disclosure provides prodrug compounds (a.k.a., pro-form), e.g., Formula (I) compounds and Formula (II) compounds, which are capable of converting to active Granzyme B (GZB)-binding compounds in vivo. Such compounds are chemical stable as compared with the active counterparts. Additionally, the prodrug strategy employed herein allows for reliable stereochemical stability during synthesis of the compounds of Formula (I) and (II), while offering facile liberation of the active drug in vitro. Accordingly, the pro-forms of GZB-binding compounds disclosed herein can serve as Granzyme B imaging agents via rapid in vivo conversion. As reported herein, certain stereochemical isomers (e.g., cis-isomers) showed superior in vivo conversion and imaging activities. The prodrug forms of GZB-binding compounds disclosed herein can be used to identify patients who are responsive to an immunotherapeutic agent or monitoring treatment efficacy of an immunotherapeutic agent based on the level and/or location of Granzyme B as determined by the imaging assay disclosed herein.

### Definitions

It is to be understood that the terminology employed herein is for the purpose of describing particular embodiments and is not intended to be limiting. Further, although any methods, devices, and materials similar or equivalent to those described herein can be used in the practice or testing of the invention, the preferred methods, devices, and materials are now described. In addition to the foregoing, as used in the specification and appended claims, unless specified to the contrary, the following terms have the meaning indicated:

“Amino” refers to the —NH₂ radical.

“Cyano” refers to the —CN radical.

“Hydroxyl” refers to the —OH radical.

“Imino” refers to the ═NH substituent.

“Nitro” refers to the —NO₂ radical.

“Oxo” refers to the ═O substituent.

“Thioxo” refers to the =S substituent.

“Trifluoromethyl” refers to the —CF₃ radical.

“Alkyl” refers to a linear, saturated, acyclic, monovalent hydrocarbon radical or branched, saturated, acyclic, monovalent hydrocarbon radical, having from one to six carbon atoms, and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylpentyl-1,2-methylpentyl and the like. An alkyl moiety may be unsubstituted. Alternatively, an alkyl moiety may be optionally substituted. An optionally substituted alkyl radical is an alkyl radical that is optionally substituted, valence permitting, by one, two, three, four, or five substituents independently selected from the group consisting of halo, cyano, nitro, aryl, cycloalkyl, heterocyclyl, heteroaryl, oxo, trimethylsilanyl, —OR³, —OC(O)—R³, —N(R³)₂, —C(O)R⁴, —C(O)OR³, —C(O)N(R³)₂, —N(R³)C(O)OR⁵, —N(R³)C(O)R⁵, —N(R³)S(O)ₜR⁵ (where t is 1 or 2), —S(O)ₜOR⁵ (where t is 1 or 2), —S(O)ₚR⁵ (where p is 0, 1, or 2) and —S(O)ₜN(R³)₂ (where t is 1 or 2), where each R³ is independently hydrogen, alkyl, haloalkyl, cycloalkyl, cycloalkylalkyl, aryl, aralkyl, heterocyclyl, or heteroaryl; each R⁴ is independently hydrogen, cycloalkyl, aryl, heterocyclyl, or heteroaryl; and each R⁵ is independently alkyl, haloalkyl, cycloalkyl, aryl, heterocyclyl, or heteroaryl.

“Cycloalkyl” refers to a stable non-aromatic monocyclic or polycyclic hydrocarbon radical having from three to fifteen carbon atoms, preferably having from three to ten carbon atoms, and which is saturated or unsaturated, and which attaches to the rest of the molecule by a single bond. A polycyclic hydrocarbon radical is bicyclic, tricyclic, or tetracyclic ring system. An unsaturated cycloalkyl contains one, two, or three carbon-carbon double bonds and/or one carbon-carbon triple bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, and the like. A cycloalkyl moiety may be unsubstituted. Alternatively, a cycloalkyl moiety may be optionally substituted. An optionally substituted cycloalkyl is a cycloalkyl radical that is optionally substituted by one, two, three, four, or five substituents independently selected from the group consisting of alkyl, alkenyl, halo, haloalkyl, haloalkenyl, cyano, nitro, oxo, aryl, aralkyl, cycloalkyl, heterocyclyl, heteroaryl, —R⁴—OR³, —R⁴—OC(O)—R³, —R⁴—N(R³)₂, —R⁴—C(O)R³, R⁴—C(O)OR³, —R⁴—C(O)N(R³)₂, —R⁴—N(R³)C(O)OR⁵, —R⁴—N(R³)C(O)R⁵, —R⁴—N(R³)S(O)ₜR⁵ (where t is 1 or 2), —R⁴—S(O)ₜOR⁵ (where t is 1 or 2), —R⁴—S(O)ₚR⁵ (where p is 0, 1, or 2) and —R⁴—S(O)ₜN(R³)₂ (where t is 1 or 2) where each R³ is independently hydrogen, alkyl, haloalkyl, cycloalkyl, aryl, heterocyclyl, or heteroaryl; each R⁴ is independently a direct bond or a linear or branched alkylene or alkenylene chain; and each R⁵ is independently alkyl, haloalkyl, cycloalkyl, cycloalkylalkyl, aryl, aralkyl, heterocyclyl, or heteroaryl.

“Chelating moieties” are those molecules or ions, which are able to act as a polydentate ligand to a metal ion. For example, molecules with multiple atoms with available lone pairs (including but not limited to nitrogen and oxygen) may act as chelating moieties. Chelating moieties may be linear (e.g., EDTA), or cyclic (including macrocycles e.g., DOTA, porphyrin) and may involve macrocyas commonly known in the art. Chelating moieties may have 2, 3, 4, 5, or 6 functional groups (e.g., amines, amides, hydroxyls, carboxylic acids etc.) with available lone pairs to coordinate with a metal.

In some embodiments, preparation of compounds can involve the addition of acids or bases to affect, for example, catalysis of a desired reaction or formation of salt forms such as acid addition salts.

Exemplary acids can be inorganic or organic acids and include, but are not limited to, strong and weak acids. Some example acids include hydrochloric acid, hydrobromic acid, sulfuric acid, phosphoric acid, p-toluenesulfonic acid, 4-nitrobenzoic acid, methanesulfonic acid, benzenesulfonic acid, trifluoroacetic acid, and nitric acid. Some weak acids include, but are not limited to acetic acid, propionic acid, butanoic acid, benzoic acid, tartaric acid, pentanoic acid, hexanoic acid, heptanoic acid, octanoic acid, nonanoic acid, and decanoic acid.

Exemplary bases include lithium hydroxide, sodium hydroxide, potassium hydroxide, lithium carbonate, sodium carbonate, potassium carbonate, and sodium bicarbonate. Some example strong bases include, but are not limited to, hydroxide, alkoxides, metal amides, metal hydrides, metal dialkylamides and arylamines, wherein; alkoxides include lithium, sodium and potassium salts of methyl, ethyl and t-butyl oxides; metal amides include sodium amide, potassium amide and lithium amide; metal hydrides include sodium hydride, potassium hydride and lithium hydride; and metal dialkylamides include lithium, sodium, and potassium salts of methyl, ethyl, n-propyl, iso-propyl, n-butyl, tert-butyl, trimethylsilyl and cyclohexyl substituted amides.

As used herein, the phrase “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present application include the conventional non-toxic salts of the parent compound formed, for example, from non-toxic inorganic or organic acids. The pharmaceutically acceptable salts of the present application can be synthesized from the parent compound, which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (MeCN) are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977). Conventional methods for preparing salt forms are described, for example, in Handbook of Pharmaceutical Salts: Properties, Selection, and Use, Wiley-VCH, 2002.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof. Methods for isolating compounds and their salts are routine in the art.

The expressions “ambient temperature” and “room temperature” or “rt” as used herein, are understood in the art, and refer generally to a temperature, e.g. a reaction temperature, that is about the temperature of the room in which the reaction is carried out, for example, a temperature from about 20° C. to about 30° C.

### I. Prodrugs of Granzyme B-Targeting Compounds

In some aspects, provided herein are prodrugs of Granzyme B-targeting compounds disclosed herein, e.g., Formula (I) or Formula (II) compounds. The compounds disclosed herein encompass the compounds per se, their pharmaceutically acceptable salt thereof, and stereoisomers thereof.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-performance liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers.

### A. Compounds of Formula (I)

In some embodiments, the disclosure provides compounds of Formula (I), shown below, which are prodrugs of compounds capable of binding to Granzyme B with high binding affinity and/or specificity:

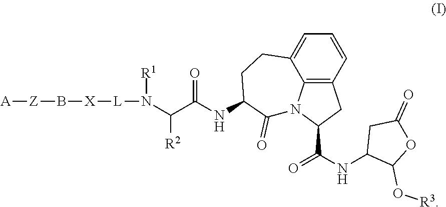

In Formula (I), A is a chelating moiety. Exemplary chelating moieties for use in the Granzyme B-targeting compounds disclosed herein include, but are not limited to, 1,4,7-triazacyclononanetriacetic acid (NOTA), 1,4,7,10-tetraazacyclododecane-1,4,7,10-tetraacetic acid (DOTA), 1,4,7-triazacyclononane-1-glutaric acid-4,7-diacetic acid (NODAGA), ethylene diamine tetra-acetic acid (EDTA), diethylene triaminepentaacetic acid (DTPA), cyclohexyl-1,2-diaminetetraacetic acid (CDTA), ethyleneglycol-0,0′-bis(2-aminoethyl)-N,N,N′,N′-tetraacetic acid (EGTA), N,N-bis(hydroxybenzyl)-ethylenediamine-N,N′-diacetic acid (HBED), triethylene tetramine hexaacetic acid (TTHA), hydroxyethyidiamine triacetic acid (HEDTA), 1,4,8,11-tetraazacyclotetradecane-N,N′,N″,N′″-tetraacetic acid (TETA), 1,4,7,10-tetraaza-1,4,7,10-tetra-(2-carbamoyl methyl)-cyclododecane (TCMC), 1,4,7-triazacyclononane-4,7-diyl diacetic acid (NODA) and Desferrioxamine B (DFO). In some embodiments, the chelating agent is selected from the group consisting of 1,4,7-triazacyclononanetriacetic acid (NOTA), 1,4,7,10-tetraazacyclododecane-1,4,7,10-tetraacetic acid (DOTA), 1,4,7-triazacyclononane-4,7-diyl diacetic acid (NODA) and 1,4,7-triazacyclononane-1-glutaric acid-4,7-diacetic acid (NODAGA). In some embodiments, the chelating agent is 1,4,7-triazacyclononanetriacetic acid (NOTA). In other embodiments, the chelating agent is 1,4,7,10-tetraazacyclododecane-1,4,7,10-tetraacetic acid (DOTA). In some embodiments, the chelating agent is 1,4,7-triazacyclononane-4,7-diyl diacetic acid (NODA).

B can be aryl, heteroaryl, cycloalkyl, or heterocyclyl. In some examples, B is a 6-membered ring. In one example B is a 6-membered aryl ring, for example, phenyl. In another, B is a 6-membered heterocyclyl, for example, a piperdine ring or a piperazine ring.

X can be —CH₂C(NH)—, —CH₂C(O)—, —CH₂C(S)—, —NHC(NH)—, —NHC(O)—, —NHC(S)—, —OC(NH)—, —OC(O)—, or —OC(S)—. In one example, X is —CH₂C(O)—. In another example, X is —NHC(S)—.

Z can be —CH₂—, —CH₂C(NH)—, —CH₂C(O)—, —CH₂C(S)—, —NHC(NH)—, —NHC(O)—, —NHC(S)—, —OC(NH)—, —OC(O)—, and —OC(S)—. In some embodiments, Z is —CH₂— (e.g., when in connection with a piperdine ring). In other embodiments, Z is —CH₂C(O)— (e.g., when in connection with a piperazine ring).

L can be a peptide linker having 1-6 amino acid residues, inclusive. In some examples, L includes 1-5 amino acid residues, inclusive. In other examples, L includes 2-4 amino acid residues, inclusive. In one example, L includes 1 amino acid residue. In another example, L includes 2 amino acid. In yet another example, L includes 3 amino acid residues. Alternatively, L includes 4 amino acid residues. In still another example, L includes 5 amino acid residues. Alternatively, L includes 6 amino acid residues.

Compatible amino acid residues in the peptide linker L may include natural and non-natural amino acid residues (including β-amino acid residues and D-amino acids) and is not limited to proteinogenic amino acid residues. As used herein, proteinogenic amino acid residues refer to the 20 amino acid residues existing in nature as building blocks for synthesizing proteins. Amino acid residues may form a chain through standard peptide bonds, or by forming amide bonds with compatable side chains (e.g., glutamic acid (e.g., D-Glu), aspartic acid). Table 1 provides some exemplary non-proteinogenic (non-naturally occurring) amino acids that can be used in any of the peptide linker L disclosed herein, including their chemical structures.

TABLE 1
Exemplary Non-Proteinogenic Amino Acids

| Chemical Structure | Non-Naturally Occurring Amino Acids |
|---|---|
| 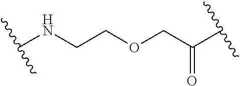 [SMILES: *NCCOCC(*)=O] | AEA |
| 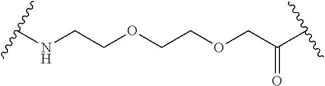 [SMILES: *NCCOCCOCC(*)=O] | AEEA |
| 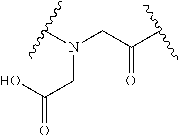 [SMILES: *C(=O)CN(*)CC(=O)O] | N-acid |
| 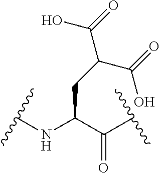 [SMILES: *N[C@@H](CC(C(=O)O)C(=O)O)C(*)=O] | Diacid |
| 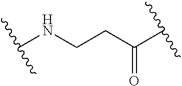 [SMILES: *NCCC(*)=O] | βAla |
| 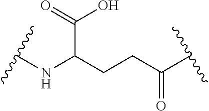 [SMILES: *NC(CCC(*)=O)C(=O)O] | γGlu |

Exemplary peptide linkers include, but are not limited to, Gly, Gly-Gly, Gln-Gly, Glu, Glu-Gly, Glu-Gly-Gly, Glu-βAla-βAla, _{D}Glu, _{D}Glu-βAla-βAla, _{D}Glu-Gly-Gly, _{D}Glu-AEA, _{D}Glu-AEEA-AEEA, _{D}Glu-_{D}Glu-AEA, _{D}Glu-_{D}Glu-βAla-βAla, γGlu, γGlu-βAla, _{D}γGlu, Lys-Gly, Arg-Gly, N-Acid-βAla-βAla, βAla-N-Acid-βAla, βAla-Glu-Gly-Gly, βAla-_{D}Glu-βAla, and Diacid-βAla-βAla.

TABLE 2
Exemplary Peptide Linkers

| | |
|---|---|
| 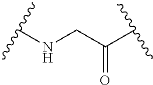 [SMILES: *NCC(*)=O] | Gly |
| 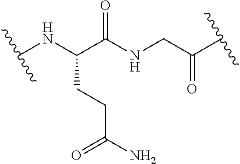 [SMILES: *N[C@@H](CCC(N)=O)C(=O)NCC(*)=O] | Gln-Gly |
| 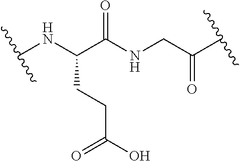 [SMILES: *N[C@@H](CCC(=O)O)C(=O)NCC(*)=O] | Glu-Gly |
| 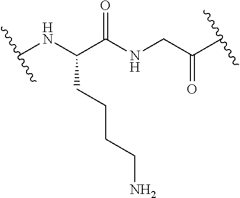 [SMILES: *N[C@@H](CCCCN)C(=O)NCC(*)=O] | Lys-Gly |
| 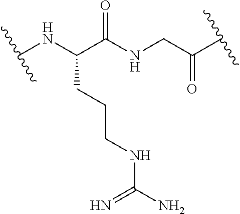 [SMILES: *N[C@@H](CCCNC(=N)N)C(=O)NCC(*)=O] | Arg-Gly |
| 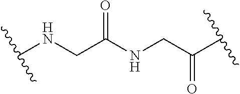 [SMILES: *NCC(=O)NCC(*)=O] | Gly-Gly |
| 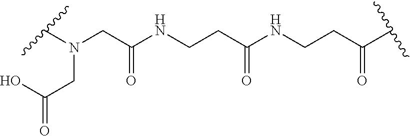 [SMILES: *C(=O)CCNC(=O)CCNC(=O)CN(*)CC(=O)O] | N-Acid-βAla-βAla |
| 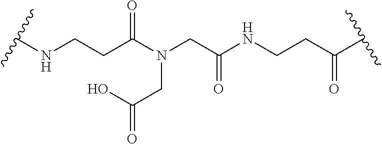 [SMILES: *NCCC(=O)N(CC(=O)O)CC(=O)NCCC(*)=O] | βAla-N-Acid-βAla |
| 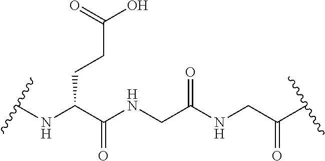 [SMILES: *N[C@H](CCC(=O)O)C(=O)NCC(=O)NCC(*)=O] | _{D}Glu-Gly-Gly |
| 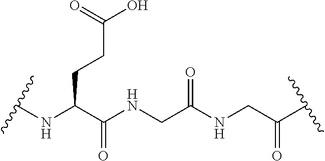 [SMILES: *N[C@@H](CCC(=O)O)C(=O)NCC(=O)NCC(*)=O] | Glu-Gly-Gly |
| 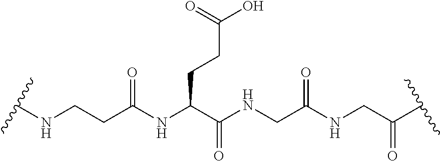 [SMILES: *NCCC(=O)N[C@@H](CCC(=O)O)C(=O)NCC(=O)NCC(*)=O] | βAla-Glu-Gly-Gly |
| 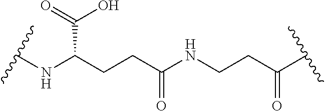 [SMILES: *N[C@@H](CCC(=O)NCCC(*)=O)C(=O)O] | γGlu-βAla |
| 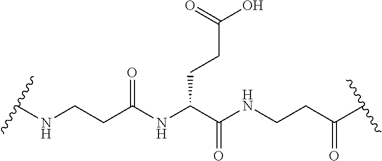 [SMILES: *NCCC(=O)N[C@H](CCC(=O)O)C(=O)NCCC(*)=O] | βAla-_{D}Glu-βAla |
| 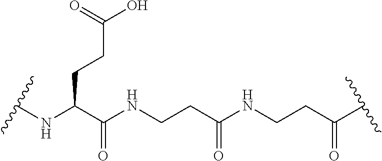 [SMILES: *N[C@@H](CCC(=O)O)C(=O)NCCC(=O)NCCC(*)=O] | Glu-βAla-βAla |
| 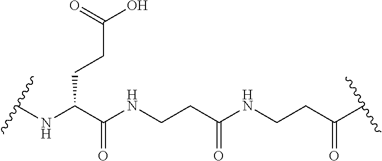 [SMILES: *N[C@H](CCC(=O)O)C(=O)NCCC(=O)NCCC(*)=O] | _{D}Glu-βAla-βAla |
| 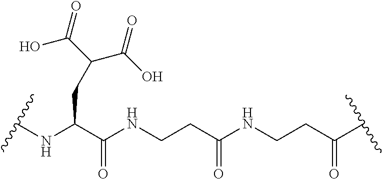 [SMILES: *N[C@@H](CC(C(=O)O)C(=O)O)C(=O)NCCC(=O)NCCC(*)=O] | Diacid-βAla-βAla |
| 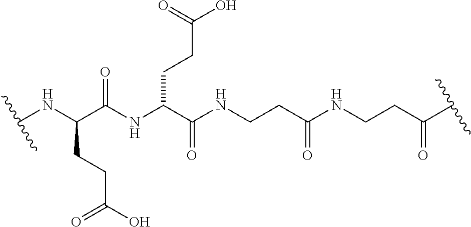 [SMILES: *N[C@H](CCC(=O)O)C(=O)N[C@H](CCC(=O)O)C(=O)NCCC(=O)NCCC(*)=O] | _{D}Glu-_{D}Glu-βAla-βAla |
| 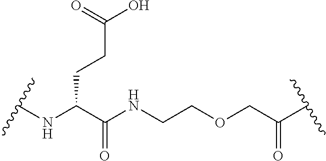 [SMILES: *N[C@H](CCC(=O)O)C(=O)NCCOCC(*)=O] | _{D}Glu-AEA |
| 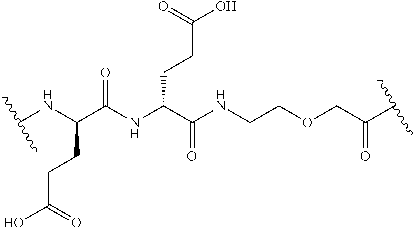 [SMILES: *N[C@H](CCC(=O)O)C(=O)N[C@H](CCC(=O)O)C(=O)NCCOCC(*)=O] | _{D}Glu-_{D}Glu-AEA |
| 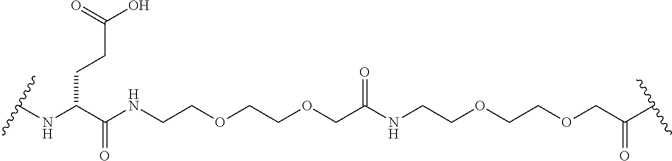 [SMILES: *N[C@H](CCC(=O)O)C(=O)NCCOCCOCC(=O)NCCOCCOCC(*)=O] | _{D}Glu-AEEA-AEEA |
| 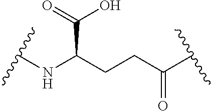 [SMILES: *N[C@H](CCC(*)=O)C(=O)O] | _{D}γGlu |
| 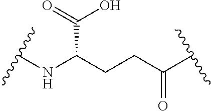 [SMILES: *N[C@@H](CCC(*)=O)C(=O)O] | γGlu |
| 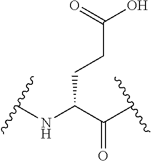 [SMILES: *N[C@H](CCC(=O)O)C(*)=O] | _{D}Glu |
| 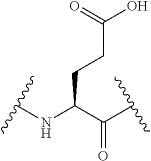 [SMILES: *N[C@@H](CCC(=O)O)C(*)=O] | Glu |

In some embodiments, R¹ is H. In other embodiments, R¹ is C₁₋₆ alkyl. For example, R¹ can be methyl.

In some embodiments, R² can be C₁₋₆ alkyl. Alternatively, R² can be C₃₋₆ alkyl (e.g., branched or unbranched, substituted or unsubstituted) or C₃₋₆ cycloalkyl (e.g., cyclopropyl, cyclobutyl, cyclopentyl, cyclohexyl).

In some embodiments, R¹ is H, and R² is C₄ alkyl as in compounds of Formula (Ia):

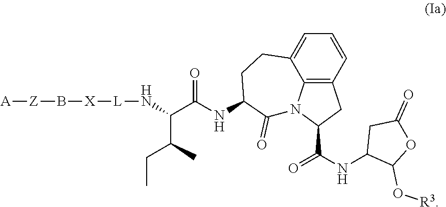

In some specific examples, X is —CH₂C(O)—, as in compounds of Formula (Ib):

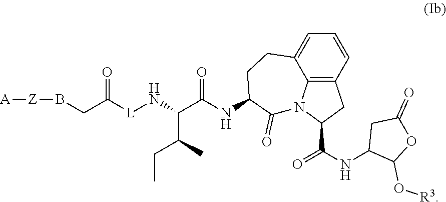

In some examples, R³ is methyl, as in compounds of Formula (Ic):

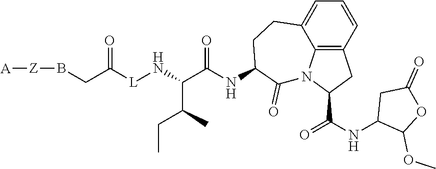

In some examples of Formula (Ic), B is piperidyl, and Z is —CH₂— as in compounds of Formula (Ic-A):

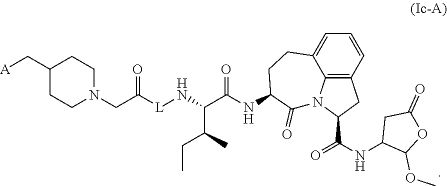

Exemplary compounds of Formula (Ic-A) include those listed in Table 3.

TABLE 3
Exemplary Compounds of Formula (Ic-A)

| Compounds | ID |
|---|---|
| 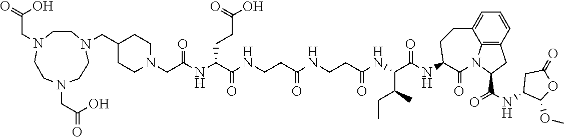 [SMILES: CC[C@H](C)[C@H](NC(=O)CCNC(=O)CCNC(=O)[C@@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)N[C@@H]1CC(=O)O[C@@H]1OC)C3] | 1 |
| 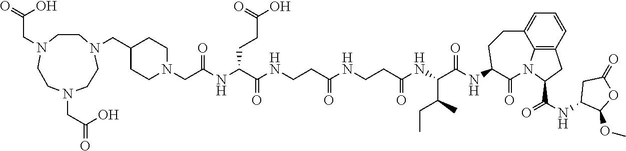 [SMILES: CC[C@H](C)[C@H](NC(=O)CCNC(=O)CCNC(=O)[C@@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)N[C@@H]1CC(=O)O[C@H]1OC)C3] | 2 |
| 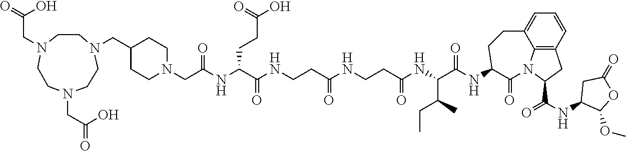 [SMILES: CC[C@H](C)[C@H](NC(=O)CCNC(=O)CCNC(=O)[C@@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)N[C@H]1CC(=O)O[C@@H]1OC)C3] | 3 |
| 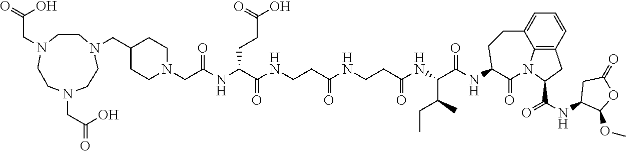 [SMILES: CC[C@H](C)[C@H](NC(=O)CCNC(=O)CCNC(=O)[C@@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)N[C@H]1CC(=O)O[C@H]1OC)C3] | 4 |
| 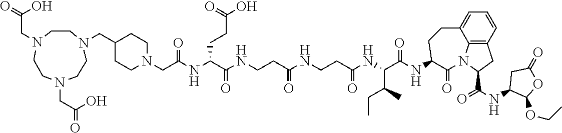 [SMILES: CCO[C@@H]1OC(=O)C[C@@H]1NC(=O)[C@@H]1Cc2cccc3c2N1C(=O)[C@@H](NC(=O)[C@@H](NC(=O)CCNC(=O)CCNC(=O)[C@@H](CCC(=O)O)NC(=O)CN1CCC(CN2CCN(CC(=O)O)CCN(CC(=O)O)CC2)CC1)[C@@H](C)CC)CC3] | 5 |
| 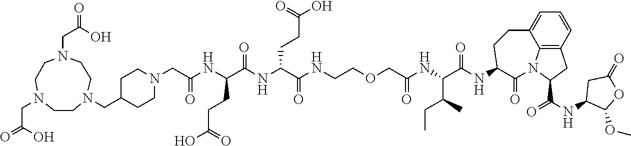 [SMILES: CC[C@H](C)[C@H](NC(=O)COCCNC(=O)[C@@H](CCC(=O)O)NC(=O)[C@@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)N[C@H]1CC(=O)O[C@@H]1OC)C3] | 6 |
| 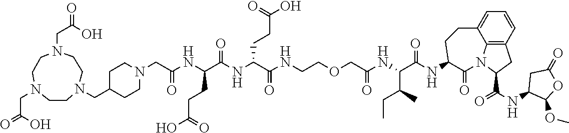 [SMILES: CC[C@H](C)[C@H](NC(=O)COCCNC(=O)[C@@H](CCC(=O)O)NC(=O)[C@@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)N[C@H]1CC(=O)O[C@H]1OC)C3] | 7 |
| 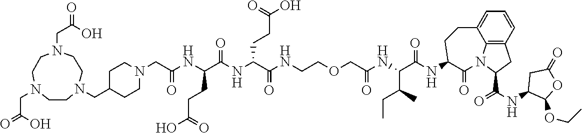 [SMILES: CCO[C@@H]1OC(=O)C[C@@H]1NC(=O)[C@@H]1Cc2cccc3c2N1C(=O)[C@@H](NC(=O)[C@@H](NC(=O)COCCNC(=O)[C@@H](CCC(=O)O)NC(=O)[C@@H](CCC(=O)O)NC(=O)CN1CCC(CN2CCN(CC(=O)O)CCN(CC(=O)O)CC2)CC1)[C@@H](C)CC)CC3] | 8 |
| 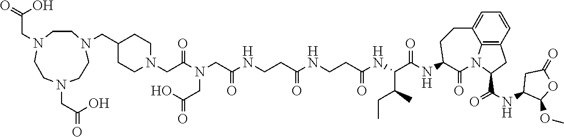 [SMILES: CC[C@H](C)[C@H](NC(=O)CCNC(=O)CCNC(=O)CN(CC(=O)O)C(=O)CN1CCC(CN2CCN(CC(=O)O)CCN(CC(=O)O)CC2)CC1)C(=O)N[C@H]1CCc2cccc3c2N(C1=O)[C@H](C(=O)N[C@H]1CC(=O)O[C@H]1OC)C3] | 9 |
| 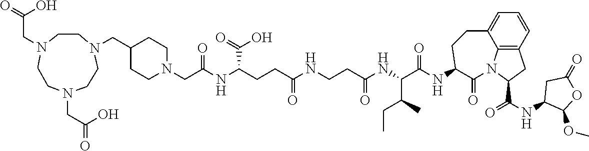 [SMILES: CC[C@H](C)[C@H](NC(=O)CCNC(=O)CC[C@H](NC(=O)CN1CCC(CN2CCN(CC(=O)O)CCN(CC(=O)O)CC2)CC1)C(=O)O)C(=O)N[C@H]1CCc2cccc3c2N(C1=O)[C@H](C(=O)N[C@H]1CC(=O)O[C@H]1OC)C3] | 10 |
| 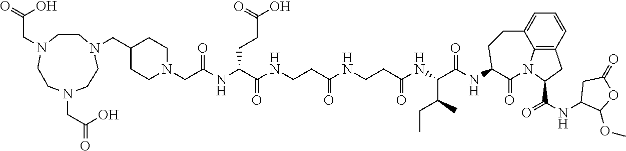 [SMILES: CC[C@H](C)[C@H](NC(=O)CCNC(=O)CCNC(=O)[C@@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)NC1CC(=O)OC1OC)C3] | 11 |
| 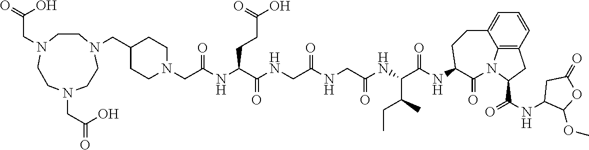 [SMILES: CC[C@H](C)[C@H](NC(=O)CNC(=O)CNC(=O)[C@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)NC1CC(=O)OC1OC)C3] | 12 |
| 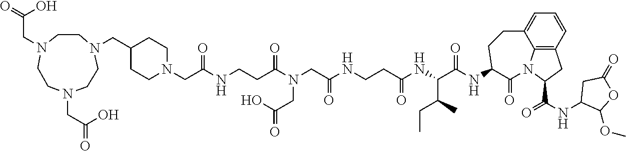 [SMILES: CC[C@H](C)[C@H](NC(=O)CCNC(=O)CN(CC(=O)O)C(=O)CCNC(=O)CN1CCC(CN2CCN(CC(=O)O)CCN(CC(=O)O)CC2)CC1)C(=O)N[C@H]1CCc2cccc3c2N(C1=O)[C@H](C(=O)NC1CC(=O)OC1OC)C3] | 13 |
| 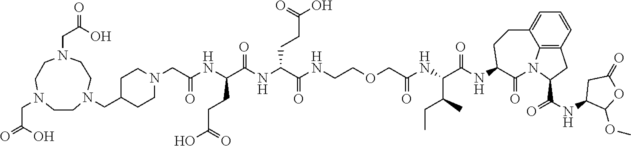 [SMILES: CC[C@H](C)[C@H](NC(=O)COCCNC(=O)[C@@H](CCC(=O)O)NC(=O)[C@@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)N[C@H]1CC(=O)OC1OC)C3] | 14 |
| 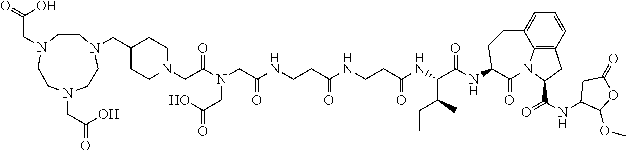 [SMILES: CC[C@H](C)[C@H](NC(=O)CCNC(=O)CCNC(=O)CN(CC(=O)O)C(=O)CN1CCC(CN2CCN(CC(=O)O)CCN(CC(=O)O)CC2)CC1)C(=O)N[C@H]1CCc2cccc3c2N(C1=O)[C@H](C(=O)NC1CC(=O)OC1OC)C3] | 15 |
| 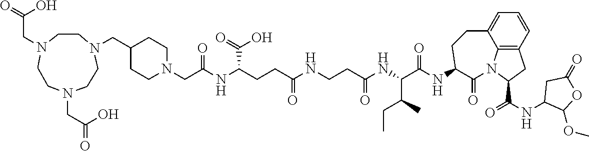 [SMILES: CC[C@H](C)[C@H](NC(=O)CCNC(=O)CC[C@H](NC(=O)CN1CCC(CN2CCN(CC(=O)O)CCN(CC(=O)O)CC2)CC1)C(=O)O)C(=O)N[C@H]1CCc2cccc3c2N(C1=O)[C@H](C(=O)NC1CC(=O)OC1OC)C3] | 16 |
| 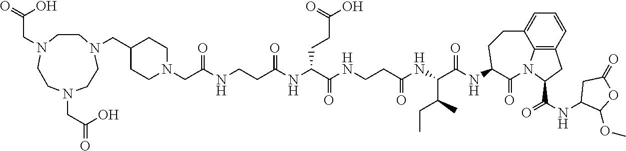 [SMILES: CC[C@H](C)[C@H](NC(=O)CCNC(=O)[C@@H](CCC(=O)O)NC(=O)CCNC(=O)CN1CCC(CN2CCN(CC(=O)O)CCN(CC(=O)O)CC2)CC1)C(=O)N[C@H]1CCc2cccc3c2N(C1=O)[C@H](C(=O)NC1CC(=O)OC1OC)C3] | 17 |

In some examples, the compound of Formula (I) is Compound 1, 2, 3, 4, 5, 6, 7, 8, 9, 10, 11, 12, 13, 14, 15, 16, or 17. In one specific example, the compound of Formula (I) is Compound 4. In another specific example, the compound of Formula (I) is Compound 7. As described herein, the above examples benefit from the piperidine ring, which exhibits improved properties over other related moieties.

In some examples of Formula (Ic), B is piperazyl, and Z is —CH₂C(O)— as in compounds of Formula (Ic-B):

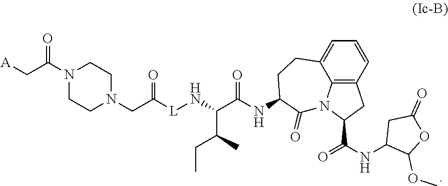

Exemplary compounds of Formula (Ic-B) include those listed in Table 4.

TABLE 4
Exemplary Compounds of Formula (Ic-B)

| Compounds | ID |
|---|---|
| 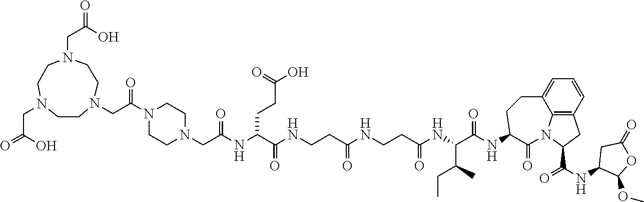 [SMILES: CC[C@H](C)[C@H](NC(=O)CCNC(=O)CCNC(=O)[C@@H](CCC(=O)O)NC(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)N[C@H]1CC(=O)O[C@H]1OC)C3] | 18 |
| 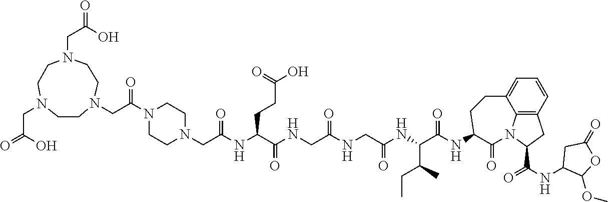 [SMILES: CC[C@H](C)[C@H](NC(=O)CNC(=O)CNC(=O)[C@H](CCC(=O)O)NC(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)NC1CC(=O)OC1OC)C3] | 19 |
| 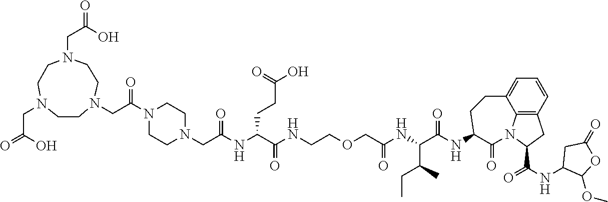 [SMILES: CC[C@H](C)[C@H](NC(=O)COCCNC(=O)[C@@H](CCC(=O)O)NC(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)NC1CC(=O)OC1OC)C3] | 20 |
| 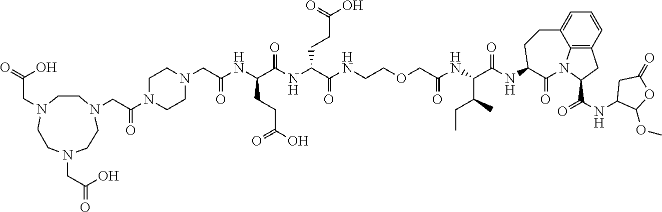 [SMILES: CC[C@H](C)[C@H](NC(=O)COCCNC(=O)[C@@H](CCC(=O)O)NC(=O)[C@@H](CCC(=O)O)NC(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)NC1CC(=O)OC1OC)C3] | 21 |
| 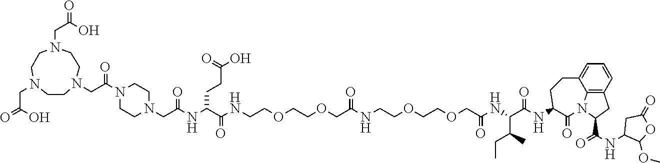 [SMILES: CC[C@H](C)[C@H](NC(=O)COCCOCCNC(=O)COCCOCCNC(=O)[C@@H](CCC(=O)O)NC(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)NC1CC(=O)OC1OC)C3] | 22 |
| 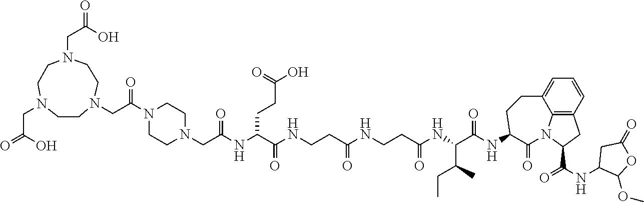 [SMILES: CC[C@H](C)[C@H](NC(=O)CCNC(=O)CCNC(=O)[C@@H](CCC(=O)O)NC(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)NC1CC(=O)OC1OC)C3] | 23 |
| 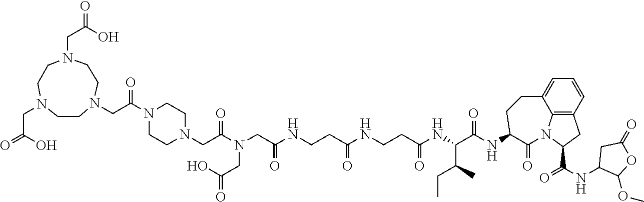 [SMILES: CC[C@H](C)[C@H](NC(=O)CCNC(=O)CCNC(=O)CN(CC(=O)O)C(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)NC1CC(=O)OC1OC)C3] | 24 |
| 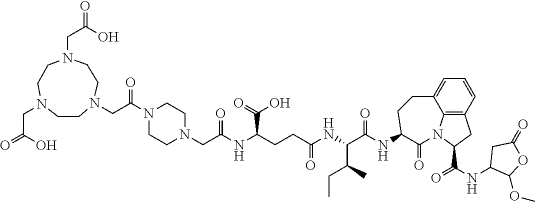 [SMILES: CC[C@H](C)[C@H](NC(=O)CC[C@@H](NC(=O)CN1CCN(C(=O)CN2CCN(CC(=O)O)CCN(CC(=O)O)CC2)CC1)C(=O)O)C(=O)N[C@H]1CCc2cccc3c2N(C1=O)[C@H](C(=O)NC1CC(=O)OC1OC)C3] | 25 |
| 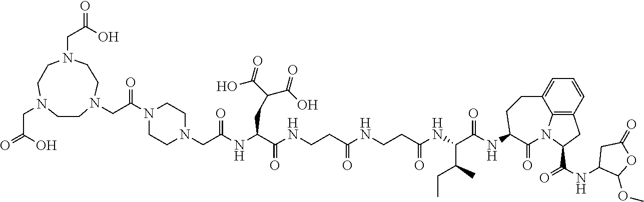 [SMILES: CC[C@H](C)[C@H](NC(=O)CCNC(=O)CCNC(=O)[C@H](CC(C(=O)O)C(=O)O)NC(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)NC1CC(=O)OC1OC)C3] | 26 |
| 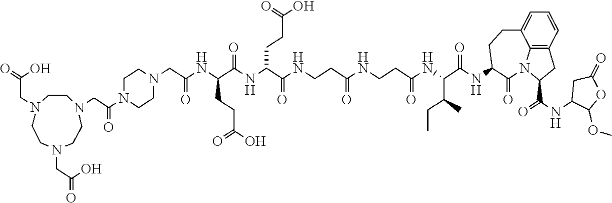 [SMILES: CC[C@H](C)[C@H](NC(=O)CCNC(=O)CCNC(=O)[C@@H](CCC(=O)O)NC(=O)[C@@H](CCC(=O)O)NC(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)NC1CC(=O)OC1OC)C3] | 27 |
| 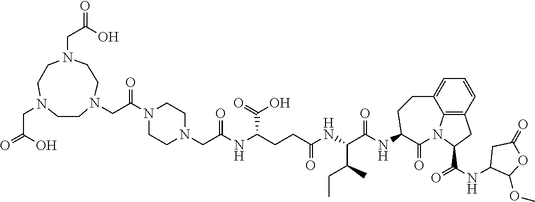 [SMILES: CC[C@H](C)[C@H](NC(=O)CC[C@H](NC(=O)CN1CCN(C(=O)CN2CCN(CC(=O)O)CCN(CC(=O)O)CC2)CC1)C(=O)O)C(=O)N[C@H]1CCc2cccc3c2N(C1=O)[C@H](C(=O)NC1CC(=O)OC1OC)C3] | 28 |

In some examples, the compound of Formula (I) is Compound 18, 19, 20, 21, 22, 23, 24, 25, 26, 27, or 28. As described herein, the above examples benefit from the piperazine ring, which exhibits improved properties over some other related moieties.

In some examples of Formula (Ic), B is phenyl, and Z is —CH₂— as in compounds of Formula (Ic-C):

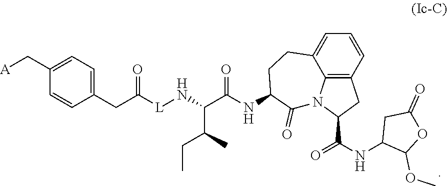

Exemplary compounds of Formula (Ic-C) include those listed in Table 5.

TABLE 5
Exemplary Compounds of Formula (Ic-C)

| Compounds | ID |
|---|---|
| 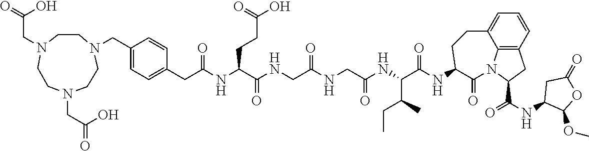 [SMILES: CC[C@H](C)[C@H](NC(=O)CNC(=O)CNC(=O)[C@H](CCC(=O)O)NC(=O)Cc1ccc(CN2CCN(CC(=O)O)CCN(CC(=O)O)CC2)cc1)C(=O)N[C@H]1CCc2cccc3c2N(C1=O)[C@H](C(=O)N[C@H]1CC(=O)O[C@H]1OC)C3] | 29 |
| 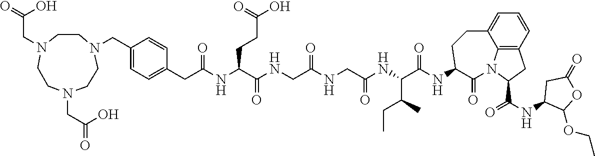 [SMILES: CCOC1OC(=O)C[C@@H]1NC(=O)[C@@H]1Cc2cccc3c2N1C(=O)[C@@H](NC(=O)[C@@H](NC(=O)CNC(=O)CNC(=O)[C@H](CCC(=O)O)NC(=O)Cc1ccc(CN2CCN(CC(=O)O)CCN(CC(=O)O)CC2)cc1)[C@@H](C)CC)CC3] | 30 |
| 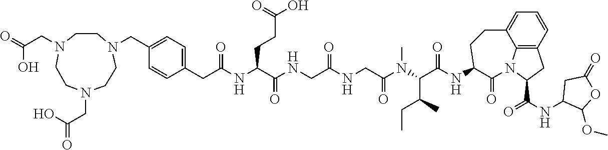 [SMILES: CC[C@H](C)[C@@H](C(=O)N[C@H]1CCc2cccc3c2N(C1=O)[C@H](C(=O)NC1CC(=O)OC1OC)C3)N(C)C(=O)CNC(=O)CNC(=O)[C@H](CCC(=O)O)NC(=O)Cc1ccc(CN2CCN(CC(=O)O)CCN(CC(=O)O)CC2)cc1] | 31 |
| 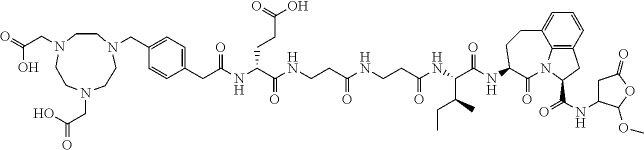 [SMILES: CC[C@H](C)[C@H](NC(=O)CCNC(=O)CCNC(=O)[C@@H](CCC(=O)O)NC(=O)Cc1ccc(CN2CCN(CC(=O)O)CCN(CC(=O)O)CC2)cc1)C(=O)N[C@H]1CCc2cccc3c2N(C1=O)[C@H](C(=O)NC1CC(=O)OC1OC)C3] | 32 |
| 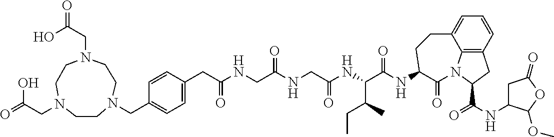 [SMILES: CC[C@H](C)[C@H](NC(=O)CNC(=O)CNC(=O)Cc1ccc(CN2CCN(CC(=O)O)CCN(CC(=O)O)CC2)cc1)C(=O)N[C@H]1CCc2cccc3c2N(C1=O)[C@H](C(=O)NC1CC(=O)OC1OC)C3] | 33 |
| 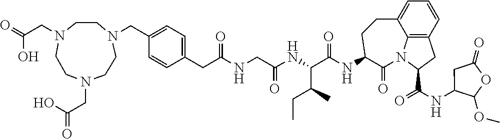 [SMILES: CC[C@H](C)[C@H](NC(=O)CNC(=O)Cc1ccc(CN2CCN(CC(=O)O)CCN(CC(=O)O)CC2)cc1)C(=O)N[C@H]1CCc2cccc3c2N(C1=O)[C@H](C(=O)NC1CC(=O)OC1OC)C3] | 34 |
| 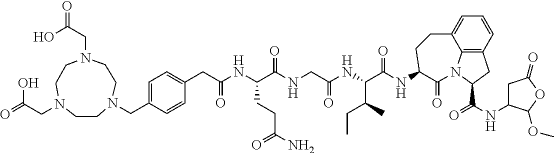 [SMILES: CC[C@H](C)[C@H](NC(=O)CNC(=O)[C@H](CCC(N)=O)NC(=O)Cc1ccc(CN2CCN(CC(=O)O)CCN(CC(=O)O)CC2)cc1)C(=O)N[C@H]1CCc2cccc3c2N(C1=O)[C@H](C(=O)NC1CC(=O)OC1OC)C3] | 35 |
| 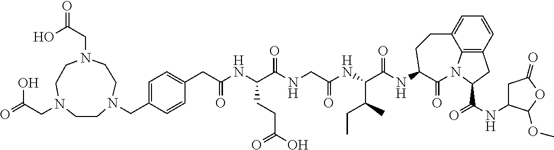 [SMILES: CC[C@H](C)[C@H](NC(=O)CNC(=O)[C@H](CCC(=O)O)NC(=O)Cc1ccc(CN2CCN(CC(=O)O)CCN(CC(=O)O)CC2)cc1)C(=O)N[C@H]1CCc2cccc3c2N(C1=O)[C@H](C(=O)NC1CC(=O)OC1OC)C3] | 36 |
| 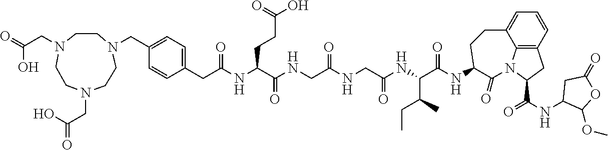 [SMILES: CC[C@H](C)[C@H](NC(=O)CNC(=O)CNC(=O)[C@H](CCC(=O)O)NC(=O)Cc1ccc(CN2CCN(CC(=O)O)CCN(CC(=O)O)CC2)cc1)C(=O)N[C@H]1CCc2cccc3c2N(C1=O)[C@H](C(=O)NC1CC(=O)OC1OC)C3] | 37 |
| 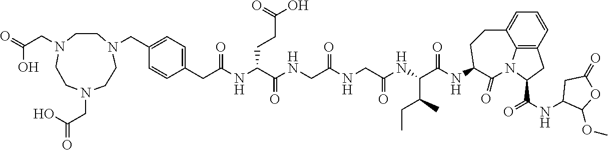 [SMILES: CC[C@H](C)[C@H](NC(=O)CNC(=O)CNC(=O)[C@@H](CCC(=O)O)NC(=O)Cc1ccc(CN2CCN(CC(=O)O)CCN(CC(=O)O)CC2)cc1)C(=O)N[C@H]1CCc2cccc3c2N(C1=O)[C@H](C(=O)NC1CC(=O)OC1OC)C3] | 38 |
| 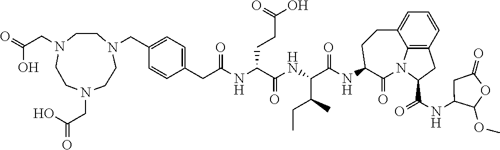 [SMILES: CC[C@H](C)[C@H](NC(=O)[C@@H](CCC(=O)O)NC(=O)Cc1ccc(CN2CCN(CC(=O)O)CCN(CC(=O)O)CC2)cc1)C(=O)N[C@H]1CCc2cccc3c2N(C1=O)[C@H](C(=O)NC1CC(=O)OC1OC)C3] | 39 |
| 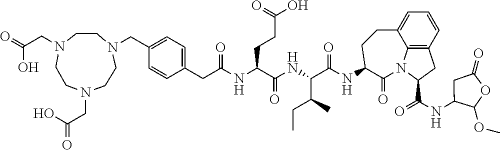 [SMILES: CC[C@H](C)[C@H](NC(=O)[C@H](CCC(=O)O)NC(=O)Cc1ccc(CN2CCN(CC(=O)O)CCN(CC(=O)O)CC2)cc1)C(=O)N[C@H]1CCc2cccc3c2N(C1=O)[C@H](C(=O)NC1CC(=O)OC1OC)C3] | 40 |
| 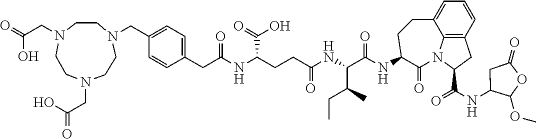 [SMILES: CC[C@H](C)[C@H](NC(=O)CC[C@H](NC(=O)Cc1ccc(CN2CCN(CC(=O)O)CCN(CC(=O)O)CC2)cc1)C(=O)O)C(=O)N[C@H]1CCc2cccc3c2N(C1=O)[C@H](C(=O)NC1CC(=O)OC1OC)C3] | 41 |
| 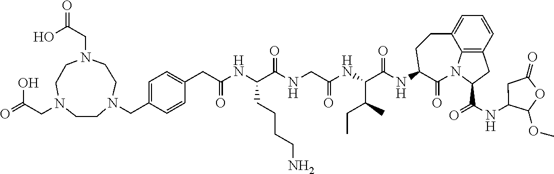 [SMILES: CC[C@H](C)[C@H](NC(=O)CNC(=O)[C@H](CCCCN)NC(=O)Cc1ccc(CN2CCN(CC(=O)O)CCN(CC(=O)O)CC2)cc1)C(=O)N[C@H]1CCc2cccc3c2N(C1=O)[C@H](C(=O)NC1CC(=O)OC1OC)C3] | 42 |
| 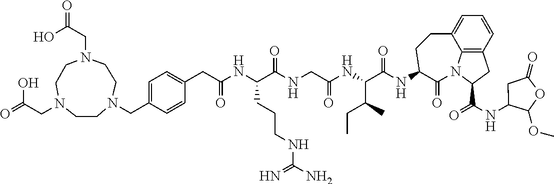 [SMILES: CC[C@H](C)[C@H](NC(=O)CNC(=O)[C@H](CCCNC(=N)N)NC(=O)Cc1ccc(CN2CCN(CC(=O)O)CCN(CC(=O)O)CC2)cc1)C(=O)N[C@H]1CCc2cccc3c2N(C1=O)[C@H](C(=O)NC1CC(=O)OC1OC)C3] | 43 |
| 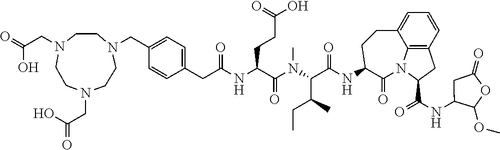 [SMILES: CC[C@H](C)[C@@H](C(=O)N[C@H]1CCc2cccc3c2N(C1=O)[C@H](C(=O)NC1CC(=O)OC1OC)C3)N(C)C(=O)[C@H](CCC(=O)O)NC(=O)Cc1ccc(CN2CCN(CC(=O)O)CCN(CC(=O)O)CC2)cc1] | 44 |
| 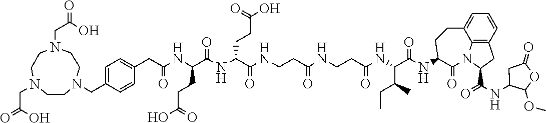 [SMILES: CC[C@H](C)[C@H](NC(=O)CCNC(=O)CCNC(=O)[C@@H](CCC(=O)O)NC(=O)[C@@H](CCC(=O)O)NC(=O)Cc1ccc(CN2CCN(CC(=O)O)CCN(CC(=O)O)CC2)cc1)C(=O)N[C@H]1CCc2cccc3c2N(C1=O)[C@H](C(=O)NC1CC(=O)OC1OC)C3] | 45 |
| 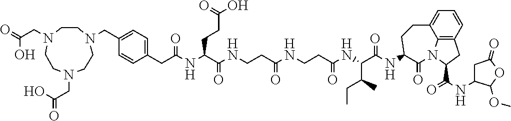 [SMILES: CC[C@H](C)[C@H](NC(=O)CCNC(=O)CCNC(=O)[C@H](CCC(=O)O)NC(=O)Cc1ccc(CN2CCN(CC(=O)O)CCN(CC(=O)O)CC2)cc1)C(=O)N[C@H]1CCc2cccc3c2N(C1=O)[C@H](C(=O)NC1CC(=O)OC1OC)C3] | 46 |

In some examples, the compound of Formula (I) is Compound 29, 30, 31, 32, 33, 34, 35, 36, 37, 38, 39, 40, 41, 42, 43, 44, 45, or 46. In another specific example, the compound of Formula (I) is Compound 29.

In some examples of Formula (Ia), A is NOTA, B is phenyl, Z is —CH₂—, and R³ is methyl, as in compounds of Formula (Id):

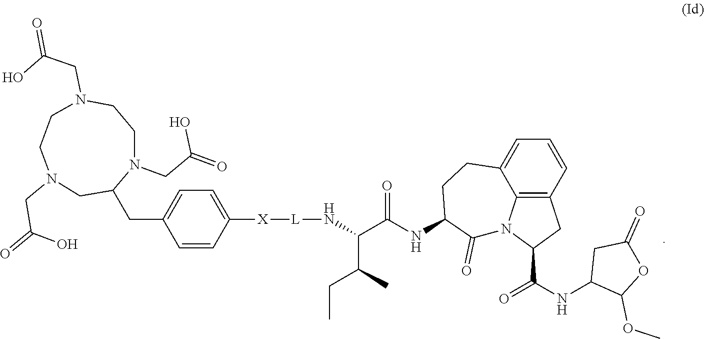

Exemplary compounds of Formula (Id) include those listed in Table 6.

TABLE 6
Exemplary Compounds of Formula (Id)

| Compounds | ID |
|---|---|
| 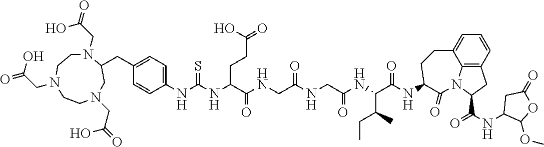 [SMILES: CC[C@H](C)[C@H](NC(=O)CNC(=O)CNC(=O)C(CCC(=O)O)NC(=S)Nc1ccc(CC2CN(CC(=O)O)CCN(CC(=O)O)CCN2CC(=O)O)cc1)C(=O)N[C@H]1CCc2cccc3c2N(C1=O)[C@H](C(=O)NC1CC(=O)OC1OC)C3] | 47 |
| 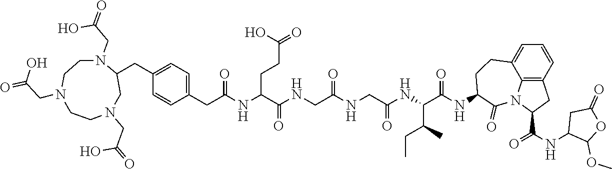 [SMILES: CC[C@H](C)[C@H](NC(=O)CNC(=O)CNC(=O)C(CCC(=O)O)NC(=O)Cc1ccc(CC2CN(CC(=O)O)CCN(CC(=O)O)CCN2CC(=O)O)cc1)C(=O)N[C@H]1CCc2cccc3c2N(C1=O)[C@H](C(=O)NC1CC(=O)OC1OC)C3] | 48 |
| 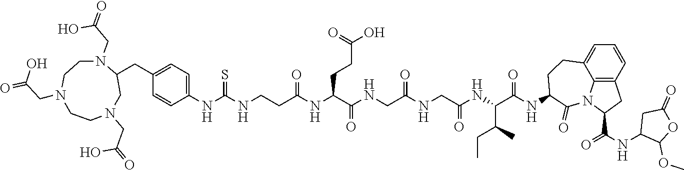 [SMILES: CC[C@H](C)[C@H](NC(=O)CNC(=O)CNC(=O)[C@H](CCC(=O)O)NC(=O)CCNC(=S)Nc1ccc(CC2CN(CC(=O)O)CCN(CC(=O)O)CCN2CC(=O)O)cc1)C(=O)N[C@H]1CCc2cccc3c2N(C1=O)[C@H](C(=O)NC1CC(=O)OC1OC)C3] | 49 |

In some examples, the compound of Formula (I) is Compound 47, 48, or 49.

### B. Compounds of Formula (II)

In some embodiments, the disclosure provides for compounds of Formula (II), shown below. Compared to the Formula (I) compounds disclosed herein, the Formula (II) compounds further contain a metal, which may be conjugated to a radioisotope, via the chelating moiety A.

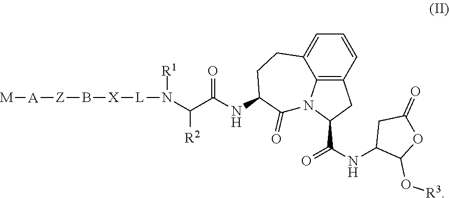

In Formula (II), M is a metal, or a metal linked to a radioisotope. Suitable metals for use in the present disclosure include those which are useful in imaging Granzyme B, for instance metals that are suitable radioimaging agents, as well as metals that can bind non-metal radioisotopes which are suitable radioimaging agents. An exemplary metal radioisotope is ⁶⁸Ga. An exemplary non-metallic radioisotope is ¹⁸F, which may be conjugated with Al for loading into the Granzyme B binding compounds disclosed herein

Each of A, X, Y. Z. L, R¹, R² and R³ is as defined herein. See, e.g., the section titled Compounds of Formula (I) above.

In some embodiments, R¹ is H, and R² is C₄ alkyl as in compounds of Formula (IIa):

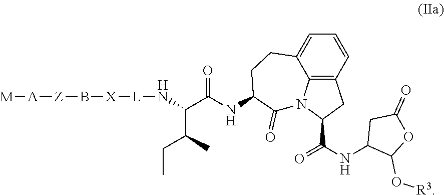

In some specific examples, X is —CH₂C(O)—, as in compounds of Formula (IIb):

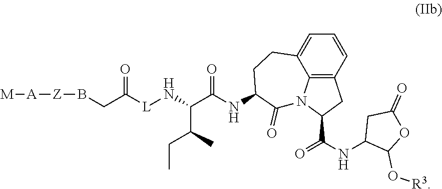

In some examples, R³ is methyl, as in compounds of Formula (IIc):

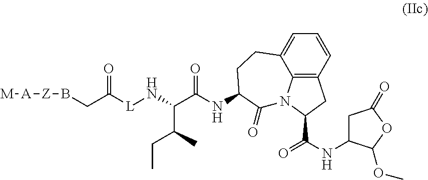

In some examples of Formula (IIc), B is piperidyl, and Z is —CH₂— as in compounds of Formula (Ic-A):

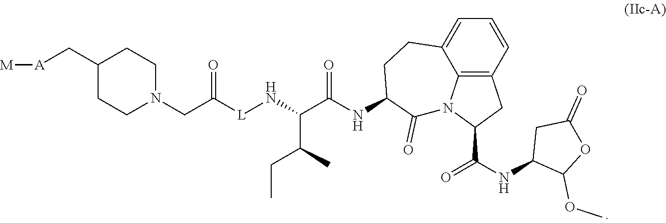

Exemplary compounds of Formula (IIc-A) include those listed in Table 7.

TABLE 7
Exemplary Compounds of Formula (IIc-A)

| Compounds | ID |
|---|---|
| 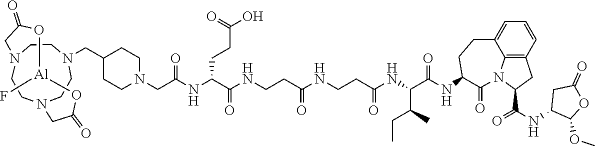 | 1-A1 |
| 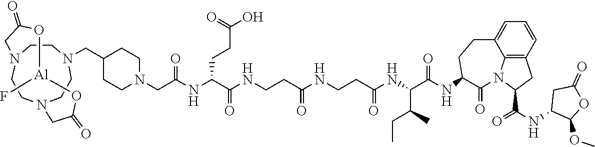 | 2-A1 |
| 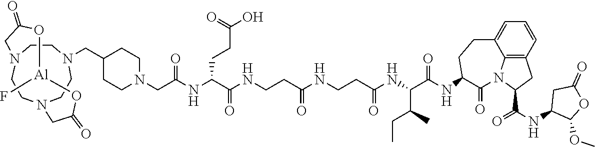 | 3-A1 |
| 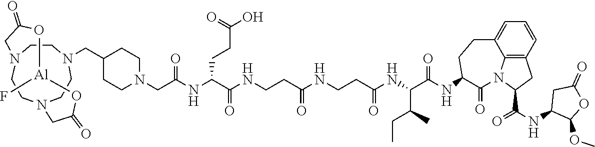 | 4-A1 |
| 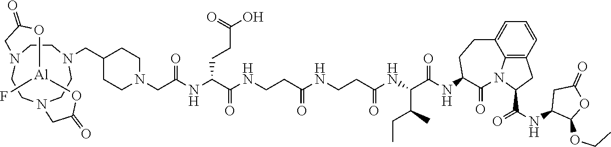 | 5-A1 |
| 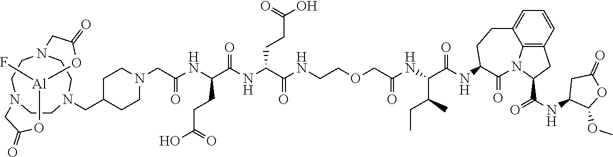 | 6-A1 |
| 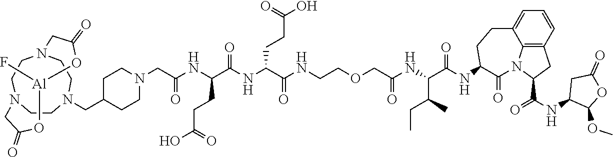 | 7-A1 |
| 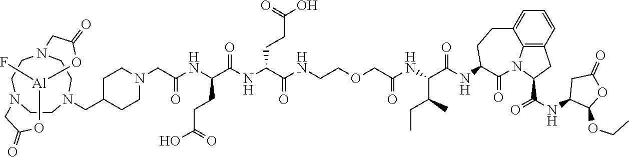 | 8-A1 |
| 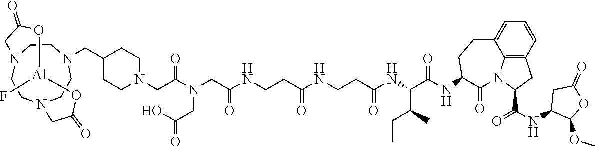 | 9-A1 |
| 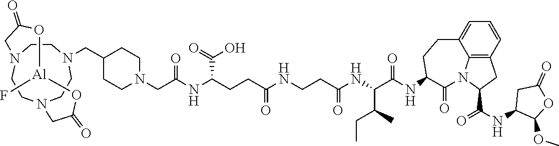 | 10-A1 |
| 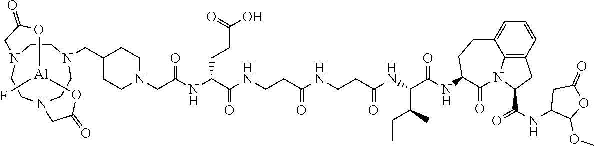 | 11-A1 |
| 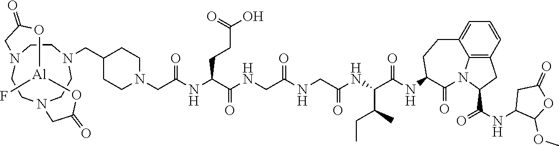 | 12-A1 |
| 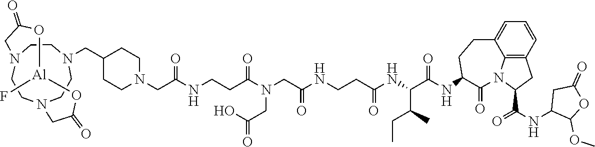 | 13-A1 |
| 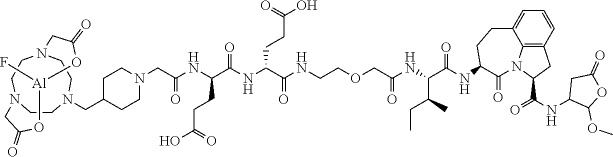 | 14-A1 |
| 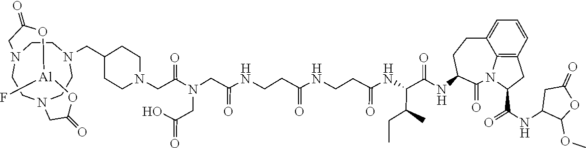 | 15-A1 |
| 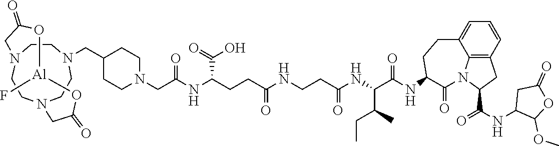 | 16-A1 |
| 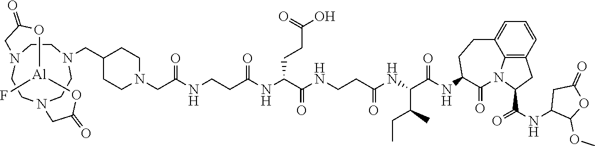 | 17-A1 |

In some examples, the compound of Formula (I) is Compound 1-Al, 2-Al, 3-Al, 4-Al, 5-Al, 6-Al, 7-Al, 8-Al, 9-Al, 10-Al, 11-Al, 12-Al, 13-Al, 14-Al, 15-Al, 16-Al, or 17-Al. In one specific example, the compound of Formula (I) is Compound 4-Al. In another specific example, the compound of Formula (I) is Compound 7-Al. As described herein, the above examples benefit from the piperidine ring, which exhibits improved properties over other related moieties.

In some examples of Formula (IIc), B is piperazyl, and Z is —CH₂C(O)— as in compounds of Formula (IIc-B):

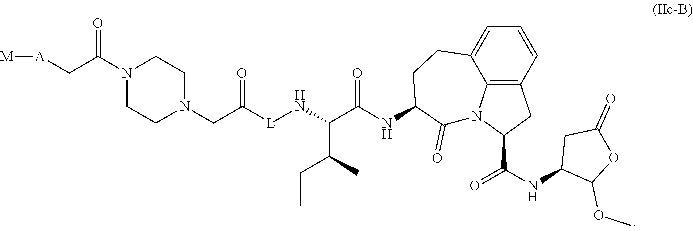

Exemplary compounds of Formula (IIc-B) include those listed in Table 8.

TABLE 8
Exemplary Compounds of Formula (Ic-B)

| Compounds | ID |
|---|---|
| 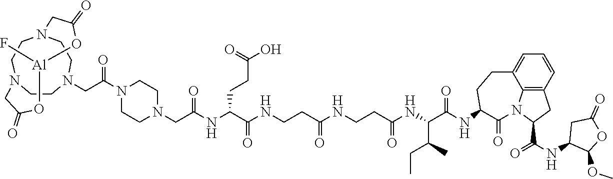 | 18-A1 |
| 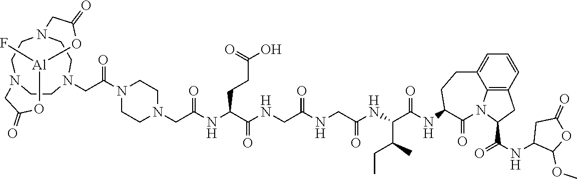 | 19-A1 |
| 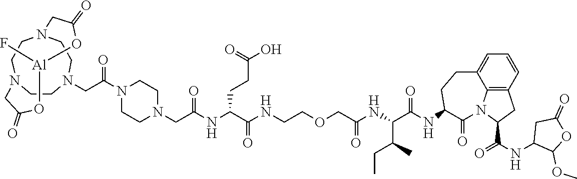 | 20-A1 |
| 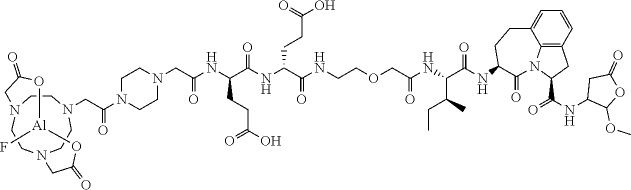 | 21-A1 |
| 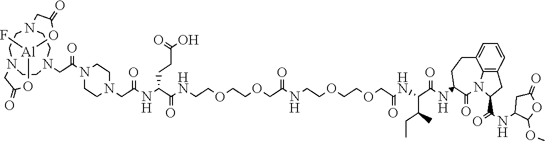 | 22-A1 |
| 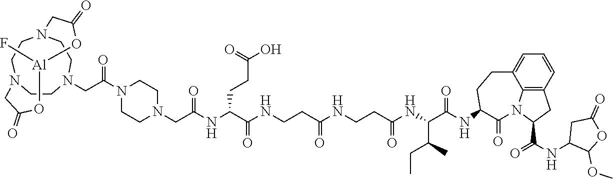 | 23-A1 |
| 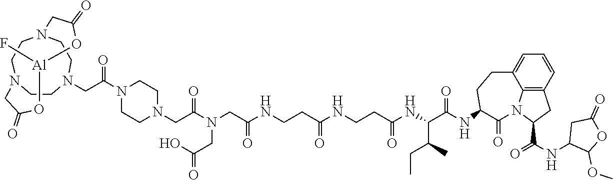 | 24-A1 |
| 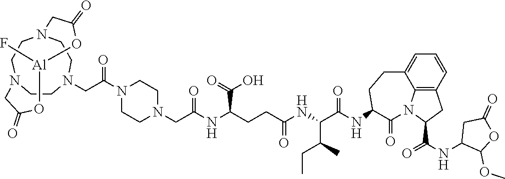 | 25-A1 |
| 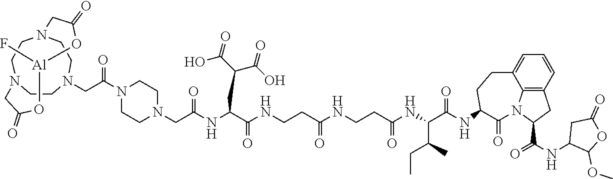 | 26-A1 |
| 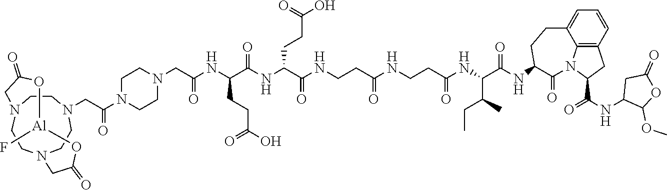 | 27-A1 |
| 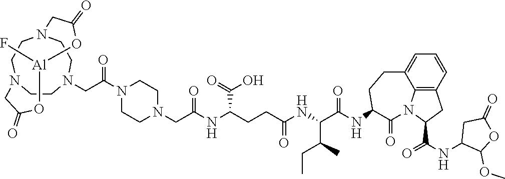 | 28-A1 |

In some examples, the compound of Formula (I) is Compound 18-Al, 19-Al, 20-Al, 21-Al, 22-Al, 23-Al, 24-Al, 25-Al, 26-Al, 27-Al, or 28-Al. As described herein, the above examples benefit from the piperazine ring, which exhibits improved properties over some other related moieties.

In some examples of Formula (IIc), B is phenyl, and Z is —CH₂— as in compounds of Formula (IIc-C):

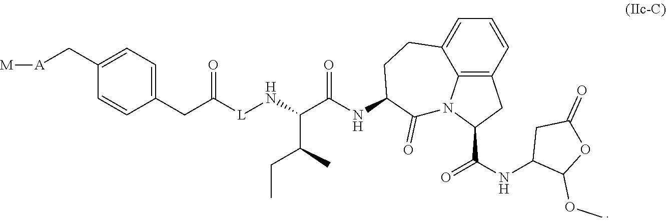

Exemplary compounds of Formula (IIc-C) include those listed in Table 9.

TABLE 9
Exemplary Compounds of Formula (IIc-C)

| Compounds | ID |
|---|---|
| 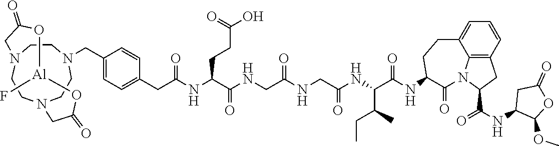 | 29-A1 |
| 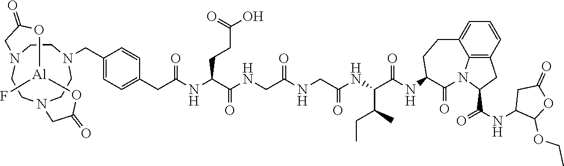 | 30-A1 |
| 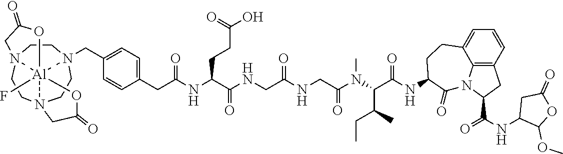 | 31-A1 |
| 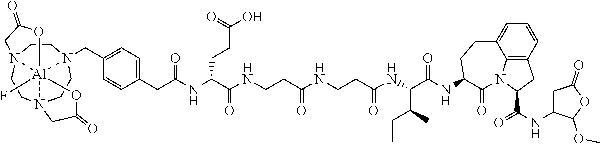 | 32-A1 |
| 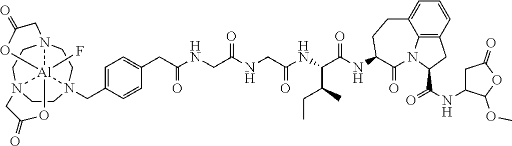 | 33-A1 |
| 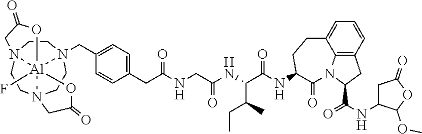 | 34-A1 |
| 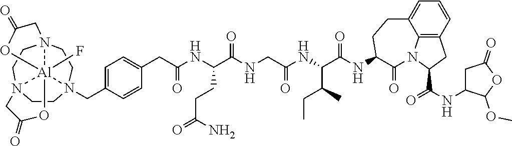 | 35-A1 |
| 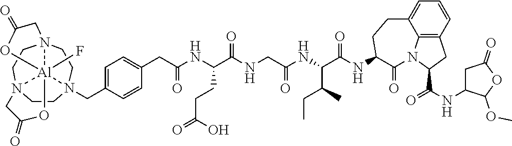 | 36-A1 |
| 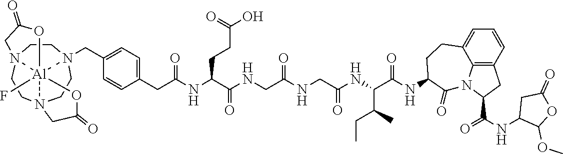 | 37-A1 |
| 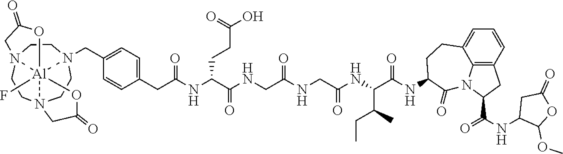 | 38-A1 |
| 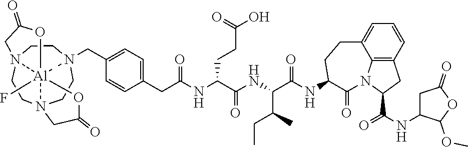 | 39-A1 |
| 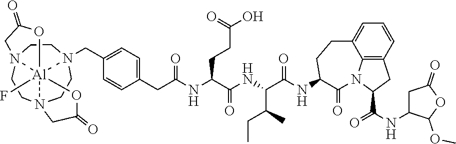 | 40-A1 |
| 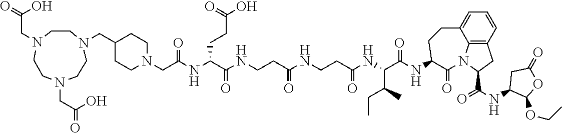 [SMILES: CCO[C@@H]1OC(=O)C[C@@H]1NC(=O)[C@@H]1Cc2cccc3c2N1C(=O)[C@@H](NC(=O)[C@@H](NC(=O)CCNC(=O)CCNC(=O)[C@@H](CCC(=O)O)NC(=O)CN1CCC(CN2CCN(CC(=O)O)CCN(CC(=O)O)CC2)CC1)[C@@H](C)CC)CC3] | 41-A1 |
| 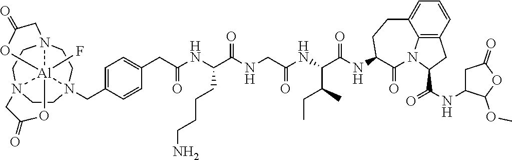 | 42-A1 |
| 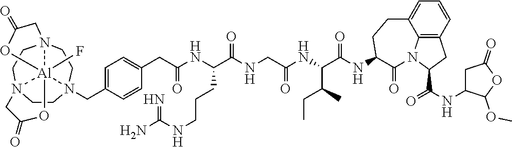 | 43-A1 |
| 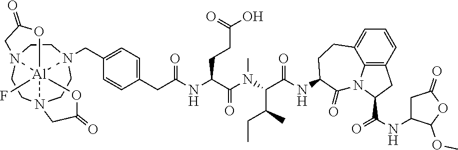 | 44-A1 |
| 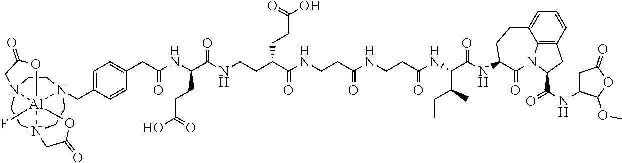 | 45-A1 |
| 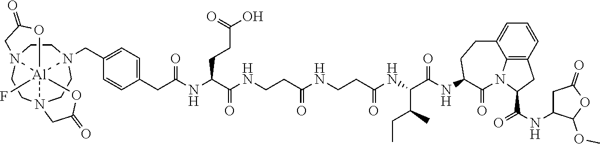 | 46-A1 |

In some examples, the compound of Formula (I) is Compound 29-Al, 30-Al, 31-Al, 32-Al, 33-Al, 34-Al, 35-Al, 36-Al, 37-Al, 38-Al, 39-Al, 40-Al, 41-Al, 42-Al, 43-Al, 44-Al, 45-Al, or 46-Al. In another specific example, the compound of Formula (I) is Compound 29 in some examples of Formula (IIa), A is NOTA, B is phenyl, Z is —CH₂—, and R³ is methyl, as in compounds of Formula (IId):

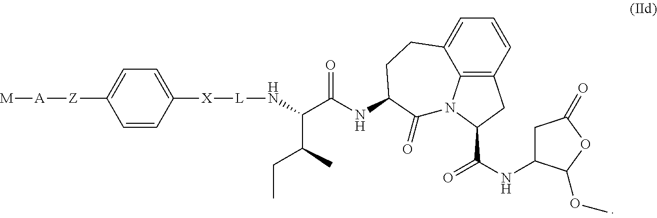

Exemplary compounds of Formula (IId) include those listed in Table 10.

TABLE 10
Exemplary Compounds of Formula (Id)

| Compounds | ID |
|---|---|
| 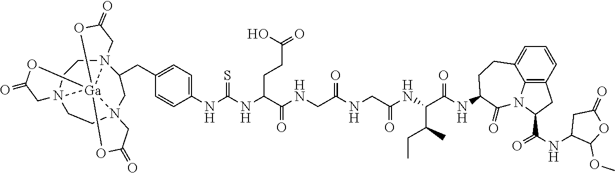 | 47-Ga |
| 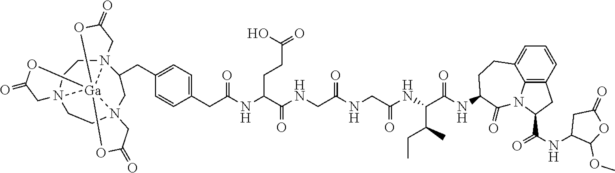 | 48-Ga |
| 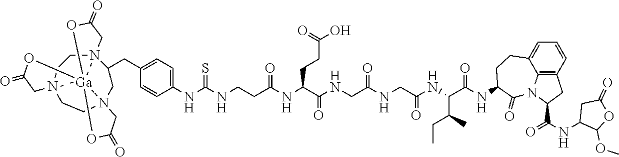 | 49-Ga |

In some examples, the compound of Formula (I) is Compound 47-Al, 48-Al, or 49-Al.

As shown in Examples below, the prodrugs of GZB-binding compounds disclosed herein, comprising methyl or ethyl ethers, as well as specific peptide linker structures, showed one or more of the following favorable features: production of a single isomer, synthesis with reliable stereochemical outcome, and facile liberation of the active drug in vivo.

The above compounds, when containing a radioisotope, are useful as prodrugs which can be converted in vivo into useful imaging agents in one or more of the methods provided herein. In addition, the radioisotope-containing prodrugs provided herein can be converted in vivo into compounds that may also be useful in one or more therapeutic applications, when administered to a subject in a therapeutically effective amount. For example, the above compounds containing ¹⁸F may be useful as imaging agents (e.g., as non-toxic and/or non-therapeutic radioisotopes) when administered to the subject at low concentrations (e.g., 5 mCi). In some embodiments the isotope can be toxic. As pointed out above, the present application also includes pharmaceutically acceptable salts of the compounds described herein. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

### C. Chemical Synthesis of Prodrugs of Granzyme B-Targeting Compounds

As will be appreciated, the compounds provided herein, including stereoisomers and salts thereof, can be prepared using known organic synthesis techniques and can be synthesized according to any of numerous possible synthetic routes.

The compounds disclosed herein, or a pharmaceutically acceptable salt thereof, can be prepared by following the exemplary protocols described below. Appropriate protective groups for use in such syntheses are known in the field. See, e.g., McOmie, Protective Groups in Organic Chemistry, (1973):98.

General synthetic procedures, and working examples thereof, for the preparation of peptide linker L, the Fmoc-Haic(2S, 5S)—OH tricycle moiety, and the appropriate metal complexation with the chelating moiety, can be found in International Application No.: PCT/US2021/036661, filed on Jun. 9, 2021, the relevant disclosures of which are incorporated by reference for the subject matter and purpose referenced herein.

The chelating moiety can be prepared and bound to the peptide linker L by appropriate means known in the art.

The prodrug moiety can be prepared using synthetic means known in the art. In a preferred embodiment, the enantiopure α-amino alcohol of reduced aspartic acid is oxidized to the aldehyde and formed into an acetal of choice. The γ-carboxylate can then condense onto the acetal forming the ring. The resulting diastereomers can then be separated. Use of natural or unnatural aspartic acid affords access to all four diastereomers.

Many appropriate imaging agents (e.g., radioisotopes) are known in the art (see, for e.g., U.S. Pat. Nos. 5,021,236; 4,938,948; and 4,472,509, the disclosure of each of which is incorporated herein by reference in its entirety). Radioactively labeled compounds, or a pharmaceutically acceptable salt thereof, provided herein may be prepared according to well-known methods in the art. Synthetic methods for incorporating radioisotopes into organic compounds are well known in the art, and one of ordinary skill in the art will readily recognize other methods applicable for the compounds provided herein.

It will be appreciated by one skilled in the art that the processes described herein are not the exclusive means by which compounds provided herein may be synthesized and that a broad repertoire of synthetic organic reactions is available to be potentially employed in synthesizing compounds provided herein. The person skilled in the art knows how to select and implement appropriate synthetic routes. Suitable synthetic methods of starting materials, intermediates and products may be identified by reference to the literature, including reference sources such as: Advances in Heterocyclic Chemistry, Vols. 1-107 (Elsevier, 1963-2012); Journal of Heterocyclic Chemistry Vols. 1-49 (Journal of Heterocyclic Chemistry, 1964-2012); Carreira, et al. (Ed.) Science of Synthesis, Vols. 1-48 (2001-2010) and Knowledge Updates KU2010/1-4; 2011/1-4; 2012/1-2 (Thieme, 2001-2012); Katritzky, et al. (Ed.) Comprehensive Organic Functional Group Transformations, (Pergamon Press, 1996); Katritzky et al. (Ed.); Comprehensive Organic Functional Group Transformations II (Elsevier, 2^{nd} Edition, 2004); Katritzky et al. (Ed.), Comprehensive Heterocyclic Chemistry (Pergamon Press, 1984); Katritzky et al., Comprehensive Heterocyclic Chemistry II, (Pergamon Press, 1996); Smith et al., March's Advanced Organic Chemistry: Reactions, Mechanisms, and Structure, 6^{th} Ed. (Wiley, 2007); Trost et al. (Ed.), Comprehensive Organic Synthesis (Pergamon Press, 1991).

The reactions for preparing compounds described herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, (e.g., temperatures which can range from the solvent's freezing temperature to the solvent's boiling temperature). A given reaction can be carried out in one solvent or a mixture of more than one solvent. Depending on the particular reaction step, suitable solvents for a particular reaction step can be selected by the skilled artisan.

Preparation of the compounds described herein can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) and normal phase silica chromatography.

### II. Pharmaceutical Compositions

Any of the compounds of Formula (I) and Formula (II), or a pharmaceutically acceptable salt thereof, may be mixed with a pharmaceutically acceptable carrier to form a pharmaceutical composition for use in Granzyme B imaging and/or for therapeutic purposes as disclosed herein. In some embodiments, provided herein pharmaceutical compositions comprising, as the active ingredient, a compound with a metal as provided herein (a Formula (II) compound), or a pharmaceutically acceptable salt thereof, in combination with one or more pharmaceutically acceptable carriers (excipients). “Acceptable” means that the carrier must be compatible with the active ingredient of the composition (and preferably, capable of stabilizing the active ingredient) and not deleterious to the subject to be treated. Suitable carriers include microcrystalline cellulose, mannitol, glucose, defatted milk powder, polyvinylpyrrolidone, and starch, or a combination thereof.

Some examples of suitable excipients include, without limitation, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The pharmaceutical formulations can additionally include, without limitation, lubricating agents such as talc, magnesium stearate, and mineral oil; wetting agents; emulsifying and suspending agents; preserving agents such as methyl- and propylhydroxy-benzoates; sweetening agents; flavoring agents, or combinations thereof. See Remington's Pharmaceutical Sciences, 17^{th} ed., Mack Publishing Company, Easton, Pa., 1985, p. 1418 for more information on acceptable pharmaceutical compositions.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. Parenteral administration can be in the form of a single bolus dose, or may be, for example, by a continuous perfusion pump. In addition, it can be administered to the subject via injectable depot routes of administration such as using 1-, 3-, or 6-month depot injectable or biodegradable materials and methods.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipients is infused. Physiologically acceptable excipients may include, for example, 5% dextrose, 0.9% saline, Ringer's solution, or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

For oral administration, the composition can take the form of, for example, tablets or capsules, prepared by conventional means with acceptable excipients such as binding agents (for example, pre-gelatinised maize starch, polyvinylpyrrolidone or hydroxypropyl methylcellulose); fillers (for example, lactose, microcrystalline cellulose or calcium hydrogen phosphate); lubricants (for example, magnesium stearate, talc or silica); disintegrants (for example, potato starch or sodium starch glycolate); or wetting agents (for example, sodium lauryl sulphate). The tablets can be coated by methods well known in the art.

In some embodiments, the above compounds, or a pharmaceutically acceptable salt thereof, provided herein are suitable for parenteral administration. In some embodiments, the above compounds, or a pharmaceutically acceptable salt thereof, are suitable for intravenous administration.

Pharmaceutical compositions and formulations for topical administration may include transdermal patches, ointments, lotions, creams, gels, drops, suppositories, sprays, liquids, and powders. Conventional pharmaceutical carriers, aqueous, powder or oily bases, thickeners and the like may be necessary or desirable.

In making the pharmaceutical compositions provided herein, the active ingredient is typically mixed with an excipient, diluted by an excipient, or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient.

Thus, the pharmaceutical compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders.

### III. Methods of Use

The present application further provides a method of imaging Granzyme B using one of the above-described prodrug compounds, or a pharmaceutically acceptable salt thereof. In some embodiments, the method is an in vitro method. In some embodiments, the method is an in vivo method.

Alternatively, the method of imaging as disclosed herein may be an in vivo imaging method, comprising administering the GZB-binding compound disclosed herein or a pharmaceutical composition comprising such to a subject in need thereof via a suitable route, for example, intravenous injection or local injection.

As used herein, the term “subject,” refers to any animal, including mammals and invertebrates. For example, mice, rats, other rodents, rabbits, dogs, cats, swine, cattle, sheep, horses, primates, fish, and humans. In some embodiments, the subject is a human. In some embodiments, the subject is a mouse. In some embodiments, the subject is a fish (e.g., a zebra fish).

The present application further provides a method of imaging Granzyme B in a cell or tissue, comprising:
i) contacting the cell or tissue with an effective amount of one of the above compounds, or a pharmaceutically acceptable salt thereof, and
ii) imaging the cell or tissue with a suitable imaging technique, thereby imaging Granzyme B in the cell or tissue.

The present application further provides a method of imaging Granzyme B in a sample, cell sample or tissue sample, comprising:
i) contacting the sample, cell sample or tissue sample with effective amount of one of the above compounds, or a pharmaceutically acceptable salt thereof, and
ii) imaging the sample, cell sample or tissue sample with a suitable imaging technique, thereby imaging Granzyme B in the sample, cell sample or tissue sample.

As used herein within the context of imaging, the term “sample” refers to a biological sample other than cell or tissue sample which is obtained from a subject. For example, the sample includes but not limited to saliva, blood, and urine.

The present application further provides a method of imaging Granzyme B in a subject, comprising:
i) administering to the subject an effective amount of one of the above compounds, or a pharmaceutically acceptable salt thereof, and
ii) imaging the subject with a suitable imaging technique, thereby imaging Granzyme B in the subject.

The present application further provides a method of imaging an immune response in a cell or tissue sample, comprising:
i) contacting the cell or tissue sample with an effective amount of one of the above compounds, or a pharmaceutically acceptable salt thereof, and
ii) imaging the cell or tissue sample with a suitable imaging technique, thereby imaging the immune response in the cell or tissue sample.

The present application further provides a method of imaging an immune response in a subject, comprising:
i) administering to the subject an effective amount of one of the above compounds, or a pharmaceutically acceptable salt thereof, and
ii) imaging the subject with a suitable imaging technique, thereby imaging the immune response in the subject.

The present application further provides a method of monitoring treatment of a disease in a subject, comprising:
i) administering to the subject an effective amount of one of the above compounds, or a pharmaceutically acceptable salt thereof, and
ii) imaging the subject with a suitable imaging technique.

The present application further provides a method of monitoring an immune response in the treatment of a disease in a subject, comprising:
i) administering to the subject an effective amount of one of the above compounds, or a pharmaceutically acceptable salt thereof, and
ii) imaging the subject with a suitable imaging technique.

In some embodiments, the methods provided herein further comprise waiting a time sufficient to allow the compound, or a pharmaceutically acceptable salt thereof, to accumulate at a cell or tissue site (e.g., a cell or tissue site in a subject) associated with the disease, prior to imaging.

In some embodiments, the methods provided herein further comprise waiting a time sufficient to allow the compound, or a pharmaceutically acceptable salt thereof, to bind Granzyme B at a cell or tissue site (e.g., a cell or tissue site in a subject) associated with the disease, prior to imaging.

In some embodiments, the time sufficient is from about 30 seconds to about 24 hours, for example, about 30 seconds to about 24 hours, about 30 seconds to about 12 hours, about 30 seconds to about 6 hours, about 30 seconds to about 2 hours, about 30 seconds to about 1 hour, about 30 seconds to about 30 minutes, about 30 seconds to about 10 minutes, about 10 minutes to about 24 hours, about 10 minutes to about 12 hours, about 10 minutes to about 6 hours, about 10 minutes to about 2 hours, about 10 minutes to about 1 hour, about 10 minutes to about 30 minutes, about 30 minutes to about 24 hours, about 30 minutes to about 12 hours, about 30 minutes to about 6 hours, about 30 minutes to about 2 hours, about 30 minutes to about 1 hour, about 1 hour to about 24 hours, about 1 hour to about 12 hours, about 1 hour to about 6 hours, about 1 hour to about 2 hours, about 2 hours to about 24 hours, about 2 hours to about 12 hours, about 2 hours to about 6 hours, about 6 hours to about 24 hours, about 6 hours to about 12 hours, or about 12 hours to about 24 hours.

In some embodiments, the suitable imaging technique is a non-invasive imaging technique. In some embodiments, the suitable imaging technique is a minimally invasive imaging technique. As used herein, the term “minimally invasive imaging technique” comprises imaging techniques employing the use of an internal probe or injection of one of the above compounds, or a pharmaceutically acceptable salt thereof, or radiotracer via syringe.

Exemplary imaging techniques include, but are not limited to, fluoroscopic imaging, x-ray imaging, magnetic resonance imaging (MRI), ultrasound imaging, photoacoustic imaging, thermographic imaging, tomographic imaging, echocardiographic imaging, positron emission tomography (PET) imaging, PET with computed tomography (CT) imaging, PET-MRI, single-photon emission computed tomography (SPECT), and ultrasound imaging. In some embodiments, the suitable imaging technique is selected from the group consisting of PET imaging, PET-CT, PET-MRI, and SPECT.

In some embodiments, the suitable imaging technique is selected from the group consisting of PET imaging, PET with computed tomography imaging, and PET with magnetic resonance imaging (MRI). In some embodiments, the suitable imaging technique is selected PET imaging.

The results from any of the Granzyme B imaging methods disclosed herein may be relied on for diagnostic and/or prognostic purposes. In some embodiments, the results can be relied on to identify a patient suitable for treatment of an immunoregulatory abnormality (disease) with a therapeutic agent (e.g., an anti-inflammatory agents, steroids, immunotherapy agents, chemotherapeutic agents, or therapeutic antibodies). In other embodiments, the results can be relied on for monitoring efficacy of a therapeutic agent such as those provided herein. For example, presence of Granzyme B or an increase of the GZB level may indicate that the patient is suitable and/or responsive to the therapeutic agent. In that case, the method disclosed herein may further comprise administering the therapeutic agent to the patient for treating the target disease.

In some embodiments, a disease as described herein is selected from the group consisting of an autoimmune disorder, an inflammatory disorder, a skin disorder, cancer, and a cardiovascular disorder. As used herein, the term “disease” is used interchangeable with the term “immunoregulatory abnormality.”

In some embodiments, the disease is a cancer. In some embodiments, the cancer comprises a solid tumor. In some embodiments, the cancer is a hematological cancer (e.g., leukemia, lymphoma, and the like). Exemplary solid cancers include, but are not limited to, brain, breast cancer, cervical cancer, colorectal cancer, lung cancer, lymphoma, melanoma, bladder cancer, renal cell carcinoma, multiple myeloma, pancreatic cancer, and prostate cancer. Exemplary hematological cancers include, but are not limited to, Hairy-cell leukemia, Kaposi's sarcoma, follicular lymphoma, chronic myeloid leukemia, cutaneous T-cell lymphoma, peripheral T-cell lymphoma, T-cell prolymphocytic leukemia, Classical Hodgkin's lymphoma, B-cell non-Hodgkin's lymphoma, chronic lymphocytic leukemia, acute myeloid leukemia, myelodysplastic syndrome, primary myelofibrosis, post-essential thrombocythemia myelofibrosis, post-polycythemia vera myelofibrosis. Other examples include melanoma, renal cell carcinoma, prostate cancer, non-small cell lung cancer, small cell lung cancer, glioblastoma, hepatocellular carcinoma, urothelial carcinoma, esophageal carcinoma, gastroesophageal carcinoma, gastric cancer, multiple myeloma, colon cancer, rectal cancer, squamous cell carcinoma of the head and neck, epithelial ovarian cancer (EOC), primary peritoneal cancer, fallopian tube carcinoma, HER2+ breast cancer, ER+/PR+/HER2− breast cancer, triple-negative breast cancer, gastric cancer, pancreatic cancer, bladder cancer, Merkel cell cancer, nasopharyngeal cancer, adrenocortical carcinoma, meningioma, neuroblastoma, retinoblastoma, osteosarcoma, rhabdomyosarcoma, Ewing's sarcoma, liposarcoma, fibrosarcoma, leiomyosarcoma, peripheral primitive neuroectodermal tumor, squamous cell carcinoma of the cervix, squamous cell carcinoma of the vagina, and squamous cell carcinoma of the vulva. In some examples, the cancer is colon cancer.

Additional exemplary immunoregulatory abnormalities include, but are not limited to, graft-versus-host disease, rheumatoid arthritis, systemic lupus erythematosus, Hashimoto's thyroiditis, multiple sclerosis, myasthenia gravis, type I diabetes, uveitis, posterior uveitis, allergic encephalomyelitis, glomerulonephritis, rheumatic fever, post-infectious glomerulonephritis, psoriasis, atopic dermatitis, contact dermatitis, eczematous dermatitis, seborrhoeic dermatitis, lichen planus, pemphigus, bullous pemphigoid, epidermolysis bullosa, urticaria, angioedemas, vasculitis, erythema, cutaneous eosinophilia, lupus erythematosus, acne, alopecia areata, keratoconjunctivitis, vernal conjunctivitis, uveitis associated with Behcet's disease, keratitis, herpetic keratitis, conical cornea, dystrophia epithelialis corneae, corneal leukoma, ocular pemphigus, Mooren's ulcer, scleritis, Graves' opthalmopathy, Vogt-Koyanagi-Harada syndrome, sarcoidosis, pollen allergies, reversible obstructive airway disease, bronchial asthma, allergic asthma, intrinsic asthma, extrinsic asthma, dust asthma, chronic or inveterate asthma, late asthma and airway hyper-responsiveness, bronchitis, gastric ulcers, vascular damage caused by ischemic diseases and thrombosis, ischemic bowel diseases, inflammatory bowel diseases, necrotizing enterocolitis, intestinal lesions associated with thermal burns, coeliac diseases, proctitis, eosinophilic gastroenteritis, mastocytosis, Crohn's disease, ulcerative colitis, migraine, rhinitis, eczema, interstitial nephritis, Goodpasture's syndrome, hemolytic-uremic syndrome, diabetic nephropathy, multiple myositis, Guillain-Barre syndrome, Meniere's disease, polyneuritis, multiple neuritis, mononeuritis, radiculopathy, hyperthyroidism, Basedow's disease, pure red cell aplasia, aplastic anemia, hypoplastic anemia, idiopathic thrombocytopenic purpura, autoimmune hemolytic anemia, agranulocytosis, pernicious anemia, megaloblastic anemia, anerythroplasia, osteoporosis, sarcoidosis, fibroid lung, idiopathic interstitial pneumonia, dermatomyositis, leukoderma vulgaris, ichthyosis vulgaris, photoallergic sensitivity, cutaneous T cell lymphoma, arteriosclerosis, atherosclerosis, aortitis syndrome, polyarteritis nodosa, myocardosis, scleroderma, Wegener's granuloma, Sjogren's syndrome, adiposis, eosinophilic fascitis, lesions of gingiva, periodontium, alveolar bone, substantia ossea dentis, glomerulonephritis, male pattern alopecia, alopecia senilis by preventing epilation, alopecia senilis by providing hair germination and/or promoting hair generation and hair growth, muscular dystrophy, pyoderma, Sezary's syndrome, Addison's disease, ischemia-reperfusion injury of organs, transplantation disease, ischemic disease, endotoxin-shock, pseudomembranous colitis, colitis caused by drug or radiation, ischemic acute renal insufficiency, chronic renal insufficiency, toxinosis caused by lung-oxygen or drugs, lung cancer, pulmonary emphysema, cataracta, siderosis, retinitis pigmentosa, senile macular degeneration, vitreal scarring, corneal alkali burn, dermatitis erythema multiforme, linear IgA ballous dermatitis and cement dermatitis, gingivitis, periodontitis, sepsis, pancreatitis, aging, carcinogenesis, metastasis of carcinoma and hypobaropathy, histamine or leukotriene-C4 release associated diseases, Behcet's disease, autoimmune hepatitis, primary biliary cirrhosis, sclerosing cholangitis, partial liver resection, acute liver necrosis, necrosis caused by toxin, viral hepatitis, shock, anoxia, B-virus hepatitis, non-A/non-B hepatitis, cirrhosis, alcoholic cirrhosis, hepatic failure, fulminant hepatic failure, late-onset hepatic failure, acute-on-chronic liver failure, cytomegalovirus infection, HCMV infection, AIDS, senile dementia, trauma, chronic bacterial infection, malignancy of lymphoid origin, acute lymphocytic leukemia, chronic lymphocytic leukemia, acute lymphocytic lymphoma, and chronic lymphocytic lymphoma.

Exemplary autoimmune diseases include, but are not limited to, systemic lupus erythematosis, chronic rheumatoid arthritis, type I diabetes mellitus, inflammatory bowel disease, biliary cirrhosis, uveitis, multiple sclerosis, Crohn's disease, ulcerative colitis, bullous pemphigoid, sarcoidosis, psoriasis, autoimmune myositis, Wegener's granulomatosis, ichthyosis, Graves ophthalmopathy, asthma, schleroderma and Sjogren's syndrome.

In some embodiments, the disease can be bone marrow rejection, organ transplant rejection, and graft-versus-host disease.

When employed in methods of treating a disease, the above compounds of Formula (II), or pharmaceutically acceptable salts thereof, provided herein can be administered in combination with one or more additional therapeutic agents. In some instances, the additional therapeutic agent induces an immune response in a subject receiving the treatment. The Formula (II) compounds can be used to monitor such an immune response based on presence/absence of GZB or change of the GZB levels in the subject.

In some embodiments, a compound of Formula (II) may be given to a patient after the patient receives at least one dose of the additional therapeutic agent. Based on the GZB-imaging results arising from the compound of Formula (II) compound, the patient may continue the treatment comprising the additional therapeutic agent. In some examples, the dosing and/or dosing schedule of the additional therapeutic agent may be adjusted.

Examples of the additional therapeutic agents include, but are not limited to, anti-inflammatory agents, steroids, immunotherapy agents, chemotherapeutic agents, and therapeutic antibodies.

In some embodiments, the therapeutic agent is an antibody. Exemplary antibodies for use in a combination therapy include, but are not limited to, trastuzumab (e.g. anti-HER2), ranibizumab (e.g. anti-VEGF-A), bevacizumab (e.g. anti-VEGF), panitumumab (e.g. anti-EGFR), cetuximab (e.g. anti-EGFR), rituxan (anti-CD20), antibodies directed to c-MET, and antibody inhibitors of Granzyme B (e.g., Clone GB11, Clone GrB-7, and NCL-L-Gran-B), ipilimumab (anti-CTLA-4), nivolumab (anti-PD-1), pembrolizumab (anti-PD-1), atezolizumab (anti-PD-1), elotuzumab (anti-SLAM7), and daratumumab (anti-CD38).

In some embodiments, the additional therapeutic agent is a steroid. Exemplary steroids include corticosteroids such as cortisone, dexamethasone, hydrocortisone, methylprednisolone, prednisolone, and prednisone. In some embodiments, the additional therapeutic agent is a corticosteroid.

In some embodiments, the additional therapeutic agent is an anti-inflammatory compound. Exemplary anti-inflammatory compounds include aspirin, choline salicylates, celecoxib, diclofenac potassium, diclofenac sodium, diclofenac sodium with misoprostol, diflunisal, etodolac, fenoprofen, flurbiprofen, ibuprofen, ketoprofen, meclofenamate sodium, mefenamic acid, nabumetone, naproxen, naproxen sodium, oxaprozin, piroxican, rofecoxib, salsalate, sodium salicylate, sulindac, tolmetin sodium, and valdecoxib.

In some embodiments, the additional therapeutic agent is chemotherapeutic agent. Exemplary chemotherapeutic agents include, but are not limited to, a cytostatic agent, cisplatin, doxorubicin, taxol, etoposide, irinotecan, topotecan, paclitaxel, docetaxel, epothilones, tamoxifen, 5-fluorouracil, methotrexate, temozolomide, cyclophosphamide, SCH 66336, R115777, L778,123, BMS 214662, gefitinib, erlotinib hydrochloride, antibodies to EGFR, imatinib mesylate, intron, ara-C, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, oxaliplatin, folinic acid, pentostatin, vinblastine, vincristine, vindesine, bleomycin, dactinomycin, daunorubicin, doxorubicin, epirubicin, idarubicin, mithramycin, deoxycoformycin, mitomycin-C, L-asparaginase, teniposide, 17α-ethinylestradiol, diethylstilbestrol, testosterone, prednisone, fluoxymesterone, dromostanolone propionate, testolactone, megestrol acetate, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, carboplatin, hydroxyurea, amsacrine, procarbazine, mitotane, mitoxantrone, levamisole, vinorelbine, anastrazole, letrozole, capecitabine, reloxafine, hexamethylmelamine, bevacizumab, bexxar, velcade, zevalin, trisenox, xeloda, vinorelbine, porfimer, erbitux, liposomal, thiotepa, altretamine, melphalan, trastuzumab, fulvestrant, exemestane, ifosfamide, rituximab, C225, alemtuzumab, clofarabine, cladribine, aphidicolin, sunitinib, dasatinib, tezacitabine, Sml1, triapine, didox, trimidox, amidox, 3-AP, MDL-101,731, bendamustine, ofatumumab, and GS-1101 (also known as CAL-101).

In some embodiments, the chemotherapeutic agent is selected from the group consisting of an alkylating agent (e.g., busulfan, chlorambucil, cisplatin, cyclophosphamide (cytoxan), dacarbazine, ifosfamide, mechlorethamine (mustargen), and melphalan), a nitrosourea (e.g., carmustine, lomustine, semustine, and streptozocin), a triazine (e.g., dacarbazine) an anti-metabolite (e.g., 5-fluorouracil (5-FU), cytarabine (Ara-C), fludarabine, gemcitabine, and methotrexate), a purine analog (e.g., 6-mercaptopurine, 6-thioguanine, and pentostatin (2-deoxycoformycin)), a mitotic inhibitor (e.g., docetaxel, etoposide (VP16), teniposide, paclitaxel, taxol, vinblastine, vincristine, and vinorelbine), an anti-tumor antibiotic (e.g., bleomycin, dactinomycin, daunorubicin, doxorubicin, mitomycin, plicamycin, and idarubicin), a platinum chemotherapeutic agent (e.g., cisplatin and carboplatin), an anthracenedione (e.g., mitoxantrone), a toxin (e.g., ricin A-chain (Burbage, Leukemia research, 21.7 (1997): 681-690), diphtheria toxin A (Massuda et al., Proceedings of the National Academy of Sciences, 94.26 (1997): 14701-14706; Lidor, American journal of obstetrics and gynecology, 177.3 (1997): 579-585), pertussis toxin A subunit, E. coli enterotoxin toxin A subunit, cholera toxin A subunit and Pseudomonas toxin c-terminal), and a gene therapy vector (e.g., a signal transducing protein (e.g., Src, Abl, and Ras), Jun, Fos, and Myc).

In some embodiments, the additional therapeutic agent is an immunotherapeutic agent. An immunotherapeutic agent generally triggers immune effector cells and molecules to target and destroy cells (e.g., cancer cells). The immune effector may be, for example, an antibody specific for a marker on the surface of a cell (e.g. a tumor cell). The antibody alone may serve as an effector of therapy or it may recruit other cells to effect cell killing. Various effector cells include, but are not limited to, cytotoxic T cells and NK cells.

Exemplary immunotherapeutic agents include, but are not limited to, azathioprine, chlorambucil, cyclophosphamide, cyclosporine, daclizumab, infliximab, methotrexate, tacrolimus, immune stimulators (e.g., IL-2, IL-4, IL-12, GM-CSF, tumor necrosis factor; interferons alpha, beta, and gamma; F42K and other cytokine analogs; a chemokine such as MIP-1, MIP-1β, MCP-1, RANTES, IL-8; or a growth factor such FLT3 ligand), an antigenic peptide, polypeptide or protein, or an autologous or allogenic tumor cell composition (see e.g., Ravindranath & Morton, International reviews of immunology, 7.4 (1991): 303-329), hormonal therapy, adrenocorticosteroids, progestins (e.g., hydroxyprogesterone caproate, medroxyprogesterone acetate, and megestrol acetate), estrogens (e.g., diethylstilbestrol and ethinyl estradiol), anti-estrogens (e.g., testosterone propionate and fluoxymesterone), anti-androgens (e.g., flutamide), and gonadotropin-releasing hormone analogs (e.g., leuprolide). Additional immunotherapeutic agents are known in the art, and can be found, for example, in Rosenberg et al, New England Journal of Medicine, 319.25 (1988): 1676-1680; and Rosenberg et al, Annals of surgery, 210.4 (1989): 474).

The therapeutic agents provided herein can be effective over a wide dosage range and are generally administered in an effective amount. It will be understood, however, that the amount of the therapeutic agent actually administered will usually be determined by a physician, according to the relevant circumstances, including the condition to be imaged, the chosen route of administration, the actual compound administered, the age, weight, and response of the individual subject, the severity of the subject's symptoms, and the like.

Additional information can be found in International Application No.: PCT/US2021/036661, filed on Jun. 9, 2021, the relevant disclosures of which are incorporated by reference for the subject matter and purpose referenced herein.

### IV. Kits for Granzyme B Imaging

Also encompassed by the disclosure are kits (e.g., pharmaceutical packs) for Granzyme B imaging, using any of the GZB-binding compounds disclosed herein. The kits provided may comprise a container (e.g., a vial, ampule, bottle, syringe, and/or dispenser package, or other suitable container), in which a pharmaceutical composition as disclosed herein may be placed. In some embodiments, provided kits may optionally further include a second container comprising a pharmaceutical excipient for dilution or suspension of a pharmaceutical composition. In some embodiments, the pharmaceutical composition provided in the first container and the second container are combined to form one unit dosage form. In some embodiments, the kit may comprise additional containers comprising one or more additional therapeutic agents as disclosed herein, for example, anti-inflammatory agents, steroids, immunotherapy agents, chemotherapeutic agents, and therapeutic antibodies as described herein.

In certain embodiments, a kit described herein further includes instructions for using the compounds or composition included in the kit. A kit described herein may also include information as required by a regulatory agency such as the U.S. Food and Drug Administration (FDA). In certain embodiments, the information included in the kits is prescribing information. In certain embodiments, the kits and instructions provide for imaging Granzyme B and for assessing treatment efficacy by any of the therapeutic agents disclosed herein in a subject in need thereof. A kit described herein may include one or more additional pharmaceutical agents described herein as a separate composition.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introuction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1: Radiosynthesis of ¹⁸F-Granzyme B (¹⁸F-GZB)

Typical ¹⁸F-GZB compound RCY ranges from 10-54% using 0.4-1.2 Ci starting activity with synthesis time of 75±10 minutes. Reaction vessel was preloaded with the reaction mixture containing precursor (e.g. 0.2-0.4 mg), AlCl₃·6H₂O (e.g. 34-82 μg), acetic acid/sodium acetate aqueous buffer (e.g. 0.1-0.2 mL, 1 M, pH 3-5), water (e.g. 0.1-0.3 mL) and acetonitrile (e.g. 25-50% of total reaction mixture volume). The [¹⁸F]Fluoride activity was retained on a conditioned anion exchange resin (e.g. Sep-Pak Accell Plus QMA Carbonate Plus Light Cartridge, 46 mg sorbent per cartridge, 40 μm particle size, Waters part No. 186004540) and was then eluted into the reaction vessel using 0.9% saline (e.g. 0.5-0.8 mL). The resulting mixture was heated (e.g. 105° C.) for certain time (e.g. 15 minutes) and then cooled (e.g. 60° C.) prior to dilution with water (e.g. 4-5 mL). The resulting crude was loaded onto a semi-preparative reverse phase HPLC column (e.g. Agilent ZORBAX Eclipse XDB-C18, 5 μm, 9.4×250 mm, Part No. 990967-202) for purification [e.g. mobile phase comprising aqueous acetonitrile (10-20%) solution, pH 1-8]. The HPLC fraction containing the purified ¹⁸F-GZB compound was diluted with 0.5% w/v sodium ascorbate aqueous solution (e.g. 30-50 mL) and was then passed through a conditioned reverse phase cartridge (e.g. Sep-Pak® Light C18 Cartridge, 130 mg sorbent per cartridge, 55-105 μm particle size, Waters part No. WAT0523501). The retained ¹⁸F-GZB was washed with 0.5% w/v sodium ascorbate aqueous solution (e.g. 5-15 mL) and eluted off the cartridge using ethanol (e.g. 1-1.5 mL) into the formulation vial containing 0.5% w/v sodium ascorbate in 0.9% saline (e.g. 6-10 mL). The C18 cartridge was then rinsed with additional 0.5% w/v sodium ascorbate in 0.9% saline (e.g. 3-3.5 mL) and the rinsate was collected into the formulation vial. Certain amount of diluent (10% v/v ethanol and 90% v/v 0.9% saline containing 0.5% w/v sodium ascorbate) can be added to adjust product strength.

To prepare sterile product, the resulting product (¹⁸F-GZB in 10% v/v ethanol and 90% v/v 0.9% saline containing 0.5% w/v sodium ascorbate) was sterile filtered through a 0.22 μm filter (e.g. Millex® GV sterilizing filter, Millipore Part #SLGV033RS; Millex GV 25 mm sterilizing filter, Millipore Part #SLGVV255F; and Millex LG 25 mm sterilizing Filter, Millipore Part #SLLG025SS) into a bulk product vial.

### Example 2: Synthesis of Intermediates

### Preparation of Benzyl ((2R,3S)-2-methoxy-5-oxotetrahydrofuran-3-yl)carbamate and Benzyl ((2S,3S)-2-methoxy-5-oxotetrahydrofuran-3-yl)carbamate

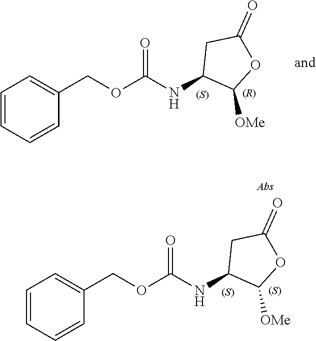

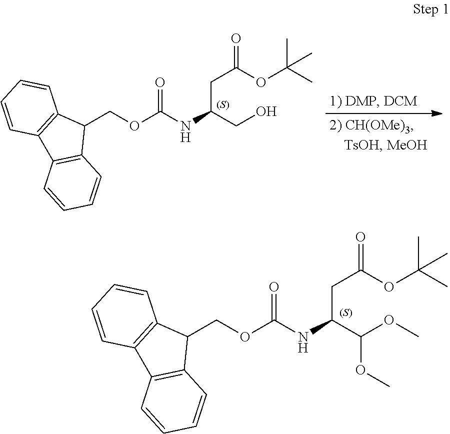

To a solution of tert-butyl (S)-3-((((9H-fluoren-9-yl)methoxy)carbonyl)amino)-4-hydroxybutanoate (11.5 g, 28.9 mmol) in dichloromethane (120 mL) was added Dess-Martin periodinane (18.6 g, 43.4 mmol). The mixture was stirred at 20° C. for 3 h under N₂. The reaction mixture was quenched with saturated sodium bicarbonate solution (200 mL) and extracted with dichloromethane (200 mL×3). The combined organic layers were washed with brine (300 mL), dried over sodium sulfate, filtered, and concentrated under reduced pressure. The residue was combined with another reaction of the same scale and purified by flash silica gel chromatography (ISCO; 220 g, 0-40% ethyl acetate/petroleum ether 100 m/min). tert-butyl (S)-3-((((9H-fluoren-9-yl)methoxy)carbonyl)amino)-4-oxobutanoate (18 g, 45.52 mmol) was obtained as yellow oil and used for the next step.

### Step 2:

To a solution of tert-butyl (S)-3-((((9H-fluoren-9-yl)methoxy)carbonyl)amino)-4-oxobutanoate (12 g, 30.35 mmol) in methanol (60 mL) was added trimethoxymethane (13.7 g, 129 mmol) and p-toluenesulfonic acid (227 mg, 1.3 mmol,). The reaction mixture was stirred at 20° C. for 16 h. The reaction mixture was concentrated under reduced pressure to remove solvent to give the crude product. The crude product was combined with another reaction starting from 6 g of tert-butyl (S)-3-((((9H-fluoren-9-yl)methoxy)carbonyl)amino)-4-oxobutanoate. The combined mixture was dissolved in dichloromethane (300 mL). The dichloromethane solution was washed with sat. NaHCO₃ (200 mL) and brine (200 mL), dried over Na₂SO₄, filtered and concentrated. The residue was purified by flash silica gel chromatography (ISCO; 220 g, 0-25% ethyl acetate/Petroleum ether, 100 m/min) to afford tert-butyl (S)-3-((((9H-fluoren-9-yl)methoxy)carbonyl)amino)-4,4-dimethoxybutanoate (13.6 g, 29.9 mmol) as a colorless oil.

ES/MS m/z 464.2 (M+Na)⁺.

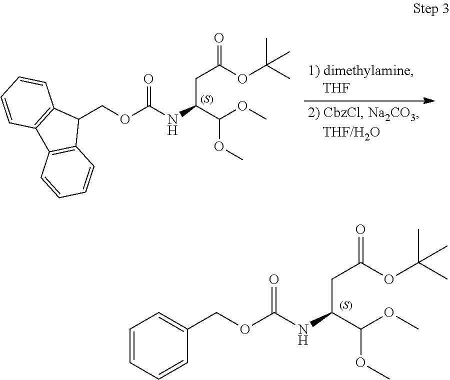

To a solution of tert-butyl (S)-3-((((9H-fluoren-9-yl)methoxy)carbonyl)amino)-4,4-dimethoxybutanoate (13.6 g, 29.9 mmol) in tetrahydrofuran (80 mL) was added diethylamine (80 mL, 760 mmol). The reaction mixture was stirred at 20° C. for 5 h. The reaction mixture was concentrated under reduced pressure to remove solvent to give the crude tert-butyl (S)-3-amino-4,4-dimethoxybutanoate (14 g) as a colorless oil used for the next step without further purification.

### Step 4:

To a solution of tert-butyl (S)-3-amino-4,4-dimethoxybutanoate (14 g) and sodium carbonate (7.2 g, 68 mmol) in tetrahydrofuran (80 mL) and water (4 mL) was added a solution of benzyl chloroformate (8.6 g, 50 mmol) in tetrahydrofuran (20 mL) slowly at 0° C. The reaction mixture was stirred at room temperature until TLC showed the reaction was completed. Water (80 mL) was added, and the mixture was extracted with ethyl acetate (100 mL×3). The combined organic layer was washed with brine (150 mL), dried over sodium sulfate, filtered and concentrated. The residue was purified by flash silica gel chromatography (ISCO; 220 g silica gel flash column, 0-40% ethyl acetate/petroleum ether, 100 mL/min) to afford tert-butyl (S)-3-(((benzyloxy)carbonyl)amino)-4,4-dimethoxybutanoate (11.7 g, 32.82 mmol) as a light-yellow oil.

¹H NMR (400 MHz, CDCl3) δ: 7.39-7.29 (m, 5H), 5.33 (d, J=8.8 Hz, 1H), 5.17-5.05 (m, 2H), 4.35 (d, J=3.6 Hz, 1H), 4.25-4.16 (m, 1H), 3.45-3.38 (s, 6H), 2.57-2.41 (m, 2H), 1.43 (s, 9H).

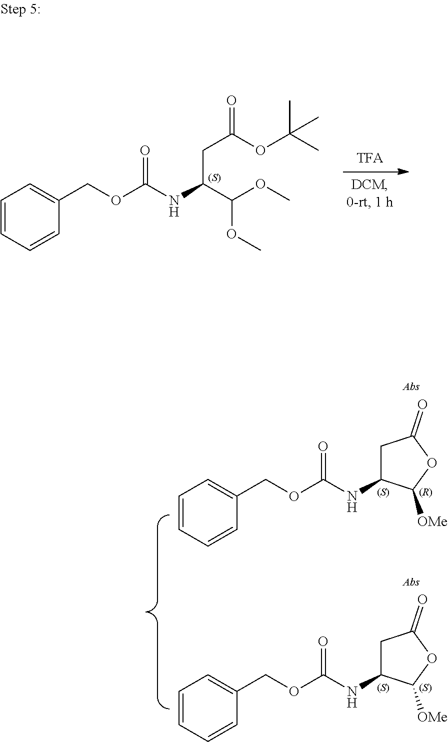

To a solution of tert-butyl (S)-3-(((benzyloxy)carbonyl)amino)-4,4-dimethoxybutanoate (10.2 g, 27.4 mmol) in dichloromethane (100 mL) was added TFA (10 mL, 129 mmol) and anisole (1.0 mL, 9.1 mmol) at 0° C. The reaction mixture was warmed to 20° C. and stirred for 1 h. The reaction mixture was concentrated under reduced pressure to remove solvent. The residue was combined with another reaction starting from 1.5 g tert-butyl (S)-3-(((benzyloxy)carbonyl)amino)-4,4-dimethoxybutanoate. The residue was purified several times by flash silica gel chromatography (ISCO, 220 g, 0-1% MeOH/DCM, 80 mL/min) to afford two compounds:

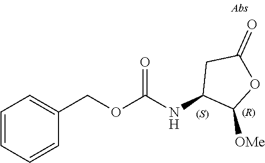

Benzyl ((2R,3S)-2-methoxy-5-oxotetrahydrofuran-3-yl)carbamate (3.85 g, 12.2 mmol) was obtained as white solid.

1H NMR (400 MHz, DMSO): δ 7.58 (d, J=7.9 Hz, 1H), 7.43-7.29 (m, 5H), 5.43 (d, J=5.0 Hz, 1H), 5.07 (s, 2H), 4.47-4.39 (m, 1H), 3.42 (s, 3H), 2.71 (dd, J=17.2, 8.9 Hz, 1H), 2.52 (dd, J=17.2, 9.9 Hz, 1H).

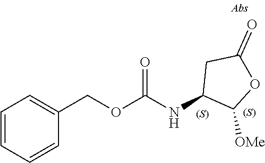

Benzyl ((2S,3S)-2-methoxy-5-oxotetrahydrofuran-3-yl)carbamate (1.93 g, 6.40 mmol) was obtained as colorless oil.

¹H NMR (400 MHz, DMSO): δ 7.88 (s, 1H), 7.43-7.29 (m, 5H), 5.29 (d, J=1.5 Hz, 1H), 5.07 (s, 2H), 4.06-4.00 (m, 1H), 3.42 (s, 3H), 2.98 (dd, J=18.1, 8.3 Hz, 1H), 2.41 (dd, J=18.1, 2.9 Hz, 1H).

### Preparation of Benzyl ((2S,3R)-2-methoxy-5-oxotetrahydrofuran-3-yl)carbamate and Benzyl ((2R,3R)-2-methoxy-5-oxotetrahydrofuran-3-yl)carbamate

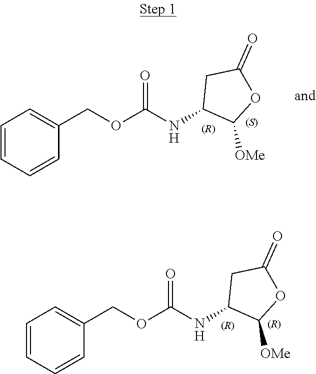

Intermediate tert-butyl (R)-3-(((benzyloxy)carbonyl)amino)-4,4-dimethoxybutanoate was prepared following similar synthetic procedure above.

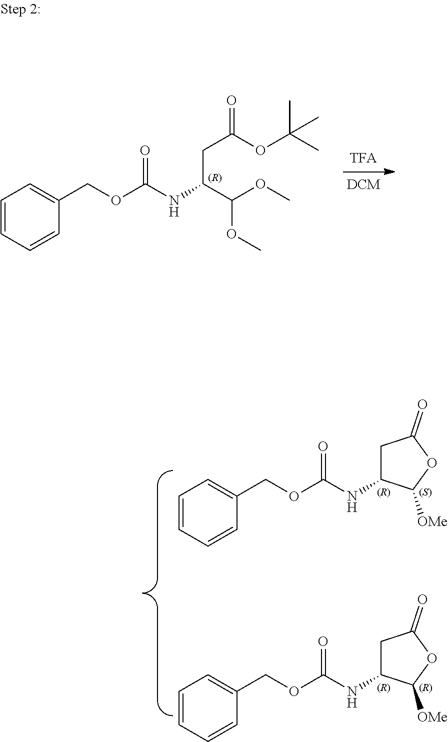

To a solution of tert-butyl (R)-3-(((benzyloxy)carbonyl)amino)-4,4-dimethoxybutanoate (4 g, 10.87 mmol) in dichloromethane (40 mL) was added TFA (4 mL, 51.4 mmol) and Anisole (0.4 mL, 4 mmol). The reaction mixture was stirred at 20° C. for 1 h. The reaction mixture was concentrated under reduced pressure to remove solvent. The residue was combined with two other batches reactions of starting from 5 g of tert-butyl (R)-3-(((benzyloxy)carbonyl)amino)-4,4-dimethoxybutanoate each. It was purified by flash silica gel chromatography (ISCO; 220 g, 0-1% MeOH/dichloromethane,100 mL/min).

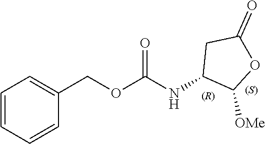

Benzyl ((2S,3R)-2-methoxy-5-oxotetrahydrofuran-3-yl)carbamate (2.2 g, 8.0 mmol) was obtained as white solid.

1H NMR (400 MHz, CDCl₃) δ: 7.41-7.33 (m, 5H), 5.38-5.29 (m, 2H), 5.15-5.12 (s, 2H), 4.64-4.52 (m, 1H), 3.54 (s, 3H), 2.86 (dd, J=8.4, 17.3 Hz, 1H), 2.45 (dd, J=10.4, 17.3 Hz, 1H).

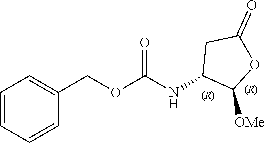

Benzyl ((2R,3R)-2-methoxy-5-oxotetrahydrofuran-3-yl)carbamate (1.5 g, 5.3 mmol) was obtained as colorless oil.

1H NMR (400 MHz, CDCl₃) δ: 7.42-7.31 (m, 5H), 5.31 (s, 1H), 5.17-5.10 (s, 2H), 5.06-4.94 (m, 1H), 4.22 (t, J=6.6 Hz, 1H), 3.51 (s, 3H), 3.00 (dd, J=7.8, 18.0 Hz, 1H), 2.40 (d, J=18.0 Hz, 1H).

### Preparation of (R)-2-(2-(4-(((9H-fluoren-9-yl)methoxy)carbonyl)piperazin-1-yl)acetamido)-5-(benzyloxy)-5-oxopentanoic acid

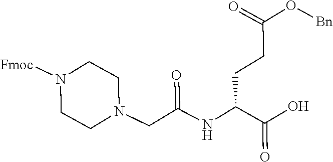

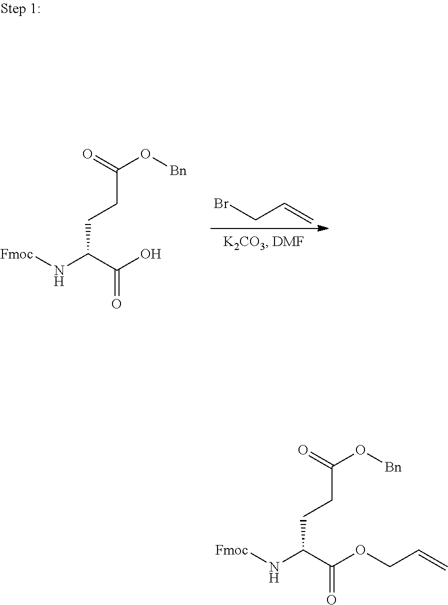

To a solution of (R)-2-((((9H-fluoren-9-yl)methoxy)carbonyl)amino)-5-(benzyloxy)-5-oxopentanoic acid (3.3 g, 7.2 mmol) in dimethylformamide (30 mL) was added potassium carbonate (1.82 g, 13.0 mmol) and allyl bromide (1.58 g, 13.1 mmol). The reaction mixture was stirred at 20° C. for 12 h. Water (80 mL) was added. The mixture was extracted with ethyl acetate (100 mL). The combined organic layer was washed with brine (3×60 mL), dried over Sodium sulfate, and concentrated in vacuo to give the crude 1-allyl 5-benzyl (((9H-fluoren-9-yl)methoxy)carbonyl)-D-glutamate (4.1 g, 6.6 mmol) as yellow oil.

ES/MS m/z 500.1 (M+H)⁺.

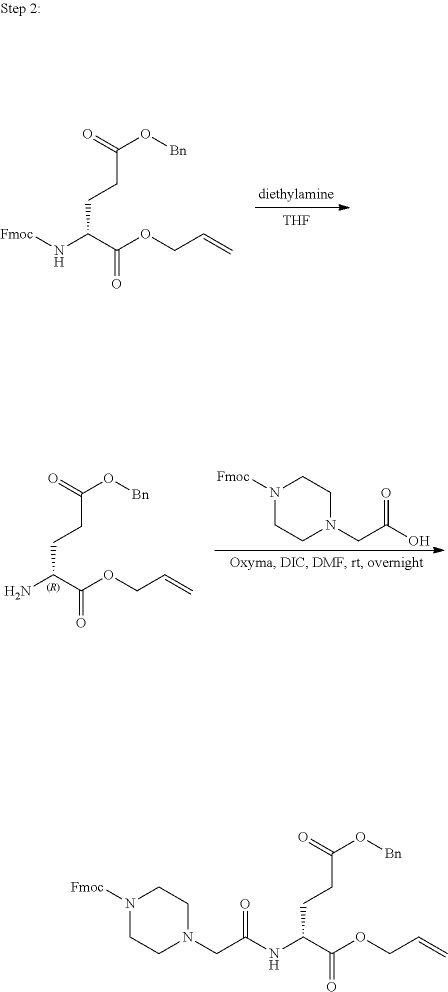

To a solution of 1-allyl 5-benzyl (((9H-fluoren-9-yl)methoxy)carbonyl)-D-glutamate (4.1 g, 6.6 mmol) in tetrahydrofuran (30 mL) was added diethylamine (1.82 g, 24.8 mmol). The reaction mixture was stirred at 20° C. for 3 h. The solvent was removed under reduced pressure to give the crude 1-allyl 5-benzyl D-glutamate (1.9 g) as a yellow oil, which was used for the next directly. To a solution of 2-(4-(((9H-fluoren-9-yl)methoxy)carbonyl)piperazin-1-yl)acetic acid (2.5 g, 6.8 mmol) and ethyl cyanohydroxyiminoacetate (1.2 g, 8.2 mmol) in dimethylformamide (20 mL) was added N,N-diisopropylcarbodiimide (1.1 g, 8.5 mmol) at 0° C. The reaction mixture was stirred at 0° C. for 0.5 h under N₂. Then crude 1-allyl 5-benzyl D-glutamate (1.9 g) in dimethylformamide (5 mL) was added. The reaction mixture was stirred at 20° C. for 16 h under N₂. H₂O (50 mL) was added, and the reaction mixture was extracted with ethyl acetate (8×80 mL). The combined organic layers were washed with brine (3×100 mL), dried over Na₂SO₄, filtered, and concentrated under reduced pressure. The residue was purified by flash silica gel chromatography (ISCO, 40 g, 0-5% MeOH/DCM with 0.3% NH₃·H₂O as additive). 1-Allyl 5-benzyl (2-(4-(((9H-fluoren-9-yl)methoxy)carbonyl)piperazin-1-yl)acetyl)-D-glutamate (2.5 g, 2.9 mmol) was obtained as a yellow oil.

ES/MS m/z 626.3 (M+H)⁺.

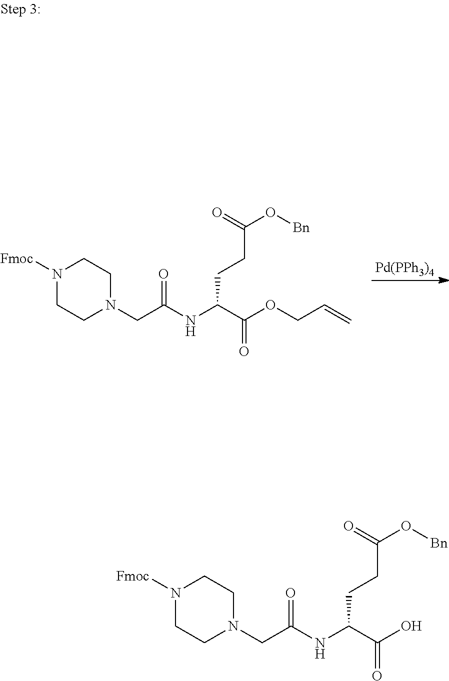

To a solution of 1-allyl 5-benzyl (2-(4-(((9H-fluoren-9-yl)methoxy)carbonyl)piperazin-1-yl)acetyl)-D-glutamate (2.5 g, 2.9 mmol) and morpholine (700 mg, 7.9 mmol) in tetrahydrofuran (40 mL) was added tetrakis(triphenylphosphine)palladium(0) (140 mg, 0.12 mmol). The mixture was stirred at room temperature for 12 h. The solution was removed under reduced pressure to give the crude product. The crude product was purified by C18 prep-HPLC (0.225% formic acid in H₂O—CH₃CN). The fractions were combined and lyophilized to afford (R)-2-(2-(4-(((9H-fluoren-9-yl)methoxy)carbonyl)piperazin-1-yl)acetamido)-5-(benzyloxy)-5-oxopentanoic acid (510 mg, 0.84 mmol) as brown oil.

ES/MS m/z 586.6 (M+H)⁺.

### Preparation of 2-(4,7-bis(2-(benzyloxy)-2-oxoethyl)-1,4,7-triazonan-1-yl)acetic acid

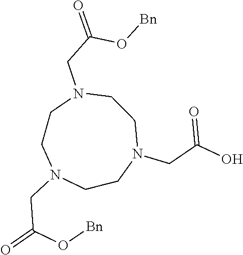 [SMILES: O=C(O)CN1CCN(CC(=O)OCc2ccccc2)CCN(CC(=O)OCc2ccccc2)CC1]

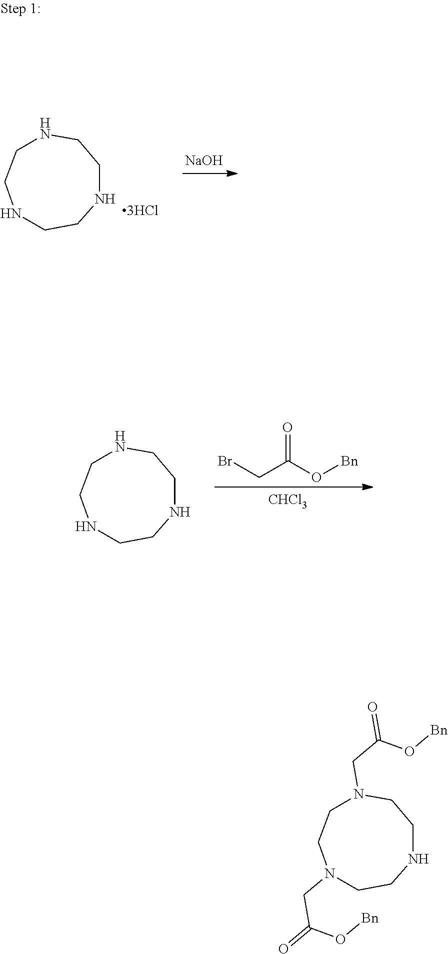

To a solution of 1,4,7-triazonane hydrochloric acid salt (15 g, 61.0 mmol) in toluene (180 mL) was added sodium hydroxide (7.4 g, 180 mmol) in water (12 mL). The reaction mixture was stirred at 135° C. for 12 h with a Dean-Stark condenser. Additional sodium hydroxide (3.9 g, 97 mmol) was added, and the mixture was stirred at 130° C. for 2 h. The mixture was filtered while hot and the cake was washed with ethyl acetate (3×15 mL). The filtrates were concentrated to give 1,4,7-triazonane (7.0 g, 54 mmol) as a white solid.

To a solution of 1,4,7-triazonane (4 g, 31.0 mmol) in chloroform (120 mL) was added benzyl 2-bromoacetate (13.5 g, 58.9 mmol) in chloroform (120 mL) drop-wise for 1.5 hour at −10° C. The reaction mixture was warmed to room temperature and stirred for 12 h. The reaction mixture was concentrated under reduced pressure to remove solvent. The residue was purified by flash silica gel chromatography (ISCO, 220 g, 0-8% MeOH(2% 7M NH₃ in methanol)/DCM gradient). Dibenzyl 2,2′-(1,4,7-triazonane-1,4-diyl)diacetate (4.6 g, 10 mmol) was obtained as colorless oil.

ES/MS m/z 426.1 (M+H)⁺.

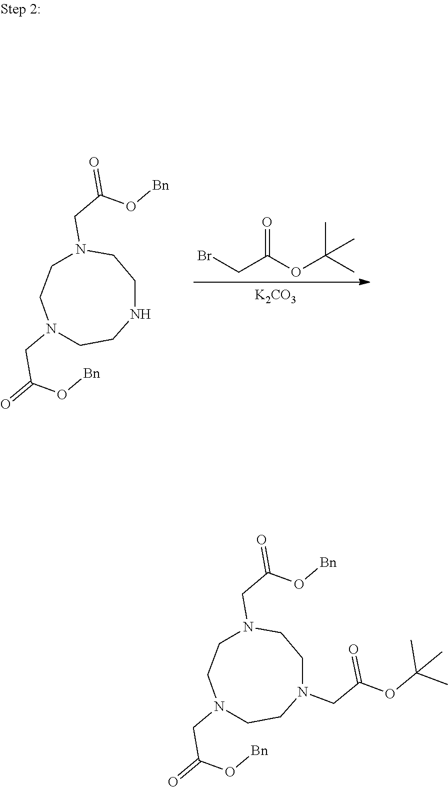

To a solution of dibenzyl 2,2′-(1,4,7-triazonane-1,4-diyl)diacetate (6.1 g, 13 mmol) in acetonitrile (150 mL) was added potassium carbonate (2.97 g, 21.3 mmol) and tert-butyl 2-bromoacetate (2.66 g, 13.6 mmol). The reaction mixture was stirred at room temperature for 12 h. The reaction mixture was concentrated under reduced pressure to remove solvent. The crude product was purified by C18 prep-HPLC (0.225% formic acid in H₂O/CH₃CN). The fractions were combined and lyophilized to give dibenzyl 2,2′-(7-(2-(tert-butoxy)-2-oxoethyl)-1,4,7-triazonane-1,4-diyl)diacetate (5.7 g, 9.4 mmol) as colorless oil.

ES/MS m/z 540.2 (M+H)⁺.

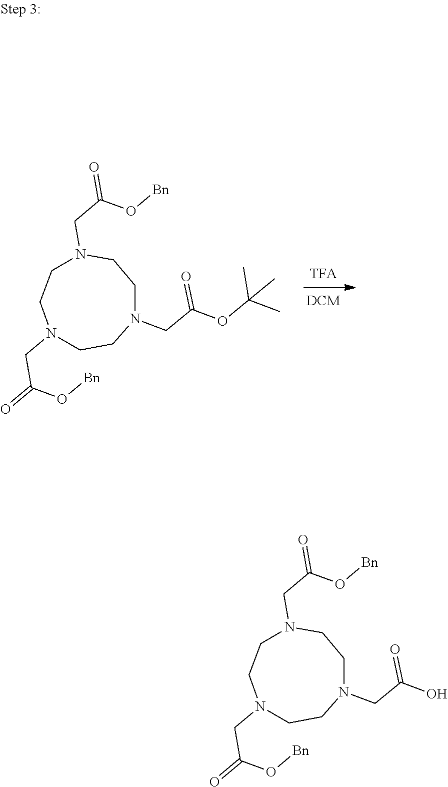

To a solution of dibenzyl 2,2′-(7-(2-(tert-butoxy)-2-oxoethyl)-1,4,7-triazonane-1,4-diyl)diacetate (5.7 g, 11 mmol) in dichloromethane (30 mL) was added trifluoracetic acid (30 mL). The reaction mixture was stirred at room temperature for 20 h. The reaction mixture was concentrated under reduced pressure to remove solvent. Sat. NaHCO₃ was added to adjust the pH of the mixture to 8. The mixture was then lyophilized to give the crude 2-(4,7-bis(2-(benzyloxy)-2-oxoethyl)-1,4,7-triazonan-1-yl)acetic acid (4.7 g, 9.7 mmol) as yellow oil. The crude product was used for the next step without further purification.

ES/MS m/z 540.2 (M+H)⁺.

### Preparation of Dibenzyl 2,2′-(7-((1-(2-((2,5-dioxopyrrolidin-1-yl)oxy)-2-oxoethyl)piperidin-4-yl)methyl)-1,4,7-triazonane-1,4-diyl)diacetate

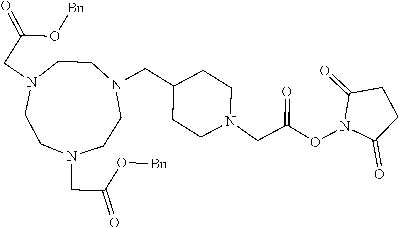 [SMILES: O=C(CN1CCN(CC(=O)OCc2ccccc2)CCN(CC2CCN(CC(=O)ON3C(=O)CCC3=O)CC2)CC1)OCc1ccccc1]

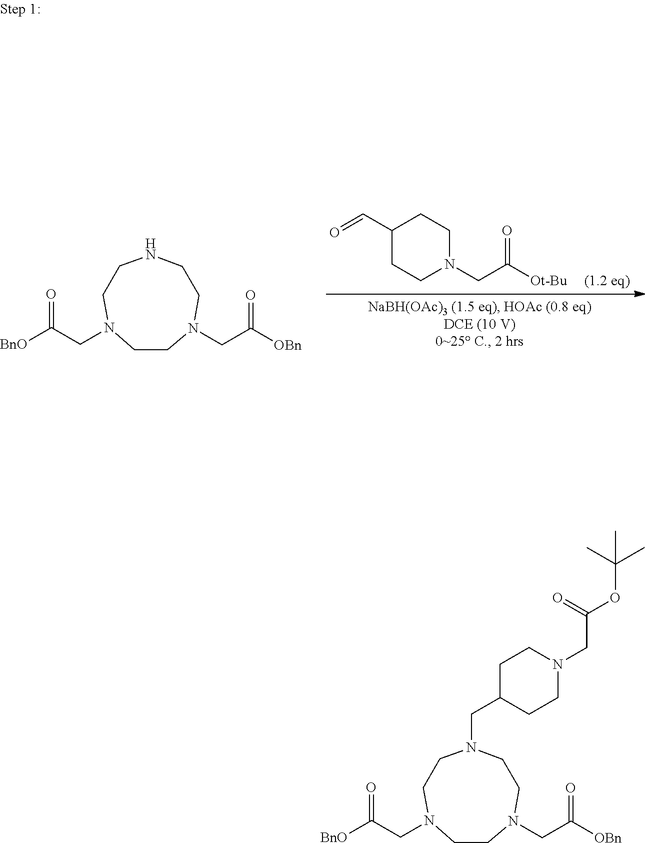

To a solution of dibenzyl 2,2′-(1,4,7-triazonane-1,4-diyl)diacetate (28.0 g, 65.8 mmol) and tert-butyl 2-(4-formylpiperidin-1-yl)acetate (17.9 g, 79.0 mmol) in DCE (245 mL) was added AcOH (3.16 g, 52.6 mmol, 3.01 mL, 0.80 eq) at 0° C. and the mixture was stirred at 0° C. for 1 hr. NaBH(OAc)₃ (20.9 g, 98.7 mmol, 1.50 eq) was then added and the resulting mixture was stirred at 25° C. for 2 hrs. The reaction mixture was quenched by addition NaHCO₃ (500 mL), and then extracted with EA (500 mL×2). The combined organic layers were dried over Na₂SO₄, filtered, and concentrated under reduced pressure to give dibenzyl 2,2′-(7-((1-(2-(tert-butoxy)-2-oxoethyl)piperidin-4-yl)methyl)-1,4,7-triazonane-1,4-diyl)diacetate (50.0 g, crude) as a brown oil. The crude product was used for next step directly without further purification.

ES/MS m/z 637.4 (M+H)⁺.

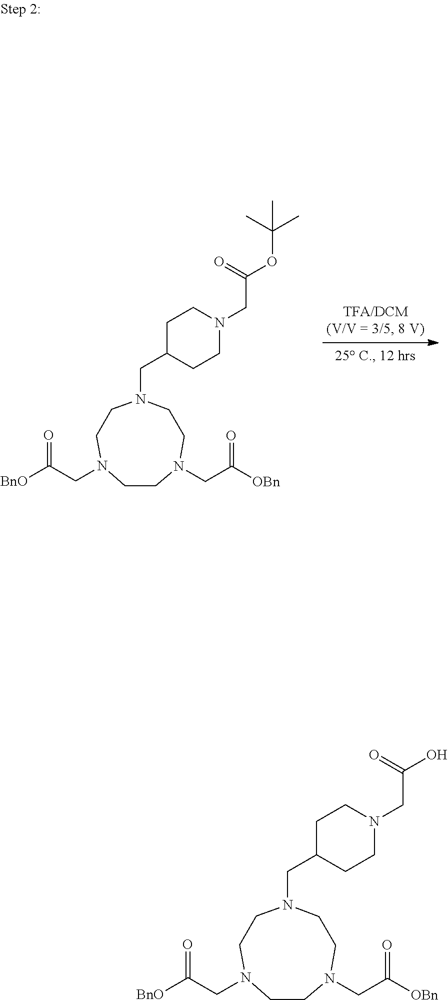

To a solution of dibenzyl 2,2′-(7-((1-(2-(tert-butoxy)-2-oxoethyl)piperidin-4-yl)methyl)-1,4,7-triazonane-1,4-diyl)diacetate (50.0 g, 78.5 mmol) in DCM (350 mL) was added TFA (231 g, 2.03 mol, 150 mL). The mixture was stirred at 20° C. for 12 hrs. The reaction mixture was concentrated under reduced pressure to remove solvent. The residue was purified by prep-HPLC (TFA condition) and the TFA salt was dissolved in deionized water and dissociated with chloride ion resin to form the HCl salt and 2-(4-((4,7-bis(2-(benzyloxy)-2-oxoethyl)-1,4,7-triazonan-1-yl)methyl)piperidin-1-yl)acetic acid, HCl salt (20.0 g, 31.0 mmol) was obtained as a light brown foam. ES/MS m/z 581.2 (M+H)⁺.

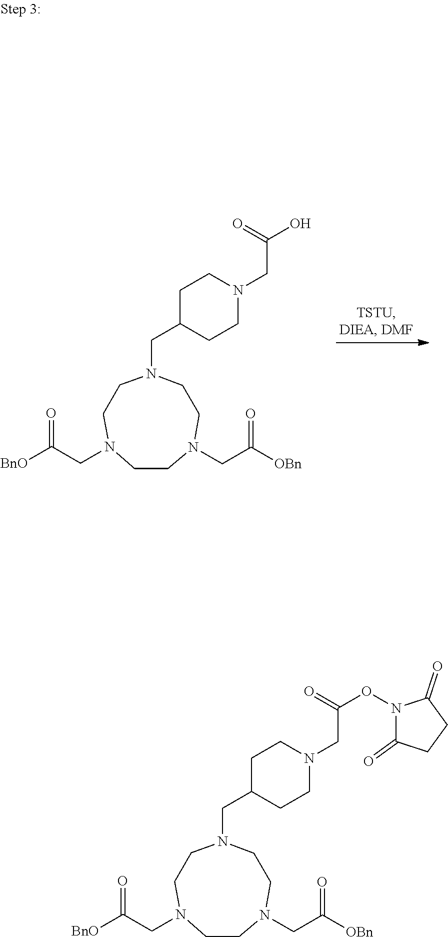 [SMILES: O=C(O)CN1CCC(CN2CCN(CC(=O)OCc3ccccc3)CCN(CC(=O)OCc3ccccc3)CC2)CC1 ; "TSTU,DIEA, DMF" ; O=C(CN1CCN(CC(=O)OCc2ccccc2)CCN(CC2CCN(CC(=O)ON3C(=O)CCC3=O)CC2)CC1)OCc1ccccc1]

To a solution of 2-(4-((4,7-bis(2-(benzyloxy)-2-oxoethyl)-1,4,7-triazonan-1-yl)methyl)piperidin-1-yl)acetic acid (0.4 g, 2.4 mmol) and N,N-diisopropylethylamine (470 mg, 3.6 mmol) in DMF (20 mL) was added TSTU (tetramethyl-O—(N-succinimidyl)uronium tetrafluoroborate, 820 mg, 2.6 mmol) at 0° C. under N₂. Then the mixture was stirred at 20° C. for 1 h. The reaction mixture of dibenzyl 2,2′-(7-((1-(2-((2,5-dioxopyrrolidin-1-yl)oxy)-2-oxoethyl)piperidin-4-yl)methyl)-1,4,7-triazonane-1,4-diyl)diacetate (2.4 mmol) was used for the next step directly without further purification.

### Example 3: Synthesis of Peptide Acid Intermediate

General peptide synthesis procedure A: Peptides were synthesized following standard Fmoc solid-phase peptide synthesis procedures using 2-CTC (2-Chlorotrityl chloride) resin. Final peptides were deprotected and cleaved from the resin via 20% 1,1,1,3,3,3-Hexafluoro-2-propanol in Dichloromethane treatment: The resin was treated with 20% 1,1,1,3,3,3-Hexafluoro-2-propanol in Dichloromethane (15-20 ml of solution per gram of resin) and shook for 10 min. The solution was drained into round bottom flask. The process was repeated twice with fresh aliquots of 20% 1,1,1,3,3,3-Hexafluoro-2-propanol in Dichloromethane (15-20 ml of solution per gram of resin) and shook for 30 min each. The solutions from all treatments were drained and combined. Crude peptides were concentrated, then subjected to preparative HPLC or reverse phase C18 column purification (water with or without appropriate modifier/acetonitrile mobile phase). Product-containing fractions were collected and lyophilized to afford peptides as solids.

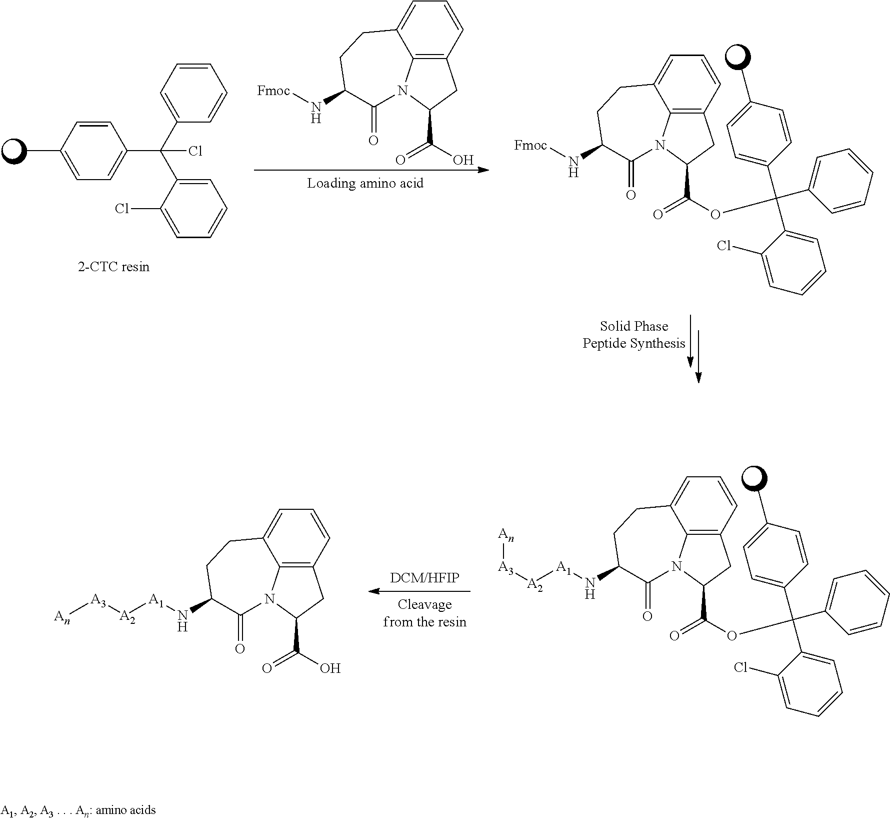

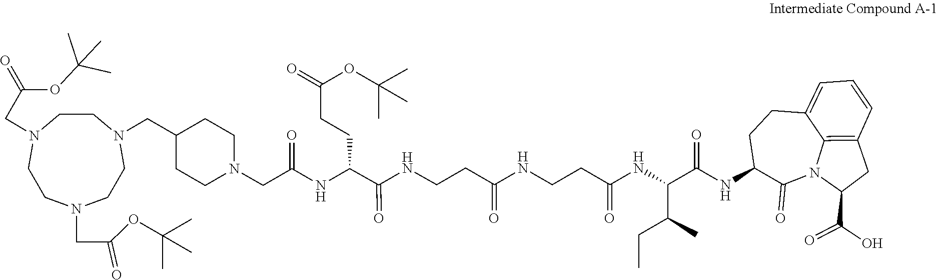 [SMILES: CC[C@H](C)[C@H](NC(=O)CCNC(=O)CCNC(=O)[C@@H](CCC(=O)OC(C)(C)C)NC(=O)CN1CCC(CN2CCN(CC(=O)OC(C)(C)C)CCN(CC(=O)OC(C)(C)C)CC2)CC1)C(=O)N[C@H]1CCc2cccc3c2N(C1=O)[C@H](C(=O)O)C3]

Following the general procedure A, (3S,6S)-3-((2S,13R)-13-(2-(4-((4,7-bis(2-(tert-butoxy)-2-oxoethyl)-1,4,7-triazonan-1-yl)methyl)piperidin-1-yl)acetamido)-2-((S)-sec-butyl)-18,18-dimethyl-4,8,12,16-tetraoxo-17-oxa-3,7,11-triazanonadecanamido)-4-oxo-1,2,3,4,6,7-hexahydroazepino[3,2,1-hi]indole-6-carboxylic acid (1005.2 mg) was synthesized using solid phase, and was isolated as a gray powder.

ES/MS m/z 1181.8 (M+H)⁺.

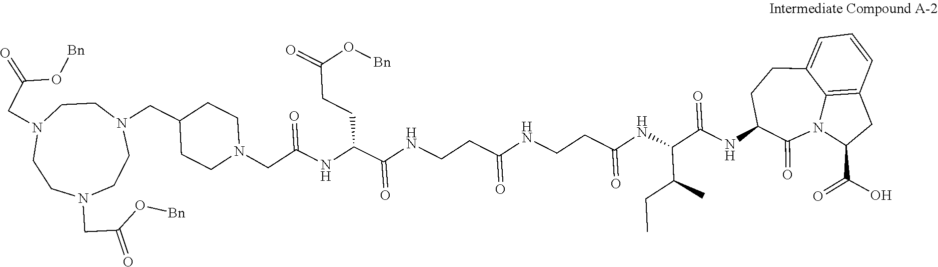 [SMILES: CC[C@H](C)[C@H](NC(=O)CCNC(=O)CCNC(=O)[C@@H](CCC(=O)OCc1ccccc1)NC(=O)CN1CCC(CN2CCN(CC(=O)OCc3ccccc3)CCN(CC(=O)OCc3ccccc3)CC2)CC1)C(=O)N[C@H]1CCc2cccc3c2N(C1=O)[C@H](C(=O)O)C3]

Following procedure A, (3S,6S)-3-((6R,17S)-6-(2-(4-((4,7-bis(2-(benzyloxy)-2-oxoethyl)-1,4,7-triazonan-1-yl)methyl)piperidin-1-yl)acetamido)-17-((S)-sec-butyl)-3,7,11,15-tetraoxo-1-phenyl-2-oxa-8,12,16-triazaoctadecan-18-amido)-4-oxo-1,2,3,4,6,7-hexahydroazepino[3,2,1-hi]indole-6-carboxylic acid (354 mg) was synthesized using solid phase as a white solid.

ES/MS m/z 1284.1 (M+H)⁺.

For this specific example, modifications were made to the solid phase synthesis protocol:

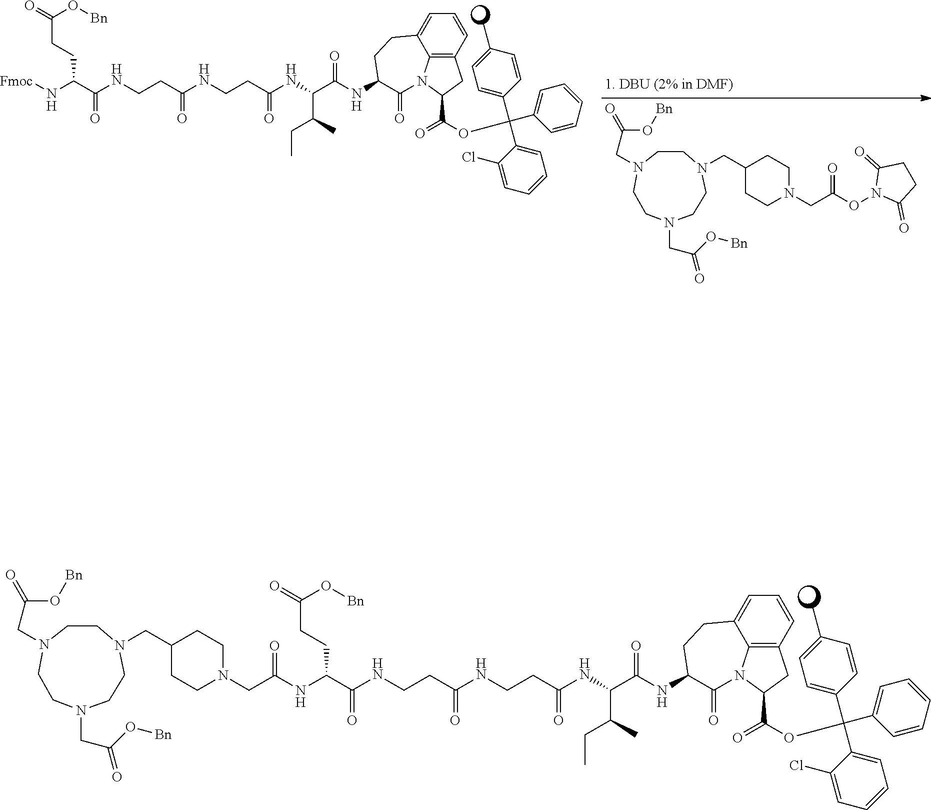

To the fritted glass funnel containing (3S,6S)-3-((5R,16S)-5-(3-(benzyloxy)-3-oxopropyl)-16-((S)-sec-butyl)-1-(9H-fluoren-9-yl)-3,6,10,14-tetraoxo-2-oxa-4,7,11,15-tetraazaheptadecan-17-amido)-4-oxo-1,2,3,4,6,7-hexahydroazepino[3,2,1-hi]indole-6-carboxylic acid on 2-CTC resin (2 mmol) was added DBU (25 mL, 2% in DMF) and stirred for 2 min. The mixture was filtered and DBU (25 mL, 2% in DMF) was added and stirred for 2 min. The solution was then removed from the resin, and the resin was washed with DMF (10 mL×8).

A mixture of the resulting resin and crude dibenzyl 2,2′-(7-((1-(2-((2,5-dioxopyrrolidin-1-yl)oxy)-2-oxoethyl)piperidin-4-yl)methyl)-1,4,7-triazonane-1,4-diyl)diacetate (2.4 mmol) in DMF (20 mL) in the fritted glass funnel was shook at room temperature for 12 h. The solution was then removed from the resin, and the resin was washed with the DMF (30 mL*2), then DCM (30 mL*2), then DMF (30 mL*2) and then finally DCM (30 mL*2).

The resin cleavage was done following general procedure A.

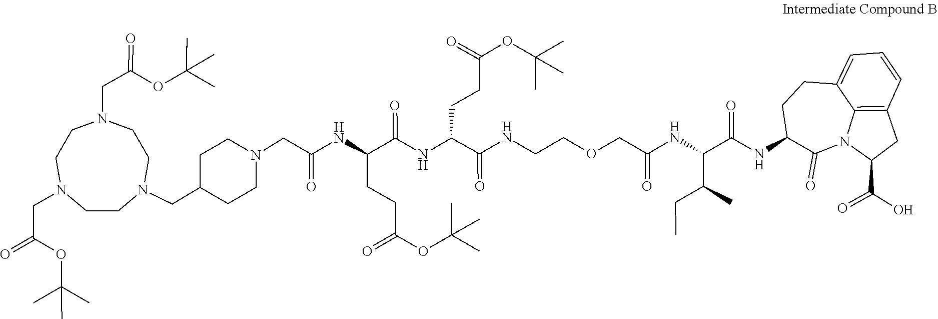 [SMILES: CC[C@H](C)[C@H](NC(=O)COCCNC(=O)[C@@H](CCC(=O)OC(C)(C)C)NC(=O)[C@@H](CCC(=O)OC(C)(C)C)NC(=O)CN1CCC(CN2CCN(CC(=O)OC(C)(C)C)CCN(CC(=O)OC(C)(C)C)CC2)CC1)C(=O)N[C@H]1CCc2cccc3c2N(C1=O)[C@H](C(=O)O)C3]

Following the general procedure A, (3S,6S)-3-((2S,11R,14R)-14-(2-(4-((4,7-bis(2-(tert-butoxy)-2-oxoethyl)-1,4,7-triazonan-1-yl)methyl)piperidin-1-yl)acetamido)-11-(3-(tert-butoxy)-3-oxopropyl)-2-((S)-sec-butyl)-19,19-dimethyl-4,10,13,17-tetraoxo-6,18-dioxa-3,9,12-triazaicosanamido)-4-oxo-1,2,3,4,6,7-hexahydroazepino[3,2,1-hi]indole-6-carboxylic acid (555.8 mg) was synthesized using solid phase, and was isolated as an off-white powder.

ES/MS m/z 1325.6 (M+H)⁺.

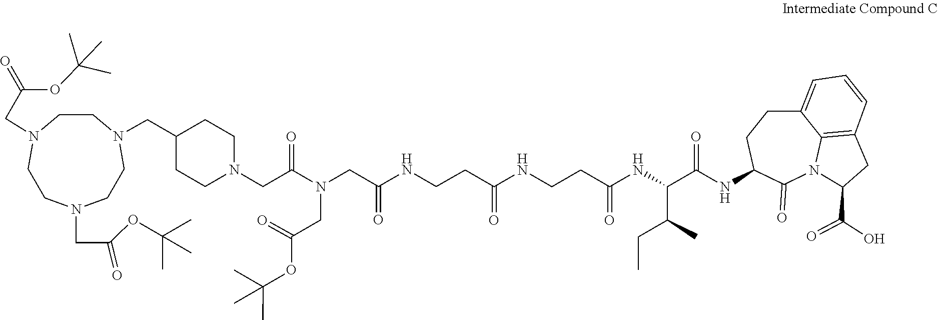 [SMILES: CC[C@H](C)[C@H](NC(=O)CCNC(=O)CCNC(=O)CN(CC(=O)OC(C)(C)C)C(=O)CN1CCC(CN2CCN(CC(=O)OC(C)(C)C)CCN(CC(=O)OC(C)(C)C)CC2)CC1)C(=O)N[C@H]1CCc2cccc3c2N(C1=O)[C@H](C(=O)O)C3]

Following the general procedure A, (3S,6S)-3-((S)-6-(2-(4-((4,7-bis(2-(tert-butoxy)-2-oxoethyl)-1,4,7-triazonan-1-yl)methyl)piperidin-1-yl)acetyl)-18-((S)-sec-butyl)-2,2-dimethyl-4,8,12,16-tetraoxo-3-oxa-6,9,13,17-tetraazanonadecan-19-amido)-4-oxo-1,2,3,4,6,7-hexahydroazepino[3,2,1-hi]indole-6-carboxylic acid (340.9 mg) was synthesized using solid phase, and was isolated as an off-white powder.

ES/MS m/z 1167.8 (M+H)⁺.

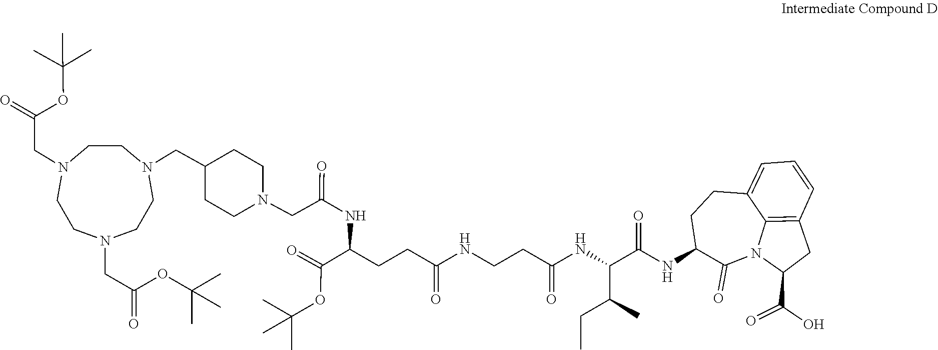 [SMILES: CC[C@H](C)[C@H](NC(=O)CCNC(=O)CC[C@H](NC(=O)CN1CCC(CN2CCN(CC(=O)OC(C)(C)C)CCN(CC(=O)OC(C)(C)C)CC2)CC1)C(=O)OC(C)(C)C)C(=O)N[C@H]1CCc2cccc3c2N(C1=O)[C@H](C(=O)O)C3]

Following procedure A, (3S,6S)-3-((2S,3S)-2-(3-((S)-4-(2-(4-((4,7-bis(2-(tert-butoxy)-2-oxoethyl)-1,4,7-triazonan-1-yl)methyl)piperidin-1-yl)acetamido)-5-(tert-butoxy)-5-oxopentanamido)propanamido)-3-methylpentanamido)-4-oxo-1,2,3,4,6,7-hexahydroazepino[3,2,1-hi]indole-6-carboxylic acid (325.0 mg) was synthesized using solid phase, and was isolated as an off-white powder.

ES/MS m/z 1110.6 (M+H)⁺.

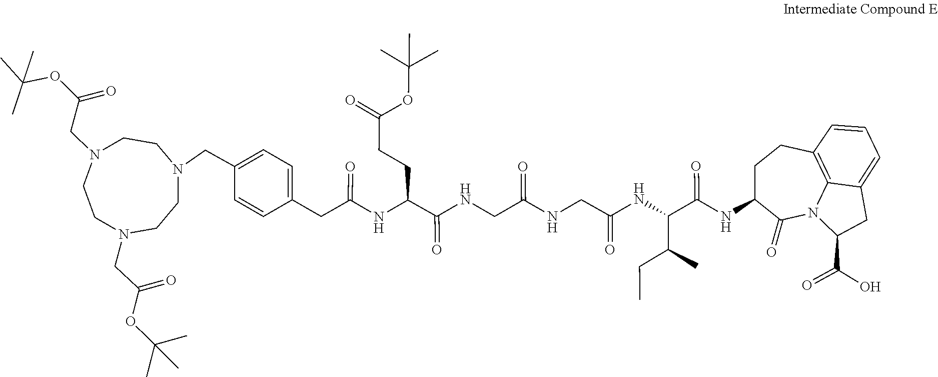 [SMILES: CC[C@H](C)[C@H](NC(=O)CNC(=O)CNC(=O)[C@H](CCC(=O)OC(C)(C)C)NC(=O)Cc1ccc(CN2CCN(CC(=O)OC(C)(C)C)CCN(CC(=O)OC(C)(C)C)CC2)cc1)C(=O)N[C@H]1CCc2cccc3c2N(C1=O)[C@H](C(=O)O)C3]

Following procedure A, (3S,6S)-3-((2S,11S)-11-(2-(4-((4,7-bis(2-(tert-butoxy)-2-oxoethyl)-1,4,7-triazonan-1-yl)methyl)phenyl)acetamido)-2-((S)-sec-butyl)-16,16-dimethyl-4,7,10,14-tetraoxo-15-oxa-3,6,9-triazaheptadecanamido)-4-oxo-1,2,3,4,6,7-hexahydroazepino[3,2,1-hi]indole-6-carboxylic acid (1 g) was synthesized using solid phase, and was isolated as a white, fluffy solid.

ES/MS m/z 1146.7 (M+H)⁺.

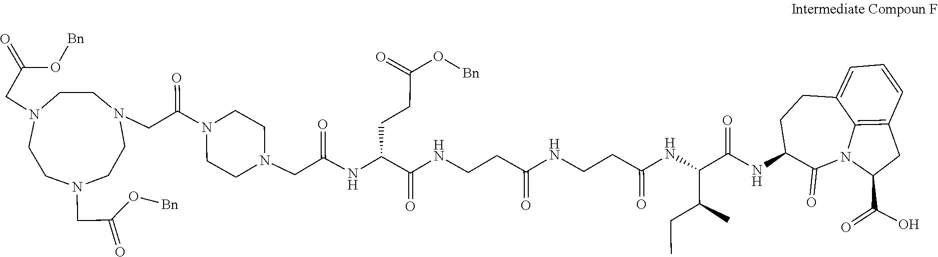 [SMILES: CC[C@H](C)[C@H](NC(=O)CCNC(=O)CCNC(=O)[C@@H](CCC(=O)OCc1ccccc1)NC(=O)CN1CCN(C(=O)CN2CCN(CC(=O)OCc3ccccc3)CCN(CC(=O)OCc3ccccc3)CC2)CC1)C(=O)N[C@H]1CCc2cccc3c2N(C1=O)[C@H](C(=O)O)C3]

Following procedure A, (3S,6S)-3-((6R,17S)-6-(2-(4-(2-(4,7-bis(2-(benzyloxy)-2-oxoethyl)-1,4,7-triazonan-1-yl)acetyl)piperazin-1-yl)acetamido)-17-((S)-sec-butyl)-3,7,11,15-tetraoxo-1-phenyl-2-oxa-8,12,16-triazaoctadecan-18-amido)-4-oxo-1,2,3,4,6,7-hexahydroazepino[3,2,1-hi]indole-6-carboxylic acid (184 mg) was synthesized using solid phase. For this specific example, one dipeptide intermediate, (R)-2-(2-(4-(((9H-fluoren-9-yl)methoxy)carbonyl)piperazin-1-yl)acetamido)-5-(benzyloxy)-5-oxopentanoic acid was used in the solid phase synthesis. It was isolated as a light-yellow solid.

ES/MS m/z 1313.1 (M+H)⁺.

### General Procedure for Precursor Synthesis:

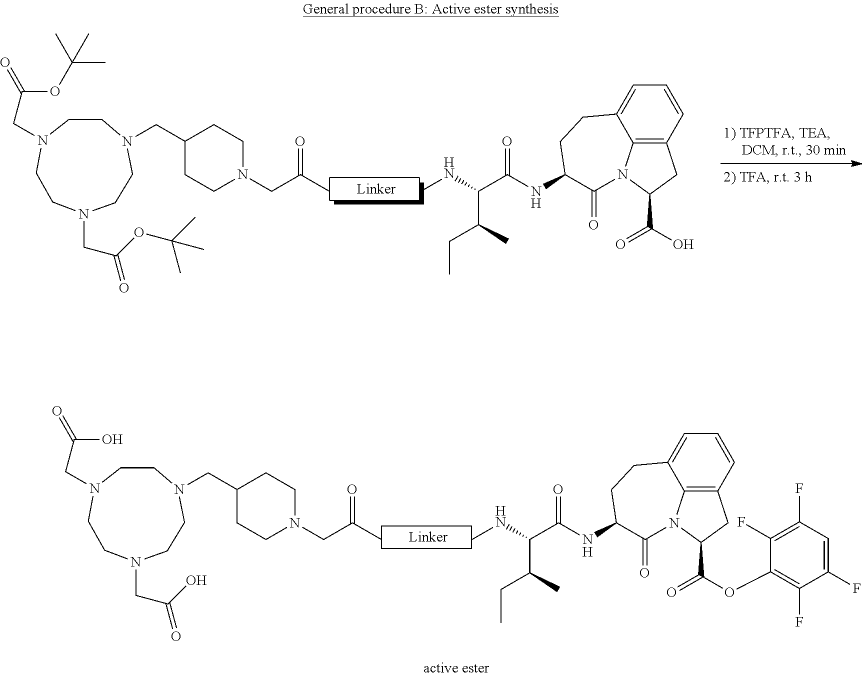 [SMILES: CC(=O)CN1CCC(CN2CCN(CC(=O)OC(C)(C)C)CCN(CC(=O)OC(C)(C)C)CC2)CC1 ; CC[C@H](C)[C@H](NC)C(=O)N[C@H]1CCc2cccc3c2N(C1=O)[C@H](C(=O)O)C3 ; CC(=O)CN1CCC(CN2CCN(CC(=O)O)CCN(CC(=O)O)CC2)CC1 ; CC[C@H](C)[C@H](NC)C(=O)N[C@H]1CCc2cccc3c2N(C1=O)[C@H](C(=O)Oc1c(F)c(F)cc(F)c1F)C3]

The peptide acid was dissolved in DCM (0.1 M) in a 20 mL vial equipped with a stirrer bar, followed by the addition of 2,3,5,6-tetrafluorophenyl trifluoroacetate (TFPTFA, 2 equiv.). The resulting light-yellow solution was cooled to 0° C. and was added triethylamine (2 equiv.). The yellow solution was allowed to warm to ambient temperature and was monitored by LCMS. TFA (same volume) was then added to the reaction mixture slowly, and the reaction was stirred at ambient temperature and was monitored by LCMS. Upon completion, the yellow reaction mixture was concentrated and purified by gold aq-C18 ISCO column using 0.1% formic acid in water and acetonitrile as the eluent. The proper fractions were collected and lyophilized to yield the active ester as white, fluffy solids.

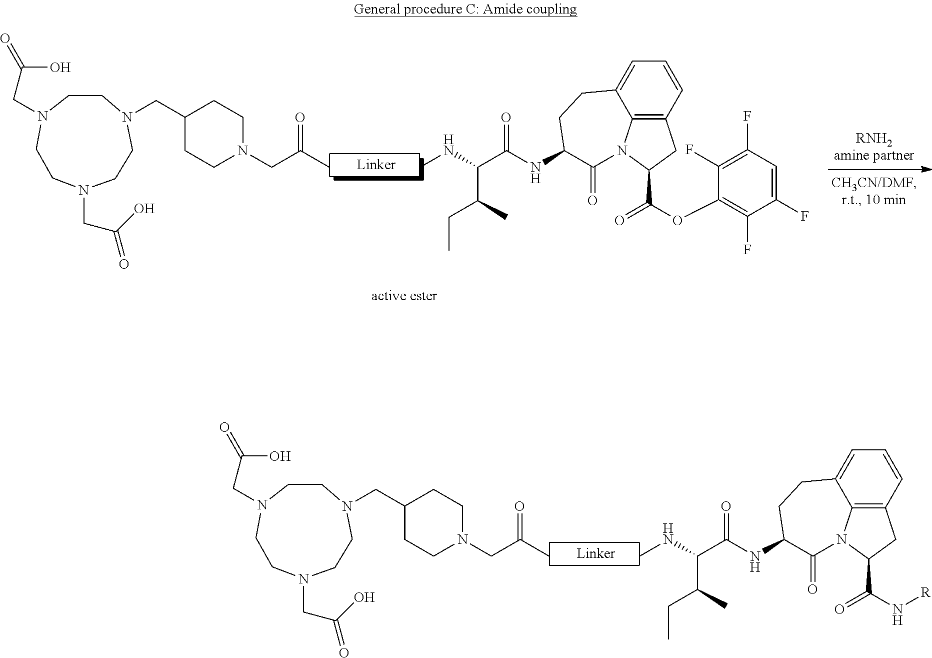

The Cbz-protected amine partner (>3 equiv.) was hydrogenated in ethyl acetate using H₂ and 20% Pd(OH)₂/C at ambient temperature, monitored by ¹H NMR analysis. The amine solution was concentrated and redissolved in DMF (1 mL) The light-yellow solution was transferred to a vial containing active ester (1 equiv.), and additional acetonitrile (2 mL) was added to help with the transfer. The reaction was then mixed with a drop of TEA and was monitored by LCMS. The clear, light-yellow reaction mixture was then concentrated and purified by Phenomenex Gemini C18 RP-HPLC preparatory column using 0.1% formic acid (or 20 mM ammonium acetate) in water and acetonitrile as the eluent. The proper fractions were collected and lyophilized to yield the prodrug precursor as off-white, fluffy solids (if purified by 0.1% FA in water), or light-yellow solids (if purified by 20 mM ammonium acetate in water).

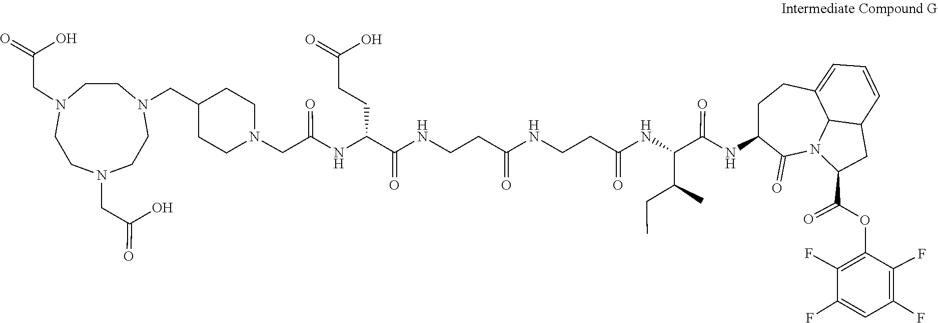 [SMILES: CC[C@H](C)[C@H](NC(=O)CCNC(=O)CCNC(=O)[C@@H](CCC(=O)O)NC(=O)CN1CCC(CN2CCN(CC(=O)O)CCN(CC(=O)O)CC2)CC1)C(=O)N[C@H]1CCC2=CC=CC3C[C@@H](C(=O)Oc4c(F)c(F)cc(F)c4F)N(C1=O)C23]

Following the general procedure B, 2,2′-(7-((1-((4R,15S,16S)-4-(2-carboxyethyl)-16-methyl-2,5,9,13-tetraoxo-15-(((3S,6S)-4-oxo-6-((2,3,5,6-tetrafluorophenoxy)carbonyl)-1,2,3,4,6,7-hexahydroazepino[3,2,1-hi]indol-3-yl)carbamoyl)-3,6,10,14-tetraazaoctadecyl)piperidin-4-yl)methyl)-1,4,7-triazonane-1,4-diyl)diacetic acid (72.3 mg) was synthesized from (3S,6S)-3-((2S,13R)-13-(2-(4-((4,7-bis(2-(tert-butoxy)-2-oxoethyl)-1,4,7-triazonan-1-yl)methyl)piperidin-1-yl)acetamido)-2-((S)-sec-butyl)-18,18-dimethyl-4,8,12,16-tetraoxo-17-oxa-3,7,11-triazanonadecanamido)-4-oxo-1,2,3,4,6,7-hexahydroazepino[3,2,1-hi]indole-6-carboxylic acid (100.0 mg), and was isolated as an off-white, fluffy solid.

ES/MS m/z 1161.4 (M+H)⁺.

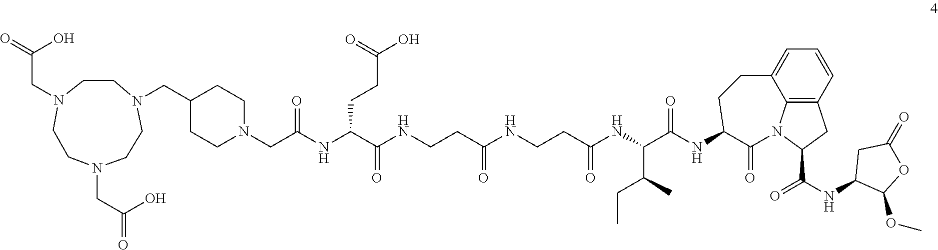 [SMILES: CC[C@H](C)[C@H](NC(=O)CCNC(=O)CCNC(=O)[C@@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)N[C@H]1CC(=O)O[C@H]1OC)C3]

Following the general procedure C, 2,2′-(7-((1-((4R,15S,16S)-4-(2-carboxyethyl)-15-(((3S,6S)-6-(((2R,3S)-2-methoxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperidin-4-yl)methyl)-1,4,7-triazonane-1,4-diyl)diacetic acid (25.0 mg) was synthesized from the corresponding active ester (40.0 mg), and was isolated as an off-white, fluffy solid.

ES/MS m/z 1126.5 (M+H)⁺.

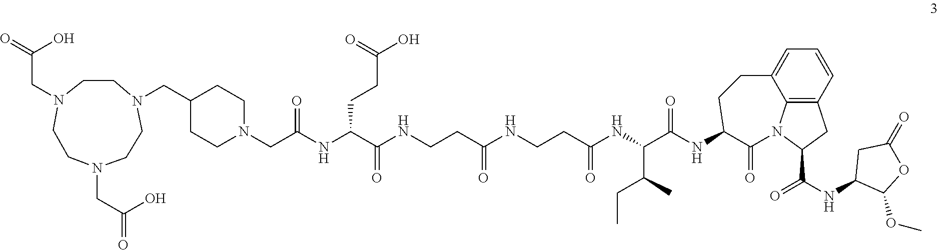 [SMILES: CC[C@H](C)[C@H](NC(=O)CCNC(=O)CCNC(=O)[C@@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)N[C@H]1CC(=O)O[C@@H]1OC)C3]

Following the general procedure C, 2,2′-(7-((1-((4R,15S,16S)-4-(2-carboxyethyl)-15-(((3S,6S)-6-(((2S,3S)-2-methoxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperidin-4-yl)methyl)-1,4,7-triazonane-1,4-diyl)diacetic acid (24.6 mg) was synthesized from the corresponding active ester (36.9 mg), and was isolated as an off-white, fluffy solid.

ES/MS m/z 1126.6 (M+H)⁺.

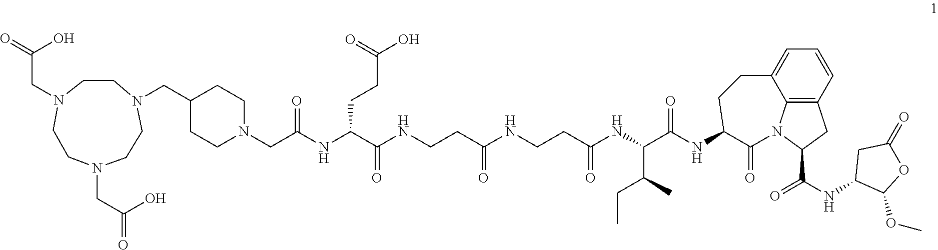 [SMILES: CC[C@H](C)[C@H](NC(=O)CCNC(=O)CCNC(=O)[C@@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)N[C@@H]1CC(=O)O[C@@H]1OC)C3]

To a solution of (3S,6S)-3-((6R,17S)-6-(2-(4-((4,7-bis(2-(benzyloxy)-2-oxoethyl)-1,4,7-triazonan-1-yl)methyl)piperidin-1-yl)acetamido)-17-((S)-sec-butyl)-3,7,11,15-tetraoxo-1-phenyl-2-oxa-8,12,16-triazaoctadecan-18-amido)-4-oxo-1,2,3,4,6,7-hexahydroazepino[3,2,1-hi]indole-6-carboxylic acid (180 mg, 0.12 mmol) in DMF (3 mL) was added ethyl cyano(hydroxyimino)acetate (50 mg, 0.34 mmol) and DIC (45 mg, 0.35 mmol) at 0° C. under N₂. The mixture was stirred at 0° C. for 0.5 h. (4R,5S)-4-amino-5-methoxydihydrofuran-2(3H)-one (34 mg, 0.26 mmol, prepared from the corresponding Cbz intermediate by catalytical hydrogenation) was added. The mixture was stirred at 20° C. for 12 h. The mixture was filtered, and the filtrates was purified by C18 prep-HPLC(formic acid in water-CH₃CN). The afforded fractions were combined and lyophilized. Dibenzyl 2,2′-(7-((1-((4R,15S,16S)-4-(3-(benzyloxy)-3-oxopropyl)-15-(((3S,6S)-6-(((2S,3R)-2-methoxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperidin-4-yl)methyl)-1,4,7-triazonane-1,4-diyl)diacetate (132 mg, 0.05 mmol) was obtained as white solid.

ES/MS m/z 1397.6 (M+H)⁺.

To a solution of dibenzyl 2,2′-(7-((1-((4R,15S,16S)-4-(3-(benzyloxy)-3-oxopropyl)-15-(((3S,6S)-6-(((2S,3R)-2-methoxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperidin-4-yl)methyl)-1,4,7-triazonane-1,4-diyl)diacetate (130 mg, 0.05 mmol) in methanol (1.2 mL) and tetrahydrofuran (4 mL) was added 10% Pd/C (50 mg). The reaction mixture was degassed and purged with H₂ for several times. The reaction mixture was stirred at 25° C. for 5 h under H₂ (15 Psi). The mixture was filtered, and the filtrates was purified by C18 prep-HPLC (formic acid in water-CH₃CN). The afforded fractions were combined and lyophilized. 2,2′-(7-((1-((4R,15S,16S)-4-(2-carboxyethyl)-15-(((3S,6S)-6-(((2S,3R)-2-methoxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperidin-4-yl)methyl)-1,4,7-triazonane-1,4-diyl)diacetic acid (40 mg, 0.036 mmol) was obtained as white solid.

ES/MS m/z 1126.9 (M+H)⁺.

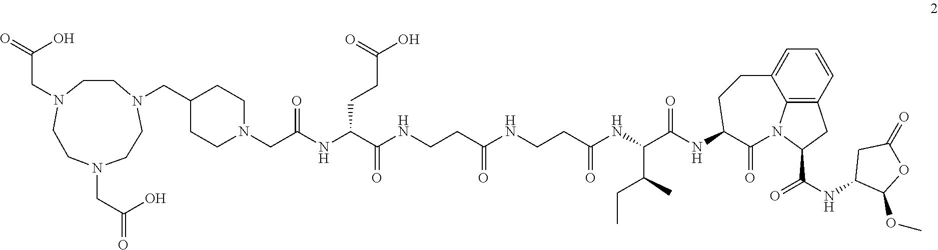 [SMILES: CC[C@H](C)[C@H](NC(=O)CCNC(=O)CCNC(=O)[C@@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)N[C@@H]1CC(=O)O[C@H]1OC)C3]

To a solution of compound (3S,6S)-3-((6R,17S)-6-(2-(4-((4,7-bis(2-(benzyloxy)-2-oxoethyl)-1,4,7-triazonan-1-yl)methyl)piperidin-1-yl)acetamido)-17-((S)-sec-butyl)-3,7,11,15-tetraoxo-1-phenyl-2-oxa-8,12,16-triazaoctadecan-18-amido)-4-oxo-1,2,3,4,6,7-hexahydroazepino[3,2,1-hi]indole-6-carboxylic acid (210 mg, 0.13 mmol) in DMF (3 mL) was added ethyl cyano(hydroxyimino)acetate (48 mg, 0.33 mmol) and DIC (43 mg, 0.33 mmol) at 0° C. under N₂. The mixture was stirred at 0° C. for 0.5 h. (4R,5R)-4-amino-5-methoxydihydrofuran-2(3H)-one (34 mg, 0.26 mmol, prepared from the corresponding Cbz intermediate by catalytical hydrogenation) was added. The mixture was stirred at 20° C. for 12 h. The crude product was purified by C18 prep-HPLC (formic acid in water-CH₃CN). The afforded fractions were combined and lyophilized. Dibenzyl 2,2′-(7-((1-((4R,15S,16S)-4-(3-(benzyloxy)-3-oxopropyl)-15-(((3S,6S)-6-(((2R,3R)-2-methoxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperidin-4-yl)methyl)-1,4,7-triazonane-1,4-diyl)diacetate (112 mg, 0.07 mmol) was obtained as white solid.

ES/MS m/z 1397.1 (M+H)⁺.

To a solution of dibenzyl 2,2′-(7-((1-((4R,15S,16S)-4-(3-(benzyloxy)-3-oxopropyl)-15-(((3S,6S)-6-(((2R,3R)-2-methoxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperidin-4-yl)methyl)-1,4,7-triazonane-1,4-diyl)diacetate (110 mg, 0.07 mmol) in methanol (1.2 mL) and tetrahydrofuran (4 mL) was added 10% Pd/C (50 mg) The reaction mixture was degassed and purged with H₂ for several times. The reaction mixture was stirred at 20° C. for 2 h under H₂ (15 Psi). The mixture was filtered, and the cake was washed with DCM (10 mL*3). The combined filtrates were concentrated. The residue was purified by C18 prep-HPLC (formic acid in water-CH₃CN). The afforded fractions were combined and lyophilized. 2,2′-(7-((1-((4R,15S,16S)-4-(2-carboxyethyl)-15-(((3S,6S)-6-(((2R,3R)-2-methoxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperidin-4-yl)methyl)-1,4,7-triazonane-1,4-diyl)diacetic acid (26 mg, 0.02 mmol) was obtained as white solid.

ES/MS m/z 1126.9 (M+H)⁺.

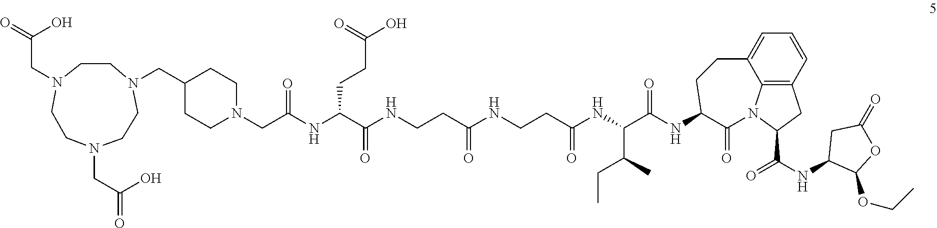 [SMILES: CCO[C@@H]1OC(=O)C[C@@H]1NC(=O)[C@@H]1Cc2cccc3c2N1C(=O)[C@@H](NC(=O)[C@@H](NC(=O)CCNC(=O)CCNC(=O)[C@@H](CCC(=O)O)NC(=O)CN1CCC(CN2CCN(CC(=O)O)CCN(CC(=O)O)CC2)CC1)[C@@H](C)CC)CC3]

Following the general procedure C, 2,2′-(7-((1-((4R,15S,16S)-4-(2-carboxyethyl)-15-(((3S,6S)-6-(((2R,3S)-2-ethoxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperidin-4-yl)methyl)-1,4,7-triazonane-1,4-diyl)diacetic acid (21.9 mg) was synthesized from the corresponding active ester (33.0 mg), and was isolated as an off-white, fluffy solid.

ES/MS m/z 1140.6 (M+H)⁺.

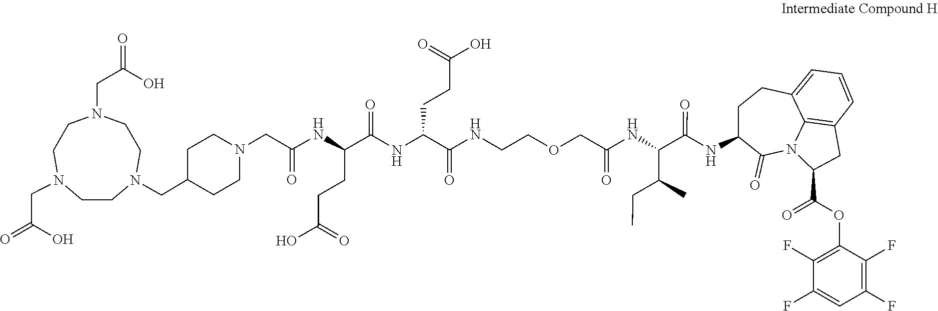 [SMILES: CC[C@H](C)[C@H](NC(=O)COCCNC(=O)[C@@H](CCC(=O)O)NC(=O)[C@@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)Oc1c(F)c(F)cc(F)c1F)C3]

Following the general procedure B, 2,2′-(7-((1-((4R,7R,16S,17S)-4,7-bis(2-carboxyethyl)-17-methyl-2,5,8,14-tetraoxo-16-(((3S,6S)-4-oxo-6-((2,3,5,6-tetrafluorophenoxy)carbonyl)-1,2,3,4,6,7-hexahydroazepino[3,2,1-hi]indol-3-yl)carbamoyl)-12-oxa-3,6,9,15-tetraazanonadecyl)piperidin-4-yl)methyl)-1,4,7-triazonane-1,4-diyl)diacetic acid (85.1 mg) was synthesized from (3S,6S)-3-((2S,11R,14R)-14-(2-(4-((4,7-bis(2-(tert-butoxy)-2-oxoethyl)-1,4,7-triazonan-1-yl)methyl)piperidin-1-yl)acetamido)-11-(3-(tert-butoxy)-3-oxopropyl)-2-((S)-sec-butyl)-19,19-dimethyl-4,10,13,17-tetraoxo-6,18-dioxa-3,9,12-triazaicosanamido)-4-oxo-1,2,3,4,6,7-hexahydroazepino[3,2,1-hi]indole-6-carboxylic acid (191.2 mg), and was isolated as an off-white, fluffy solid.

ES/MS m/z 1249.6 (M+H)⁺.

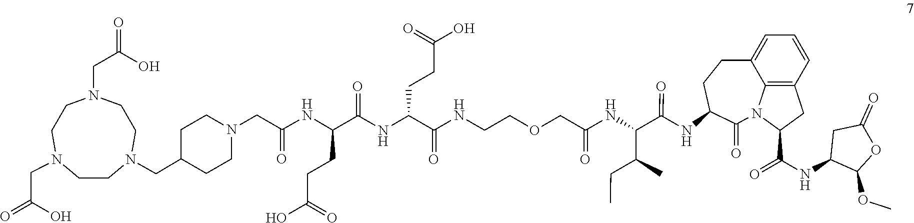 [SMILES: CC[C@H](C)[C@H](NC(=O)COCCNC(=O)[C@@H](CCC(=O)O)NC(=O)[C@@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)N[C@H]1CC(=O)O[C@H]1OC)C3]

Following the general procedure C, 2,2′-(7-((1-((4R,7R,16S,17S)-4,7-bis(2-carboxyethyl)-16-(((3S,6S)-6-(((2R,3S)-2-methoxy-5-oxotetrahydrofuran-3-yl)carbamoyl)-4-oxo-1,2,3,4,6,7-hexahydroazepino[3,2,1-hi]indol-3-yl)carbamoyl)-17-methyl-2,5,8,14-tetraoxo-12-oxa-3,6,9,15-tetraazanonadecyl)piperidin-4-yl)methyl)-1,4,7-triazonane-1,4-diyl)diacetic acid (61.9 mg) was synthesized from the corresponding active ester (65.0 mg), and was isolated as an off-white, fluffy solid.

ES/MS m/z 1214.8 (M+H)⁺.

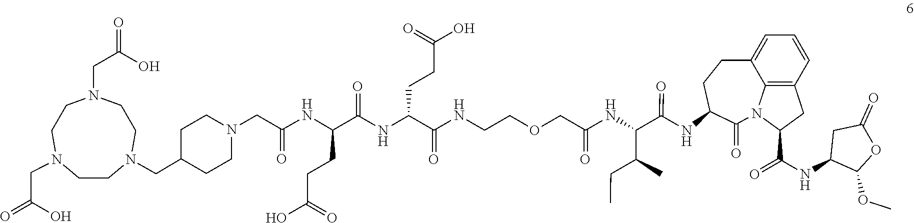 [SMILES: CC[C@H](C)[C@H](NC(=O)COCCNC(=O)[C@@H](CCC(=O)O)NC(=O)[C@@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)N[C@H]1CC(=O)O[C@@H]1OC)C3]

Following the general procedure C, 2,2′-(7-((1-((4R,7R,16S,17S)-4,7-bis(2-carboxyethyl)-16-(((3S,6S)-6-(((2S,3S)-2-methoxy-5-oxotetrahydrofuran-3-yl)carbamoyl)-4-oxo-1,2,3,4,6,7-hexahydroazepino[3,2,1-hi]indol-3-yl)carbamoyl)-17-methyl-2,5,8,14-tetraoxo-12-oxa-3,6,9,15-tetraazanonadecyl)piperidin-4-yl)methyl)-1,4,7-triazonane-1,4-diyl)diacetic acid (14.1 mg) was synthesized from the corresponding active ester (25.3 mg), and was isolated as an off-white, fluffy solid.

ES/MS m/z 1214.8 (M+H)⁺.

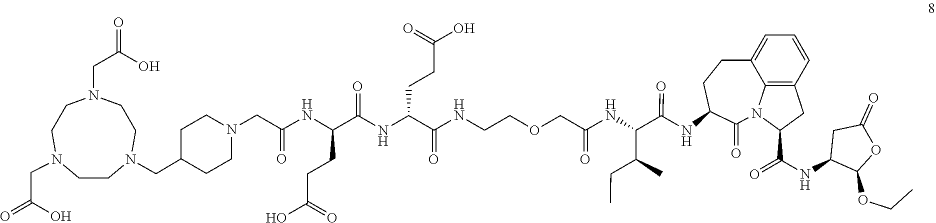 [SMILES: CCO[C@@H]1OC(=O)C[C@@H]1NC(=O)[C@@H]1Cc2cccc3c2N1C(=O)[C@@H](NC(=O)[C@@H](NC(=O)COCCNC(=O)[C@@H](CCC(=O)O)NC(=O)[C@@H](CCC(=O)O)NC(=O)CN1CCC(CN2CCN(CC(=O)O)CCN(CC(=O)O)CC2)CC1)[C@@H](C)CC)CC3]

Following the general procedure C, 2,2′-(7-((1-((4R,7R,16S,17S)-4,7-bis(2-carboxyethyl)-16-(((3S,6S)-6-(((2R,3S)-2-ethoxy-5-oxotetrahydrofuran-3-yl)carbamoyl)-4-oxo-1,2,3,4,6,7-hexahydroazepino[3,2,1-hi]indol-3-yl)carbamoyl)-17-methyl-2,5,8,14-tetraoxo-12-oxa-3,6,9,15-tetraazanonadecyl)piperidin-4-yl)methyl)-1,4,7-triazonane-1,4-diyl)diacetic acid (26.5 mg) was synthesized from the corresponding active ester (43.0 mg), and was isolated as an off-white, fluffy solid.

ES/MS m/z 1228.9 (M+H)⁺.

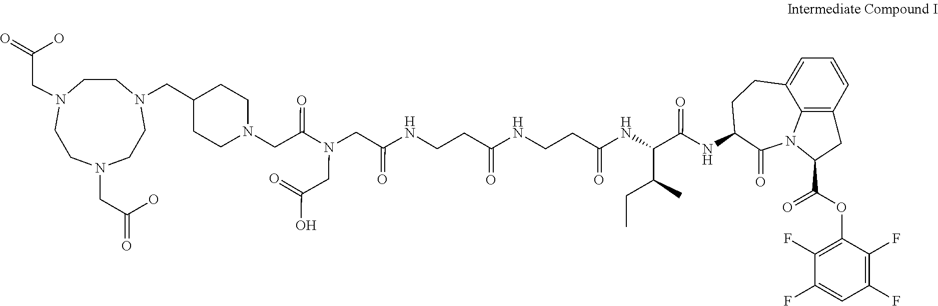 [SMILES: CC[C@H](C)[C@H](NC(=O)CCNC(=O)CCNC(=O)CN(CC(=O)O)C(=O)CN1CCC(CN2CCN(CC(=O)O)CCN(CC(=O)O)CC2)CC1)C(=O)N[C@H]1CCc2cccc3c2N(C1=O)[C@H](C(=O)Oc1c(F)c(F)cc(F)c1F)C3]

Following the general procedure B, 2,2′-(7-((1-((15S,16S)-3-(carboxymethyl)-16-methyl-2,5,9,13-tetraoxo-15-(((3S,6S)-4-oxo-6-((2,3,5,6-tetrafluorophenoxy)carbonyl)-1,2,3,4,6,7-hexahydroazepino[3,2,1-hi]indol-3-yl)carbamoyl)-3,6,10,14-tetraazaoctadecyl)piperidin-4-yl)methyl)-1,4,7-triazonane-1,4-diyl)diacetic acid (91.8 mg) was synthesized from (3S,6S)-3-((S)-6-(2-(4-((4,7-bis(2-(tert-butoxy)-2-oxoethyl)-1,4,7-triazonan-1-yl)methyl)piperidin-1-yl)acetyl)-18-((S)-sec-butyl)-2,2-dimethyl-4,8,12,16-tetraoxo-3-oxa-6,9,13,17-tetraazanonadecan-19-amido)-4-oxo-1,2,3,4,6,7-hexahydroazepino[3,2,1-hi]indole-6-carboxylic acid (180.0 mg), and was isolated as an off-white, fluffy solid.

ES/MS m/z 1147.5 (M+H)⁺.

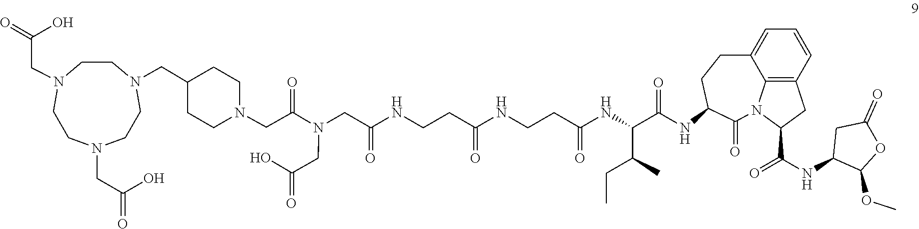 [SMILES: CC[C@H](C)[C@H](NC(=O)CCNC(=O)CCNC(=O)CN(CC(=O)O)C(=O)CN1CCC(CN2CCN(CC(=O)O)CCN(CC(=O)O)CC2)CC1)C(=O)N[C@H]1CCc2cccc3c2N(C1=O)[C@H](C(=O)N[C@H]1CC(=O)O[C@H]1OC)C3]

Following the general procedure C, 2,2′-(7-((1-((15S,16S)-3-(carboxymethyl)-15-(((3S,6S)-6-(((2R,3S)-2-methoxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperidin-4-yl)methyl)-1,4,7-triazonane-1,4-diyl)diacetic acid (43.2 mg) was synthesized from the corresponding active ester (50.0 mg), and was isolated as an off-white, fluffy solid.

ES/MS m/z 1112.5 (M+H)⁺.

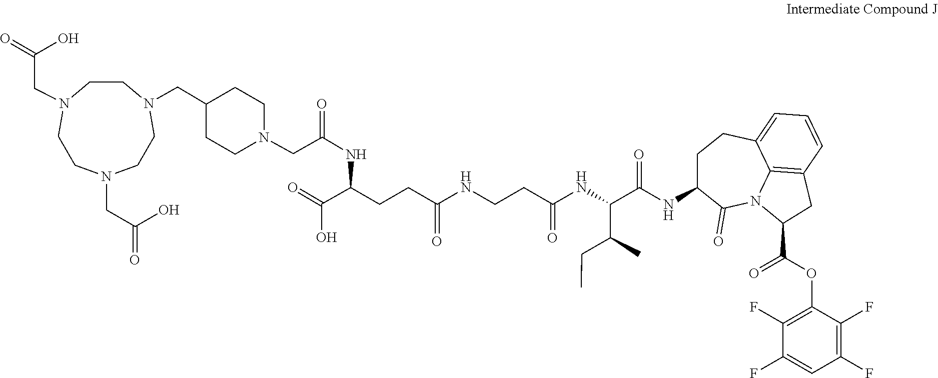 [SMILES: CC[C@H](C)[C@H](NC(=O)CCNC(=O)CC[C@H](NC(=O)CN1CCC(CN2CCN(CC(=O)O)CCN(CC(=O)O)CC2)CC1)C(=O)O)C(=O)N[C@H]1CCc2cccc3c2N(C1=O)[C@H](C(=O)Oc1c(F)c(F)cc(F)c1F)C3]

Following procedure B, 2,2′-(7-((1-(2-(((S)-1-carboxy-4-((3-(((2S,3S)-3-methyl-1-oxo-1-(((3S,6S)-4-oxo-6-((2,3,5,6-tetrafluorophenoxy)carbonyl)-1,2,3,4,6,7-hexahydroazepino[3,2,1-hi]indol-3-yl)amino)pentan-2-yl)amino)-3-oxopropyl)amino)-4-oxobutyl)amino)-2-oxoethyl)piperidin-4-yl)methyl)-1,4,7-triazonane-1,4-diyl)diacetic acid (83.0 mg) was synthesized from (3S,6S)-3-((2S,3S)-2-(3-((S)-4-(2-(4-((4,7-bis(2-(tert-butoxy)-2-oxoethyl)-1,4,7-triazonan-1-yl)methyl)piperidin-1-yl)acetamido)-5-(tert-butoxy)-5-oxopentanamido)propanamido)-3-methylpentanamido)-4-oxo-1,2,3,4,6,7-hexahydroazepino[3,2,1-hi]indole-6-carboxylic acid (100.0 mg), and was isolated as an off-white, fluffy solid.

ES/MS m/z 1090.5 (M+H)⁺.

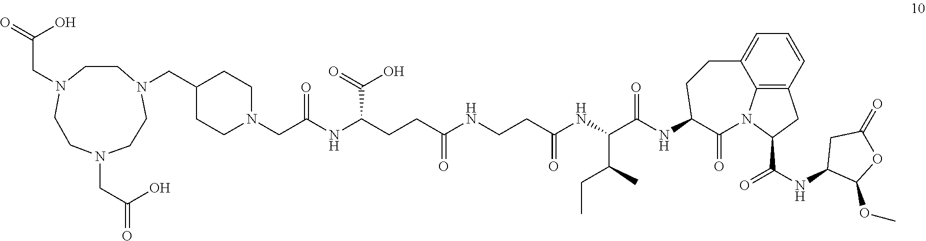 [SMILES: CC[C@H](C)[C@H](NC(=O)CCNC(=O)CC[C@H](NC(=O)CN1CCC(CN2CCN(CC(=O)O)CCN(CC(=O)O)CC2)CC1)C(=O)O)C(=O)N[C@H]1CCc2cccc3c2N(C1=O)[C@H](C(=O)N[C@H]1CC(=O)O[C@H]1OC)C3]

Following procedure C, 2,2′-(7-((1-(2-(((S)-1-carboxy-4-((3-(((2S,3S)-1-(((3S,6S)-6-(((2R,3S)-2-methoxy-5-oxotetrahydrofuran-3-yl)carbamoyl)-4-oxo-1,2,3,4,6,7-hexahydroazepino[3,2,1-hi]indol-3-yl)amino)-3-methyl-1-oxopentan-2-yl)amino)-3-oxopropyl)amino)-4-oxobutyl)amino)-2-oxoethyl)piperidin-4-yl)methyl)-1,4,7-triazonane-1,4-diyl)diacetic acid (24.8 mg) was synthesized from the corresponding active ester (83.0 mg), and was isolated as a light yellow powder.

ES/MS m/z 1055.5 (M+H)⁺.

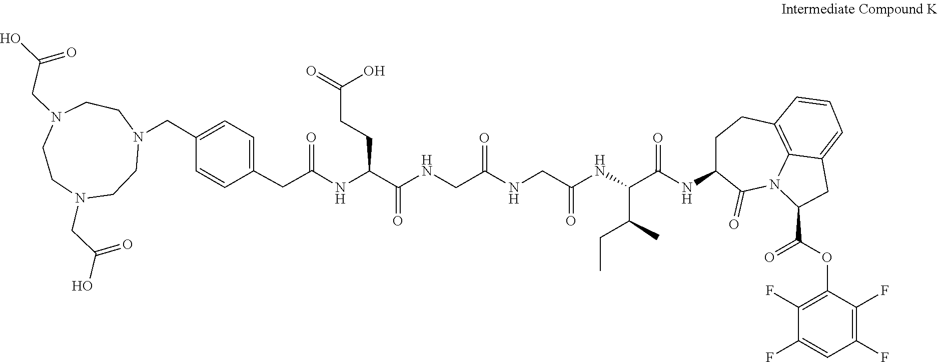 [SMILES: CC[C@H](C)[C@H](NC(=O)CNC(=O)CNC(=O)[C@H](CCC(=O)O)NC(=O)Cc1ccc(CN2CCN(CC(=O)O)CCN(CC(=O)O)CC2)cc1)C(=O)N[C@H]1CCc2cccc3c2N(C1=O)[C@H](C(=O)Oc1c(F)c(F)cc(F)c1F)C3]

Following procedure B, 2,2′-(7-(4-((4S,13S,14S)-4-(2-carboxyethyl)-14-methyl-2,5,8,11-tetraoxo-13-(((3S,6S)-4-oxo-6-((2,3,5,6-tetrafluorophenoxy)carbonyl)-1,2,3,4,6,7-hexahydroazepino[3,2,1-hi]indol-3-yl)carbamoyl)-3,6,9,12-tetraazahexadecyl)benzyl)-1,4,7-triazonane-1,4-diyl)diacetic acid (20.0 mg) was synthesized from (3S,6S)-3-((2S,11S)-11-(2-(4-((4,7-bis(2-(tert-butoxy)-2-oxoethyl)-1,4,7-triazonan-1-yl)methyl)phenyl)acetamido)-2-((S)-sec-butyl)-16,16-dimethyl-4,7,10,14-tetraoxo-15-oxa-3,6,9-triazaheptadecanamido)-4-oxo-1,2,3,4,6,7-hexahydroazepino[3,2,1-hi]indole-6-carboxylic acid (30.6 mg), and was isolated as an white, fluffy solid.

ES/MS m/z 1126.4 (M+H)⁺.

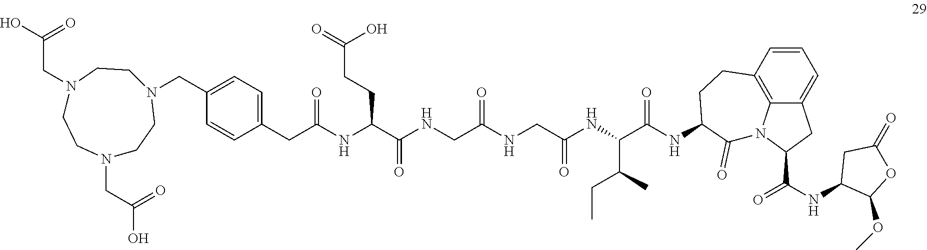 [SMILES: CC[C@H](C)[C@H](NC(=O)CNC(=O)CNC(=O)[C@H](CCC(=O)O)NC(=O)Cc1ccc(CN2CCN(CC(=O)O)CCN(CC(=O)O)CC2)cc1)C(=O)N[C@H]1CCc2cccc3c2N(C1=O)[C@H](C(=O)N[C@H]1CC(=O)O[C@H]1OC)C3]

Following the general procedure C, 2,2′-(7-(4-((4S,13S,14S)-4-(2-carboxyethyl)-13-(((3S,6S)-6-(((2R,3S)-2-methoxy-5-oxotetrahydrofuran-3-yl)carbamoyl)-4-oxo-1,2,3,4,6,7-hexahydroazepino[3,2,1-hi]indol-3-yl)carbamoyl)-14-methyl-2,5,8,11-tetraoxo-3,6,9,12-tetraazahexadecyl)benzyl)-1,4,7-triazonane-1,4-diyl)diacetic acid (12.0 mg) was synthesized from the corresponding active ester (19.0 mg), and was isolated as an off-white, fluffy solid.

ES/MS m/z 1091.5 (M+H)⁺.

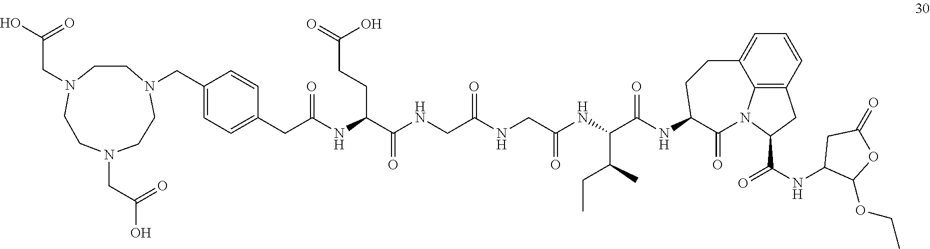 [SMILES: CCOC1OC(=O)CC1NC(=O)[C@@H]1Cc2cccc3c2N1C(=O)[C@@H](NC(=O)[C@@H](NC(=O)CNC(=O)CNC(=O)[C@H](CCC(=O)O)NC(=O)Cc1ccc(CN2CCN(CC(=O)O)CCN(CC(=O)O)CC2)cc1)[C@@H](C)CC)CC3]

A stirred solution of 2,2′-(7-(4-((4S,13S,14S)-4-(2-carboxyethyl)-13-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-14-methyl-2,5,8,11-tetraoxo-3,6,9,12-tetraazahexadecyl)benzyl)-1,4,7-triazonane-1,4-diyl)diacetic acid (15.0 mg) in a mixture of 3.0 mL ethanol and 0.75 mL ethyl orthoacetate with ~20 uL TFA was heated to 65° C. overnight. The solution was concentrated, and the residue was redissolved in 10% DMSO in H₂O. The mixture was purified by HPLC (0.1% FA in H₂O to CH₃CN, 0-60%) to afford 2,2′-(7-(4-((4S,13S,14S)-4-(2-carboxyethyl)-13-(((3S,6S)-6-((2-ethoxy-5-oxotetrahydrofuran-3-yl)carbamoyl)-4-oxo-1,2,3,4,6,7-hexahydroazepino[3,2,1-hi]indol-3-yl)carbamoyl)-14-methyl-2,5,8,11-tetraoxo-3,6,9,12-tetraazahexadecyl)benzyl)-1,4,7-triazonane-1,4-diyl)diacetic acid (2.5 mg) as an off white solid.

ES/MS m/z 1105.5 (M+H)⁺.

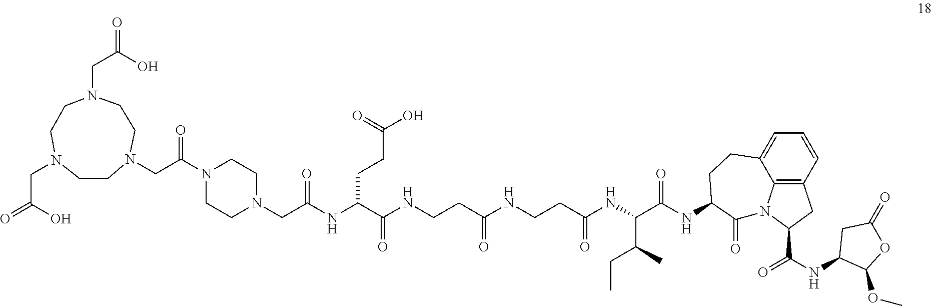 [SMILES: CC[C@H](C)[C@H](NC(=O)CCNC(=O)CCNC(=O)[C@@H](CCC(=O)O)NC(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)N[C@H]1CC(=O)O[C@H]1OC)C3]

To a solution of compound (3S,6S)-3-((6R,17S)-6-(2-(4-(2-(4,7-bis(2-(benzyloxy)-2-oxoethyl)-1,4,7-triazonan-1-yl)acetyl)piperazin-1-yl)acetamido)-17-((S)-sec-butyl)-3,7,11,15-tetraoxo-1-phenyl-2-oxa-8,12,16-triazaoctadecan-18-amido)-4-oxo-1,2,3,4,6,7-hexahydroazepino[3,2,1-hi]indole-6-carboxylic acid (100 mg, 0.069 mmol) and ethyl cyanohydroxyiminoacetate (28 mg, 0.19 mmol) in DMF (1 mL) was added N,N-diisopropylcarbodiimide (25 mg, 0.19 mmol) at 0° C. The reaction mixture was stirred at 0° C. for 0.5 h under N₂. Then (4S,5R)-4-amino-5-methoxydihydrofuran-2(3H)-one (25 mg, 0.19 mmol) in DMF (2 mL) was added. The reaction mixture was stirred at 20° C. for 16 h under N₂. The mixture was filtered, and the filtrates was purified by prep-HPLC (formic acid in water-CH₃CN). The afforded fractions were combined and lyophilized to afford dibenzyl 2,2′-(7-(2-(4-((4R,15S,16S)-4-(3-(benzyloxy)-3-oxopropyl)-15-(((3S,6S)-6-(((2R,3S)-2-methoxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperazin-1-yl)-2-oxoethyl)-1,4,7-triazonane-1,4-diyl)diacetate (41 mg, 0.025 mmol) as a white solid.

ES/MS m/z 1426.7 (M+H)⁺.

To a solution of dibenzyl 2,2′-(7-(2-(4-((4R,15S,16S)-4-(3-(benzyloxy)-3-oxopropyl)-15-(((3S,6S)-6-(((2R,3S)-2-methoxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperazin-1-yl)-2-oxoethyl)-1,4,7-triazonane-1,4-diyl)diacetate (41 mg, 0.025 mmol) in tetrahydrofuran (2 mL) and methanol (0.5 mL) was added Pd/C (15 mg). The reaction mixture was degassed and purged with H₂ for several times. The reaction mixture was stirred under H₂ atmosphere pressure at 20° C. for 3 h under N₂. The mixture was filtered, and the filtrates was concentrated under reduced pressure. The crude product was purified by C18 prep-HPLC (formic acid water-CH₃CN). The afforded fractions were combined and lyophilized to afford 2,2′-(7-(2-(4-((4R,15S,16S)-4-(2-carboxyethyl)-15-(((3S,6S)-6-(((2R,3S)-2-methoxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperazin-1-yl)-2-oxoethyl)-1,4,7-triazonane-1,4-diyl)diacetic acid (12.4 mg, 0.01 mmol).

ES/MS m/z 1156.0 (M+H)⁺.

AlF Complexation

General procedure D: To a reaction vial containing peptide precursor and a stir bar, was added equal equivalent (1.5-3.0 equiv. relative to peptide) of 20 mM AlCl₃ in 0.1 M NaOAc (pH~4.5) and 100 mM NaF in H₂O. Then acetonitrile (0-34% of the total reaction volume) was added. The mixture was heated to 100° C. for 15-30 mins. Acetonitrile was removed under reduced pressure, and the aqueous solution was purified by either gold aq-C18 ISCO column or Phenomenex Gemini C18 RP-HPLC preparatory column (using 0.1% formic acid (or 20 mM ammonium acetate) in water and acetonitrile as eluents). The proper fractions were collected and lyophilized to afford the peptide AlF complexes as white, fluffy solids (purified by 0.1% FA in water), or light-yellow solids (purified by 20 mM ammonium acetate in water).

General procedure E: To a 4 mL vial containing 100 μL peptide precursor solution (~200 nmol, 2 mg/mL in 1N Acetate buffer, pH 3.5) was added 60 μL AlCl₃ solution [144 nmol, 2.4 mM in WIC (Water for Ion Chromatography)], 10 μL NaF (100 nmol, 10 mM in WIC) and 230 μL acetonitrile (HPLC grade). The mixture was heated in the heating block on 105° C. for 15 minutes before diluted by 1.6 mL WIC. The reaction mixture was then analyzed by LCMS.

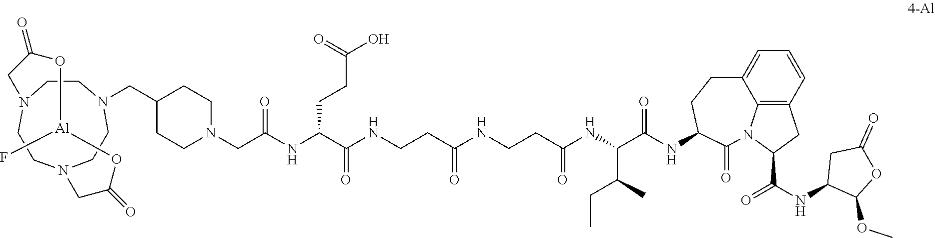

Following the general procedure D, 2,2′-(7-((1-((4R,15S,16S)-4-(2-carboxyethyl)-15-(((3S,6S)-6-(((2R,3S)-2-methoxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperidin-4-yl)methyl)-1,4,7-triazonane-1,4-diyl)diacetic acid, aluminum fluoride complex (1:1) (14.6 mg) was synthesized from 2,2′-(7-((1-((4R,15S,16S)-4-(2-carboxyethyl)-15-(((3S,6S)-6-(((2R,3S)-2-methoxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperidin-4-yl)methyl)-1,4,7-triazonane-1,4-diyl)diacetic acid (20.4 mg), and was isolated as a white, fluffy solid.

ES/MS m/z 1170.7 (M+H)⁺.

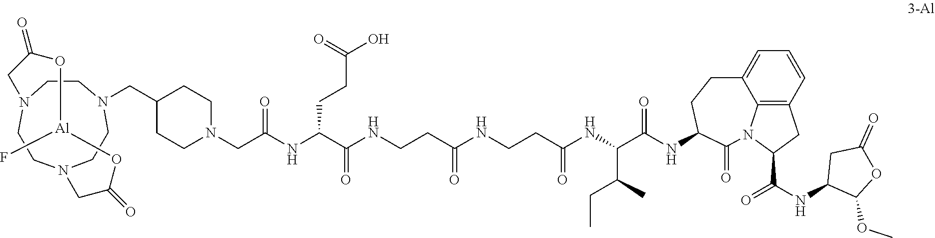

Following the general procedure E, 2,2′-(7-((1-((4R,15S,16S)-4-(2-carboxyethyl)-15-(((3S,6S)-6-(((2S,3S)-2-methoxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperidin-4-yl)methyl)-1,4,7-triazonane-1,4-diyl)diacetic acid, aluminum fluoride complex (1:1) was detected by HPLC.

ES/MS m/z 1192.6 (M+Na)⁺.

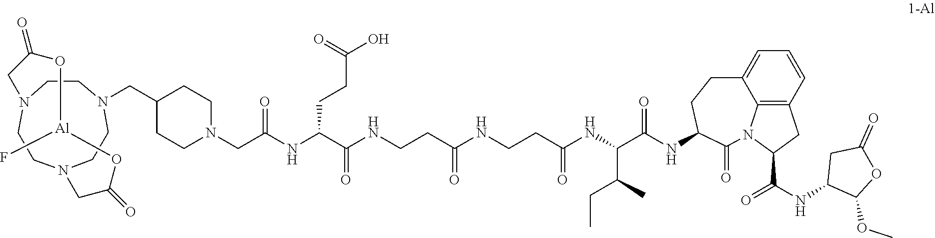

Following the general procedure E, 2,2′-(7-((1-((4R,15S,16S)-4-(2-carboxyethyl)-15-(((3S,6S)-6-(((2S,3R)-2-methoxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperidin-4-yl)methyl)-1,4,7-triazonane-1,4-diyl)diacetic acid, aluminum fluoride complex (1:1) was detected by HPLC.

ES/MS m/z 1170.6 (M+H)⁺.

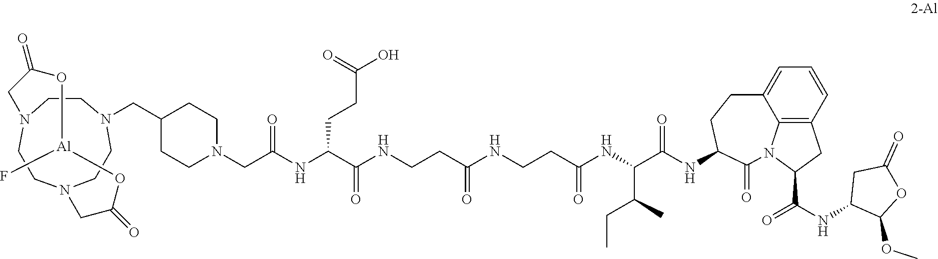

Following the general procedure E, 2,2′-(7-((1-((4R,15S,16S)-4-(2-carboxyethyl)-15-(((3S,6S)-6-(((2R,3R)-2-methoxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperidin-4-yl)methyl)-1,4,7-triazonane-1,4-diyl)diacetic acid, aluminum fluoride complex (1:1) was detected by HPLC.

ES/MS m/z 1170.6 (M+H)⁺.

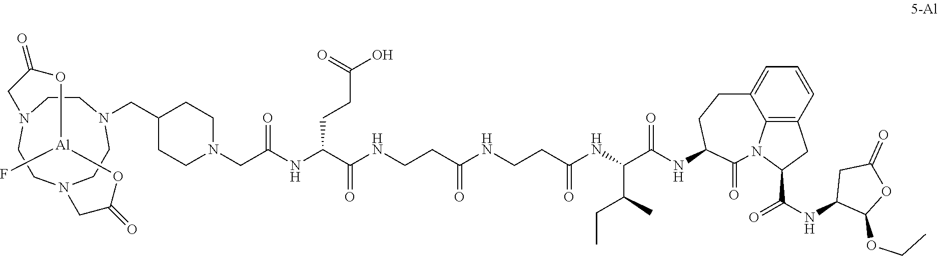

Following the general procedure D, 2,2′-(7-((1-((4R,15S,16S)-4-(2-carboxyethyl)-15-(((3S,6S)-6-(((2R,3S)-2-ethoxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperidin-4-yl)methyl)-1,4,7-triazonane-1,4-diyl)diacetic acid, aluminum fluoride complex (1:1) (5.0 mg) was synthesized from 2,2′-(7-((1-((4R,15S,16S)-4-(2-carboxyethyl)-15-(((3S,6S)-6-(((2R,3S)-2-ethoxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperidin-4-yl)methyl)-1,4,7-triazonane-1,4-diyl)diacetic acid (10.4 mg), and was isolated as a white, fluffy solid.

ES/MS m/z 1184.6 (M+H)⁺.

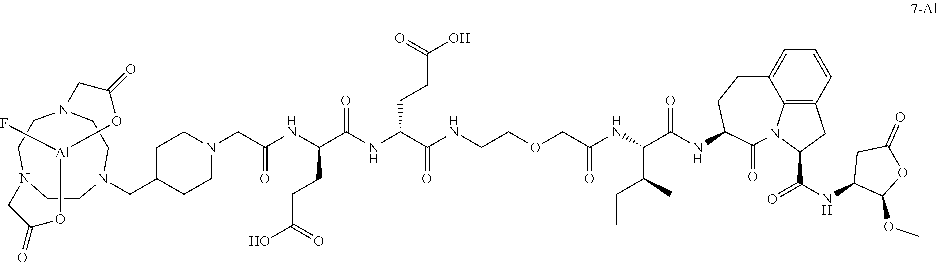

Following the general procedure D, 2,2′-(7-((1-((4R,7R,16S,17S)-4,7-bis(2-carboxyethyl)-16-(((3S,6S)-6-(((2R,3S)-2-methoxy-5-oxotetrahydrofuran-3-yl)carbamoyl)-4-oxo-1,2,3,4,6,7-hexahydroazepino[3,2,1-hi]indol-3-yl)carbamoyl)-17-methyl-2,5,8,14-tetraoxo-12-oxa-3,6,9,15-tetraazanonadecyl)piperidin-4-yl)methyl)-1,4,7-triazonane-1,4-diyl)diacetic acid, aluminum fluoride complex (1:1) (10.6 mg) was synthesized from 2,2′-(7-((1-((4R,7R,16S,17S)-4,7-bis(2-carboxyethyl)-16-(((3S,6S)-6-(((2R,3S)-2-methoxy-5-oxotetrahydrofuran-3-yl)carbamoyl)-4-oxo-1,2,3,4,6,7-hexahydroazepino[3,2,1-hi]indol-3-yl)carbamoyl)-17-methyl-2,5,8,14-tetraoxo-12-oxa-3,6,9,15-tetraazanonadecyl)piperidin-4-yl)methyl)-1,4,7-triazonane-1,4-diyl)diacetic acid (20.0 mg), and was isolated as a white, fluffy solid.

ES/MS m/z 1258.6 (M+H)⁺.

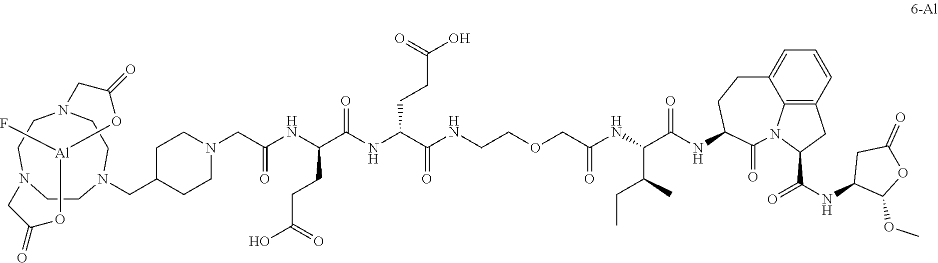

Following the general procedure D, 2,2′-(7-((1-((4R,7R,16S,17S)-4,7-bis(2-carboxyethyl)-16-(((3S,6S)-6-(((2S,3S)-2-methoxy-5-oxotetrahydrofuran-3-yl)carbamoyl)-4-oxo-1,2,3,4,6,7-hexahydroazepino[3,2,1-hi]indol-3-yl)carbamoyl)-17-methyl-2,5,8,14-tetraoxo-12-oxa-3,6,9,15-tetraazanonadecyl)piperidin-4-yl)methyl)-1,4,7-triazonane-1,4-diyl)diacetic acid, aluminum fluoride complex (1:1) (1.1 mg) was synthesized from 2,2′-(7-((1-((4R,7R,16S,17S)-4,7-bis(2-carboxyethyl)-16-(((3S,6S)-6-(((2S,3S)-2-methoxy-5-oxotetrahydrofuran-3-yl)carbamoyl)-4-oxo-1,2,3,4,6,7-hexahydroazepino[3,2,1-hi]indol-3-yl)carbamoyl)-17-methyl-2,5,8,14-tetraoxo-12-oxa-3,6,9,15-tetraazanonadecyl)piperidin-4-yl)methyl)-1,4,7-triazonane-1,4-diyl)diacetic acid (4.8 mg), and was isolated as a white, fluffy solid.

ES/MS m/z 1258.6 (M+H)⁺.

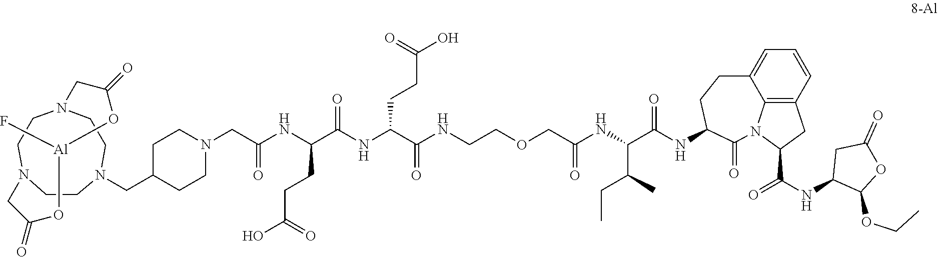

Following the general procedure D, 2,2′-(7-((1-((4R,7R,16S,17S)-4,7-bis(2-carboxyethyl)-16-(((3S,6S)-6-(((2R,3S)-2-ethoxy-5-oxotetrahydrofuran-3-yl)carbamoyl)-4-oxo-1,2,3,4,6,7-hexahydroazepino[3,2,1-hi]indol-3-yl)carbamoyl)-17-methyl-2,5,8,14-tetraoxo-12-oxa-3,6,9,15-tetraazanonadecyl)piperidin-4-yl)methyl)-1,4,7-triazonane-1,4-diyl)diacetic acid, aluminum fluoride complex (1:1) (3.3 mg) was synthesized from 2,2′-(7-((1-((4R,7R,16S,17S)-4,7-bis(2-carboxyethyl)-16-(((3S,6S)-6-(((2R,3S)-2-ethoxy-5-oxotetrahydrofuran-3-yl)carbamoyl)-4-oxo-1,2,3,4,6,7-hexahydroazepino[3,2,1-hi]indol-3-yl)carbamoyl)-17-methyl-2,5,8,14-tetraoxo-12-oxa-3,6,9,15-tetraazanonadecyl)piperidin-4-yl)methyl)-1,4,7-triazonane-1,4-diyl)diacetic acid (10.5 mg), and was isolated as a white, fluffy solid.

ES/MS m/z 1272.6 (M+H)⁺.

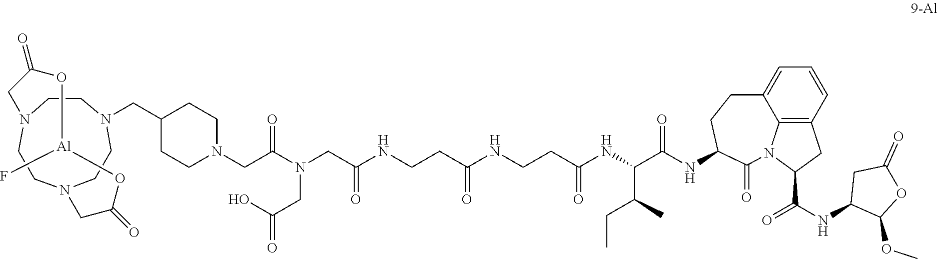

Following the general procedure D, 2,2′-(7-((1-((15S,16S)-3-(carboxymethyl)-15-(((3S,6S)-6-(((2R,3S)-2-methoxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperidin-4-yl)methyl)-1,4,7-triazonane-1,4-diyl)diacetic acid, aluminum fluoride complex (1:1) (7.5 mg) was synthesized from 2,2′-(7-((1-((15S,16S)-3-(carboxymethyl)-15-(((3S,6S)-6-(((2R,3S)-2-methoxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperidin-4-yl)methyl)-1,4,7-triazonane-1,4-diyl)diacetic acid (10.2 mg), and was isolated as a white, fluffy solid.

ES/MS m/z 1156.6 (M+H)⁺.

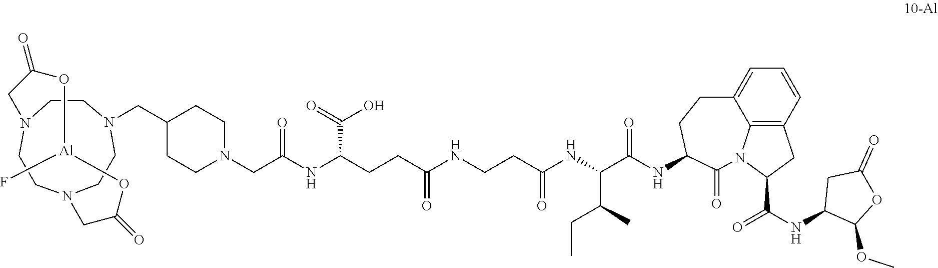

Following the general procedure E, 2,2′-(7-((1-(2-(((S)-1-carboxy-4-((3-(((2S,3S)-1-(((3S,6S)-6-(((2R,3S)-2-methoxy-5-oxotetrahydrofuran-3-yl)carbamoyl)-4-oxo-1,2,3,4,6,7-hexahydroazepino[3,2,1-hi]indol-3-yl)amino)-3-methyl-1-oxopentan-2-yl)amino)-3-oxopropyl)amino)-4-oxobutyl)amino)-2-oxoethyl)piperidin-4-yl)methyl)-1,4,7-triazonane-1,4-diyl)diacetic acid, aluminum fluoride complex (1:1) was detected by HPLC.

ES/MS m/z 1099.5 (M+H)⁺.

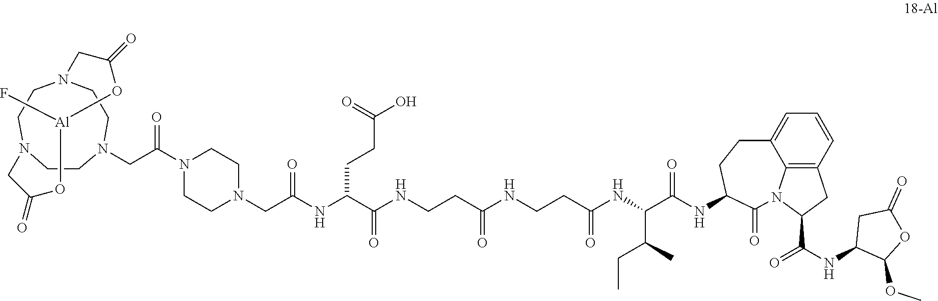

Following the general procedure E, 2,2′-(7-(2-(4-((4R,15S,16S)-4-(2-carboxyethyl)-15-(((3S,6S)-6-(((2R,3S)-2-methoxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperazin-1-yl)-2-oxoethyl)-1,4,7-triazonane-1,4-diyl)diacetic acid, aluminum fluoride complex (1:1) was detected by HPLC.

ES/MS m/z 1199.6 (M+H)⁺.

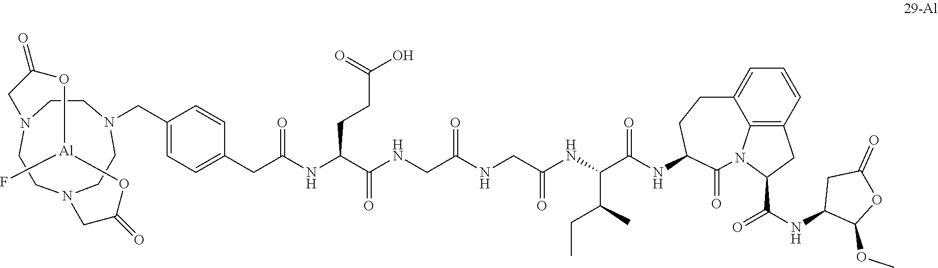

Following the general procedure E, 2,2′-(7-(4-((4S,13S,14S)-4-(2-carboxyethyl)-13-(((3S,6S)-6-(((2R,3S)-2-methoxy-5-oxotetrahydrofuran-3-yl)carbamoyl)-4-oxo-1,2,3,4,6,7-hexahydroazepino[3,2,1-hi]indol-3-yl)carbamoyl)-14-methyl-2,5,8,11-tetraoxo-3,6,9,12-tetraazahexadecyl)benzyl)-1,4,7-triazonane-1,4-diyl)diacetic acid, aluminum fluoride complex (1:1) was detected by HPLC.

ES/MS m/z 1135.4 (M+H)⁺.

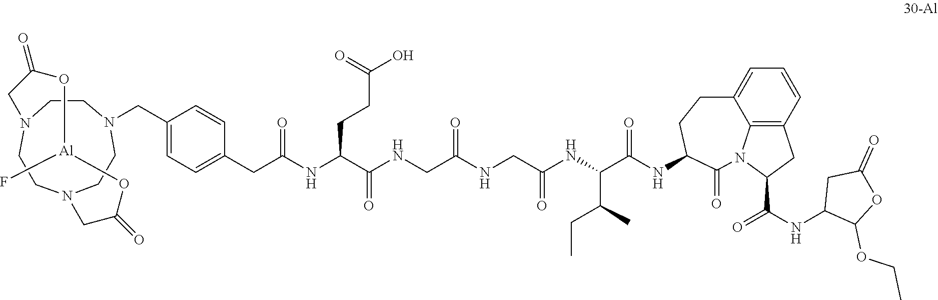

A stirred solution of 2,2′-(7-(4-((4S,13S,14S)-4-(2-carboxyethyl)-13-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-14-methyl-2,5,8,11-tetraoxo-3,6,9,12-tetraazahexadecyl)benzyl)-1,4,7-triazonane-1,4-diyl)diacetic acid, aluminum fluoride complex (1:1) (15.0 mg) in a mixture of 3.0 mL ethanol and 0.75 mL ethyl orthoacetate with ~20 uL TFA was heated to 65° C. overnight. The solution was concentrated, and the residue was redissolved in 10% DMSO in H₂O. The mixture was purified by HPLC (0.1% FA in H₂O to CH₃CN, 0-60%) to afford 2,2′-(7-(4-((4S,13S,14S)-4-(2-carboxyethyl)-13-(((3S,6S)-6-((2-ethoxy-5-oxotetrahydrofuran-3-yl)carbamoyl)-4-oxo-1,2,3,4,6,7-hexahydroazepino[3,2,1-hi]indol-3-yl)carbamoyl)-14-methyl-2,5,8,11-tetraoxo-3,6,9,12-tetraazahexadecyl)benzyl)-1,4,7-triazonane-1,4-diyl)diacetic acid, aluminum fluoride complex (1:1) (5.5 mg) as an off white solid.

ES/MS m/z 1149.5 (M+H)⁺.

### Example 4: Radiosynthesis of Compound ¹⁸F-4-Al

Typical ¹⁸F-4-Al RCY synthesized on an ORA Neptis Perform radiosynthesizer ranges from 42-45% using 20-26 GBq starting activity. Synthesis time is 62±5 minutes with product radiochemical purity>97% and specific activity ranging from 744-894 GBq/pmol (17-21 mCi/μg).

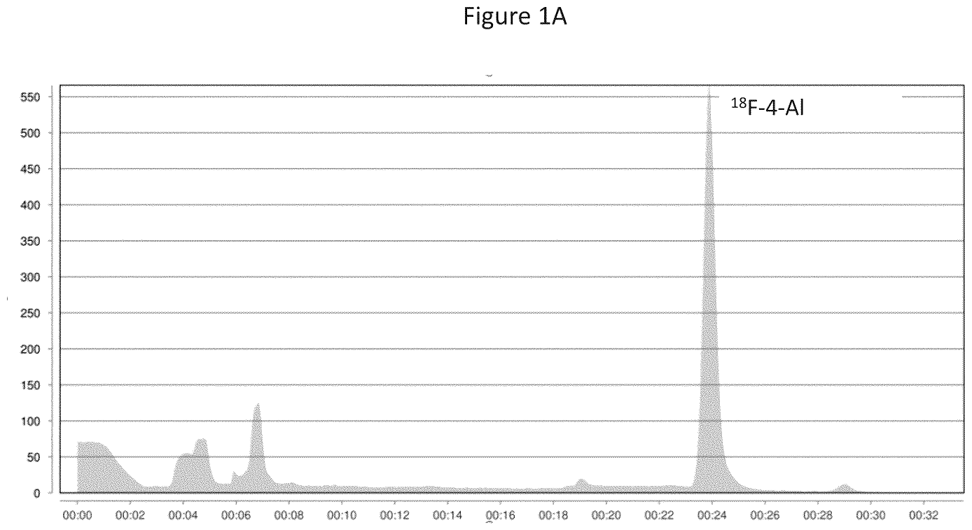

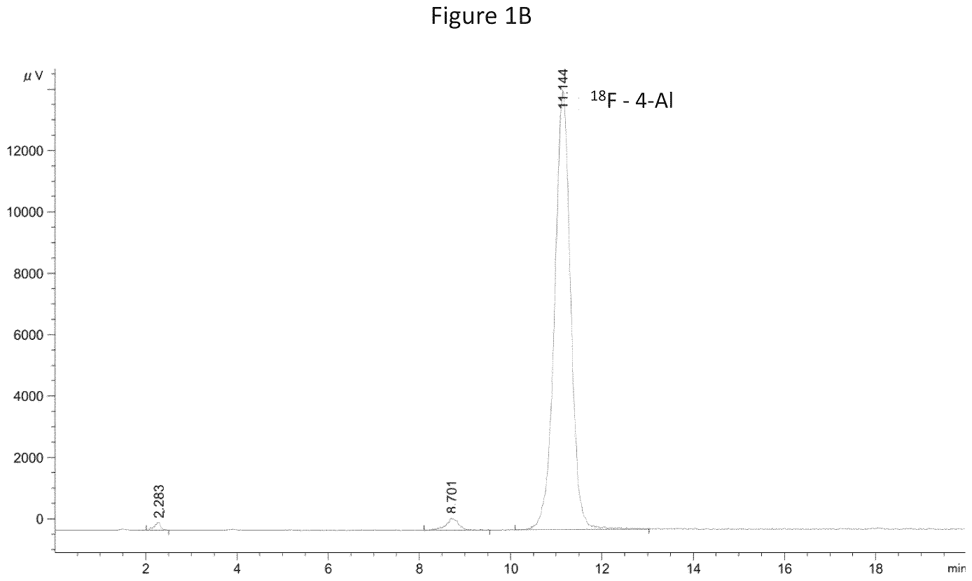

Reaction vessel was preloaded with precursor (0.22 mg), AlCl₃·6H₂O (38.6 μg), acetic acid/sodium acetate buffer (0.2 mL, 1 M, pH=3.5), water (0.19 mL), and acetonitrile (0.8 mL). [¹⁸F]Fluoride was retained on a conditioned Waters Sep-Pak Accell Plus QMA Carbonate Plus Light Cartridge (46 mg sorbent per cartridge, 40 μm particle size, Waters Part No. 186004540) and was then eluted into the reaction vessel using 0.9% saline (0.8 mL). The resulting mixture was kept at 105° C. for 15 minutes and then cooled down to 60° C. 4.5 mL of water was added to the reaction vessel. The diluted reaction mixture was then loaded onto semi-preparative HPLC column (Agilent ZORBAX Eclipse XDB-C18, 5 μm, 9.4×250 mm, Part No. 990967-202) and purified using mobile phase comprising 82% 20 mM ammonium acetate aqueous solution (pH 7) and 18% acetonitrile with flowrate of 4 mL/min. Typical peak collection ranged from 23-25 minutes. (FIG. 1A) The collected fraction was then diluted with 0.5% (w/v) sodium ascorbate aqueous solution (~30 mL) and passed through a conditioned Waters Sep-Pak C18 Plus Light cartridge (130 mg sorbent per cartridge, 55-105 μm, Part No: 023501). Product retained on the cartridge was then washed with sodium ascorbate aqueous solution (0.5%, w/v, ~15 mL) and then eluted with 1 mL ethanol into final product vial containing 6 mL of 0.9% saline and sodium ascorbate (0.5%, w/v). The C18 cartridge was then rinsed with additional 3 mL of 0.9% saline and sodium ascorbate (0.5%, w/v) to afford 10 mL formulated product as 10% v/v ethanol and 90% v/v 0.9% saline containing 0.5% w/v sodium ascorbate. A sample from the product vial was taken out for HPLC analysis. (FIG. 1B)

### Example 5: In Vivo Imaging of ¹⁸F-Granzyme B Tracers in a Matrigel Mouse Model of Active and Pro-Form Granzyme B

This example explores the in vivo imaging activity of exemplary prodrug compounds disclosed herein, which are capable of producing granzyme B-binding compounds in vivo.

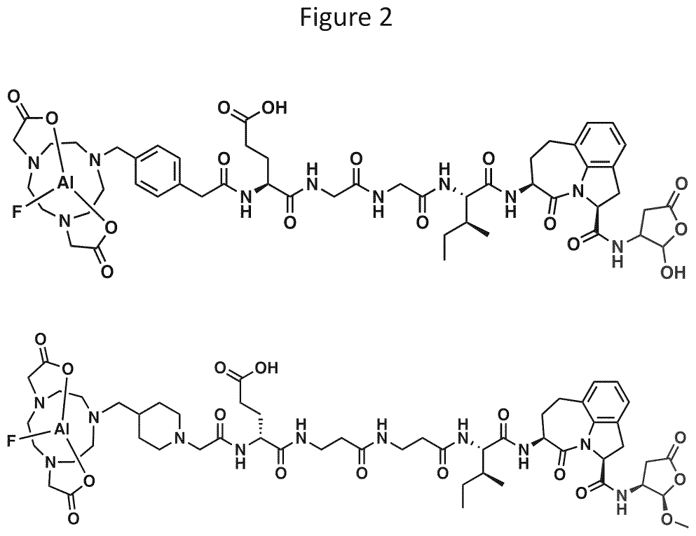

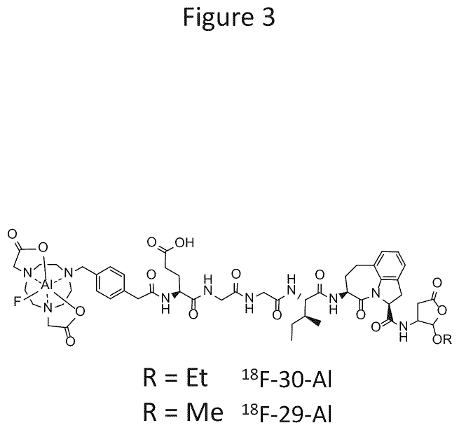

Female nude athymic (5-6 weeks, 15-30 g) mice were purchased from Jackson Laboratories. Both granzyme B (Human lymphocytes, Enzo Life Sciences) and inactivated human pro-form granzyme B (R&D Systems) were also commercially purchased. On the day of the imaging study, each mouse had a cannula inserted into the lateral tail vein (SAI 27 g butterfly with 12 cm tubing, #BF27-01) to allow for intravenous radiotracer administration. Each granzyme B enzyme were then brought up to a final concentration of 0.05 μg/ul using phosphate buffered saline (GE, Hyclone). Matrigel (65 μl, Corning) was mixed with 15 μl of 0.75 μg of each granzyme B enzyme (granzyme B and pro-form granzyme B) within each Eppendorf tube. The mice were then anesthetized with 2.5-3% isoflurane mixed with oxygen. Approximately 60-80 μl of granzyme B/Matrigel or the pro-form granzyme B/Matrigel were then injected using a 28-gauge×0.5-inch insulin syringe (Terumo) to form implants on the right and left shoulder flanks of the mice. Approximately five minutes later, the mice were administered the ¹⁸F-radiotracer via a bolus intravenous injection (approximately 150 μCi in a total volume of 100 μl saline plus an additional ~25 μl saline to flush the catheter line). After the radiotracer injection, the catheter was removed and measured for any remaining residual radioactivity. The mice were then placed back in their cage for recovery. A nanoScan® PET/CT (Mediso, Hungary) was used for micro-PET/CT imaging in which 15-minute static PET scans were conducted 75-minutes post-injection of the radiotracer. Tera-Tomo™ 3D PET iterative reconstruction along with scatter correction were then conducted post-acquisition. A short high-resolution computed tomography (CT) scan was also performed immediately after to allow for anatomical registration. PET signal in the granzyme B implants were quantified by manually drawing regions of interest (ROIs) over the Matrigel implants based on the fused PET/CT images and the corresponding activity values were determined with VivoQuant (Invicro, Massachusetts). All values were represented as % injected dose per gram (% ID/g). Target to background ratios (TBR's) were then calculated by dividing the granzyme B % ID/g value by the pro-form granzyme B % ID/g value. FIG. 2 shows the chemical structures of the active form of Compound 29-Al and Compound 4-Al (an exemplary prodrug). FIG. 3 shows the chemical structure of Compound 29-Al (R=methyl; MeO prodrug) and Compound 30-Al (R=ethyl, EtO prodrug). The pro-form of the compounds showed lower % ID/g and TBR as compared with the corresponding active forms.

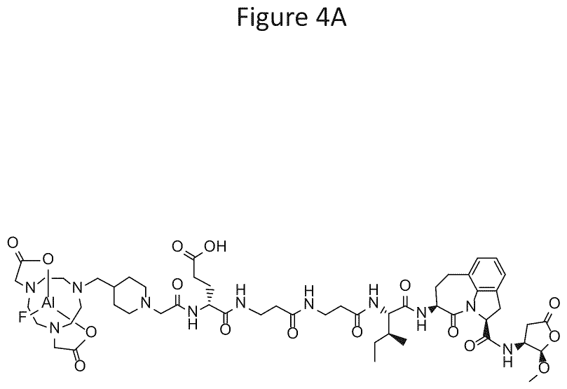

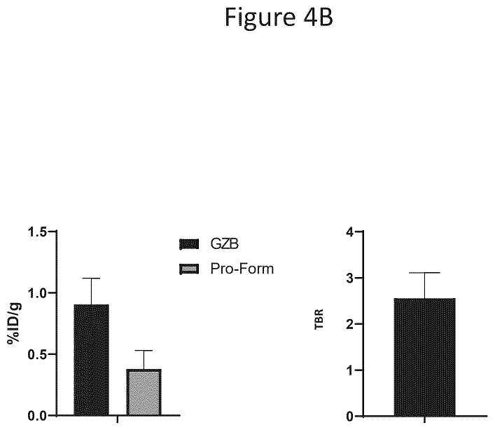

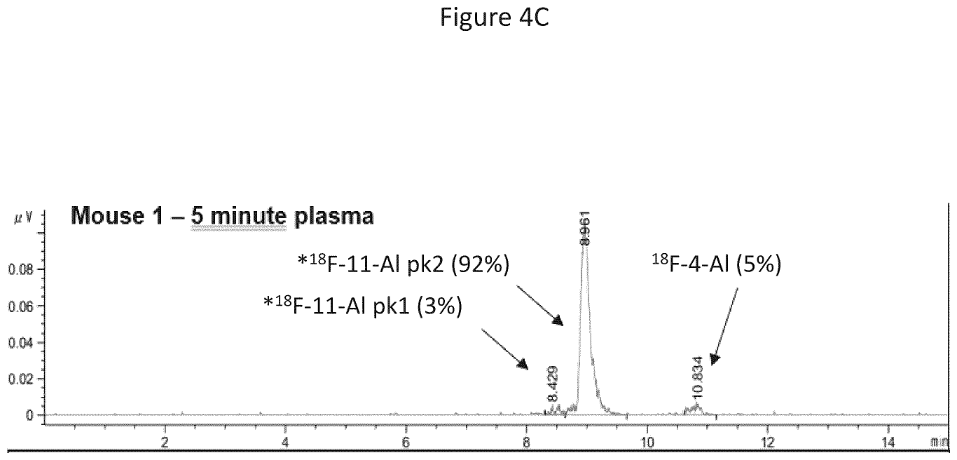

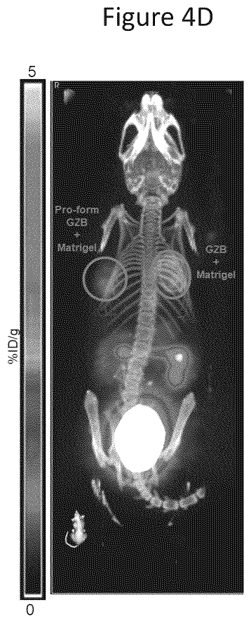

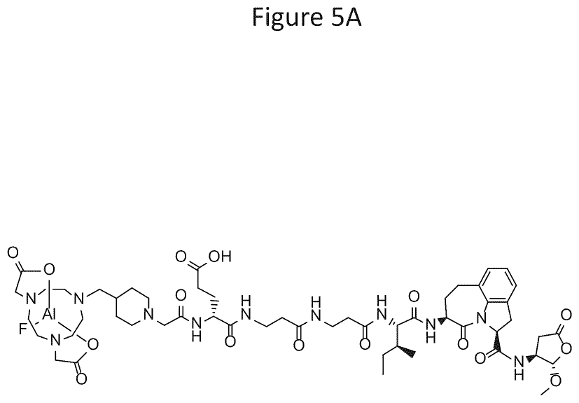

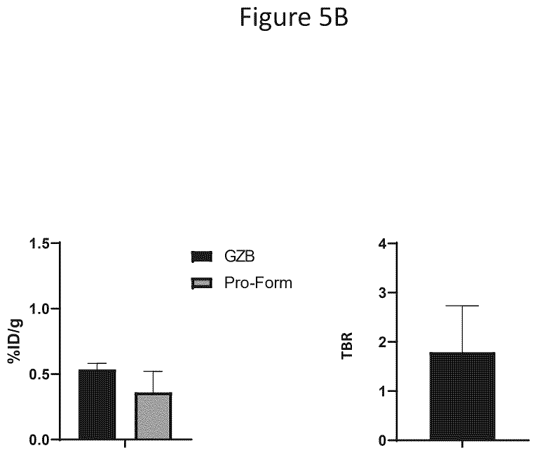

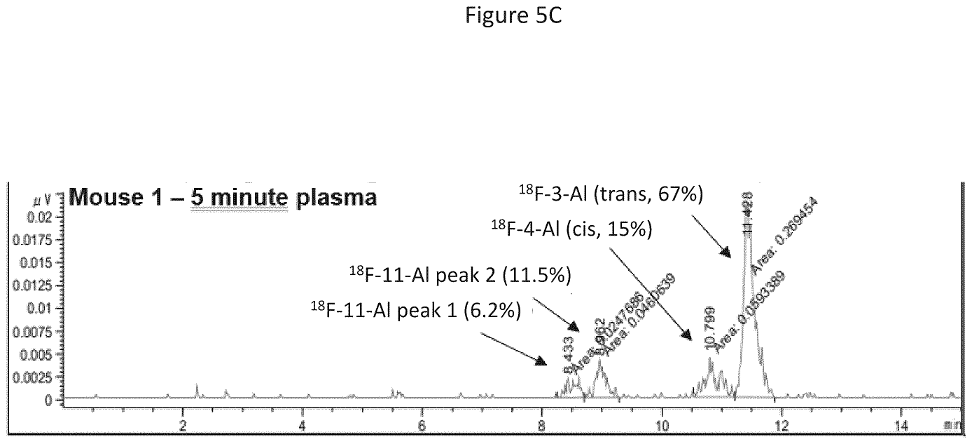

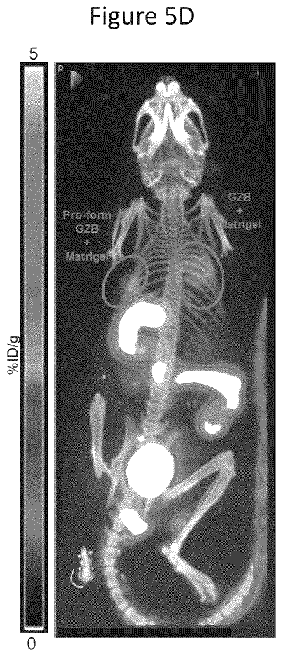

Two cis/trans pairs of exemplary prodrug compounds, Compound ¹⁸F-4-Al (cis-)/Compound ¹⁸F-3-Al (trans-), and Compound ¹⁸F-6-Al (cis-)/Compound ¹⁸F-7-Al (trans-), were examined in this example. The cis-isomer of Compound ¹⁸F-4-Al showed rapid conversion to the active form and good differentiation in Matrigel. FIGS. 4A-4D. Trans isomer of Compound ¹⁸F-3-Al showed slow conversion to the active form and poor differentiation in Matrigel. FIGS. 5A-5D.

### Example 6: Metabolism of Exemplary Compounds

In vivo conversion of prodrugs was monitored by radioactive metabolite screening studies performed by injection of [¹⁸F] granzyme B tracers in male CD-1 mice followed by analysis of extracted blood samples by radiometric HPLC. Results are reported as percent active form present in blood (Table 11).

Briefly, male CD-1 mice were anesthetized prior to dosing with approximately 250 μCi [¹⁸F] granzyme B tracer via tail vein injection (n=3 mice/timepoint). For blood collection, mice were sacrificed following cardiac exsanguination with a syringe pre-rinsed with sodium heparin at 5 and 15 minutes post-injection and samples were prepared for analysis via radio-HPLC.

Blood samples were centrifuged at 1500 RCF for 5 minutes to allow for separation of plasma. Plasma samples were mixed with two volumes of methanol, vortexed for 30 seconds, and then centrifuged at 1500 RCF for 2 minutes. The supernatant was separated from the pellet and diluted 1:4 with PBS buffer pH 7.4 or water prior to analysis by HPLC.

Samples were then analyzed on an Agilent 1290 series UPLC UV coupled with a BGO coincidence detector. The HPLC method utilized 2) Phenomenex Monolithic C18 (100×4.6 mm); 100-200 μL injection volume; flow-rate: 1.2 mL/min; solvent for A: 20 mM ammonium acetate in water; solvent for B: 100% methanol; gradient: initial hold at 5% B hold for 1 min, 5% to 40% in 7 min, hold 40% B for 2 min, increase from 40% to 95% B in 1 min, hold 95% B for 2 min, and then return to 5% B to reequilibrate.

Table 11 provides the results from this example, reported as percent active form present.

TABLE 11
Summary of in-vivo conversion of [¹⁸F]
granzyme B prodrugs in male CD-1 mice.

| | Prodrug | Alkyl | 5 min (% active | 15 min (% active |
|---|---|---|---|---|
| Compound | isomer | ether | form in blood) | form in blood) |
| [¹⁸F] 3-Al | trans | MeO | 13 | 17 |
| [¹⁸F] 4-Al | cis | MeO | 92 | 94 |
| [¹⁸F] 5-Al | cis | EtO | 98 | 100 |
| [¹⁸F] 6-Al | trans | MeO | 11 | 16 |
| [¹⁸F] 7-Al | cis | MeO | 69 | 78 |
| [¹⁸F] 8-Al | cis | EtO | 86 | 100 |
| [¹⁸F] 9-Al | cis | MeO | 90 | 100 |

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Further, from the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A compound, or a pharmaceutically acceptable salt thereof, wherein the compound is of formula (I): 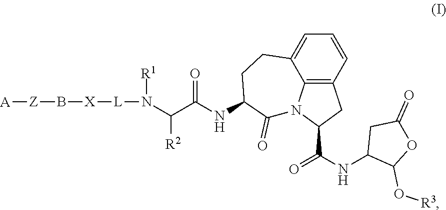
wherein:
A is a chelating moiety;
B is selected from the group consisting of aryl, heteroaryl, cycloalkyl, and heterocyclyl; optionally wherein B is a 6-membered ring;
X is selected from the group consisting of —CH₂C(NH)—, —CH₂C(O)—, —CH₂C(S)—, —NHC(NH)—, —NHC(O)—, —NHC(S)—, —OC(NH)—, —OC(O)—, and —OC(S)—, optionally wherein X is —CH₂C(O)— or —NHC(S)—;
Z is —CH₂—, —CH₂C(NH)—, —CH₂C(O)—, —CH₂C(S)—, —NHC(NH)—, —NHC(O)—, —NHC(S)—, —OC(NH)—, —OC(O)—, and —OC(S)—; optionally wherein Z is —CH₂— or —CH₂C(O)—;
L is a peptide linker having 1-6 amino acid residues, inclusive;
R¹ is H or C₁₋₆ alkyl, optionally wherein R¹ is H or methyl;
R² is C₁₋₆ alkyl or C₃₋₆ cycloalkyl; and
R³ is C₁₋₆ alkyl.

2. The compound, or the pharmaceutically acceptable salt thereof, of claim 1, wherein the compound is of formula (Ia): 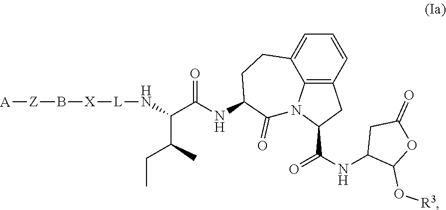
and wherein A, B, X, Z, L, and R³ each are defined in claim 1.

3. The compound, or the pharmaceutically acceptable salt thereof, of claim 1, wherein the compound is of formula (Ib): 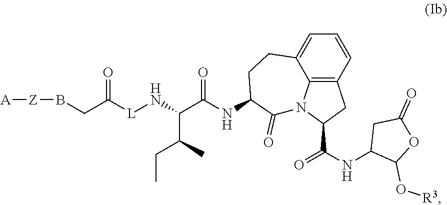
and wherein A, B, Z, L, and R³ each are defined in claim 1.

4. The compound, or the pharmaceutically acceptable salt thereof, of any one of claims 1-3, wherein the chelating moiety A is 1,4,7-triazacyclononane-N, N′, N″-triacetic acid (NOTA) or 1,4,7-triazacyclononane-4,7-diyl diacetic acid (NODA).

5. The compound, or a pharmaceutically acceptable salt thereof, of any one of claims 1-4, wherein L has 1-3 amino acid residues, inclusive.

6. The compound, or a pharmaceutically acceptable salt thereof, of any one of claim 1-4, wherein L has 3-6 amino acid residues, inclusive.

7. The compound, or a pharmaceutically acceptable salt thereof, of any one of claims 1-6, wherein L has an amino acid sequence selected from the group consisting of:
Gly,
Gly-Gly,
Gln-Gly,
Glu,
Glu-Gly,
Glu-Gly-Gly,
Glu-βAla-βAla,
_{D}Glu,
_{D}Glu-βAla-βAla,
_{D}Glu-Gly-Gly,
_{D}Glu-AEA,
_{D}Glu-AEEA-AEEA,
_{D}Glu-_{D}Glu-AEA,
_{D}Glu-_{D}Glu-βAla-βAla,
γGlu,
γGlu-βAla,
_{D}γGlu,
Lys-Gly,
Arg-Gly,
N-Acid-βAla-βAla,
βAla-N-Acid-βAla,
βAla-Glu-Gly-Gly,
βAla-_{D}Glu-βAla, and
Diacid-βAla-βAla.

8. The compound, or the pharmaceutically acceptable salt thereof, of any one of claims 1-7, wherein the compound is of formula (Ic): 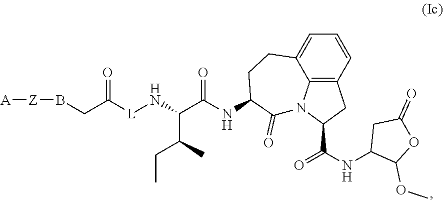
and wherein A, B, Z, and L each are defined in any one of claims 1-7.

9. The compound, or the pharmaceutically acceptable salt thereof, of claim 8, wherein the compound is of formula (Ic-A): 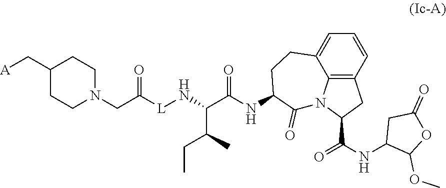
wherein A and L are each defined in claim 8.

10. The compound, or the pharmaceutically acceptable salt thereof, of claim 9, wherein the compound is of formula (Ic-Aa): 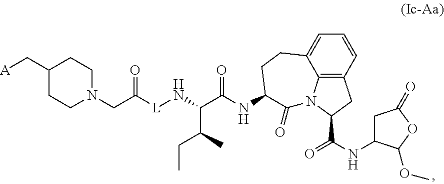
optionally wherein the compound is of formula (Ic-Ab): 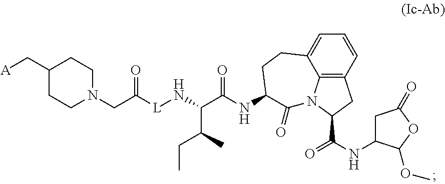
and wherein A and L are each defined in claim 9.

11. The compound, or the pharmaceutically acceptable salt thereof, of claim 9, wherein the compound is selected from the group consisting of Compounds 1-17.

12. The compound, or the pharmaceutically acceptable salt thereof, of claim 8, wherein the compound is of formula (Ic-B): 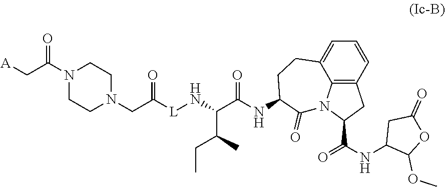
wherein A and L are each defined in claim 8.

13. The compound, or the pharmaceutically acceptable salt thereof, of claim 12, wherein the compound is of formula (Ic-Ba): 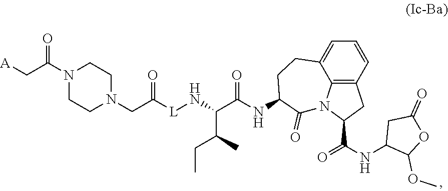
optionally wherein the compound is of formula (Ic-Bb): 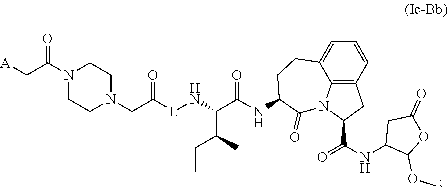
and wherein A and L are each defined in claim 12.

14. The compound, or the pharmaceutically acceptable salt thereof, of claim 12, wherein A is NODA; optionally wherein the compound is selected from the group consisting of Compounds 18-28.

15. The compound, or the pharmaceutically acceptable salt thereof, of claim 8, wherein the compound is of formula (Ic-C): 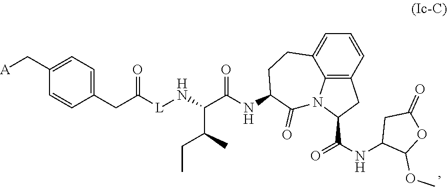
and wherein L is defined in claim 8.

16. The compound, or the pharmaceutically acceptable salt thereof, of claim 15, wherein the compound is of formula (Ic-Ca): 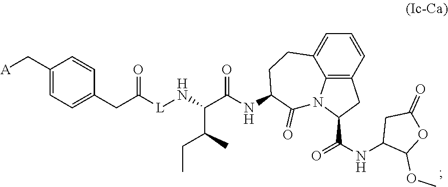
optionally wherein the compound is of formula (Ic-Cb): 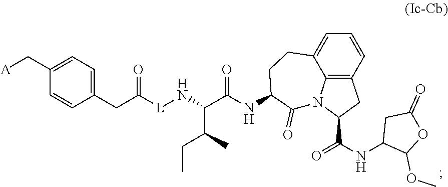
and wherein L is defined in claim 15.

17. The compound, or a pharmaceutically acceptable salt thereof, of claim 15, wherein A is NODA; optionally wherein the compound is selected from the group consisting of Compounds 29-46:

18. The compound, or the pharmaceutically acceptable salt thereof, of any one of claims 1-7, wherein the compound is of formula (Id): 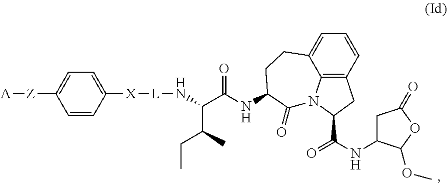
and wherein A, Z, X, and L each are defined in claim 1.

19. The compound, or the pharmaceutically acceptable salt thereof, of claim 18, wherein the compound is of formula (Id-A): 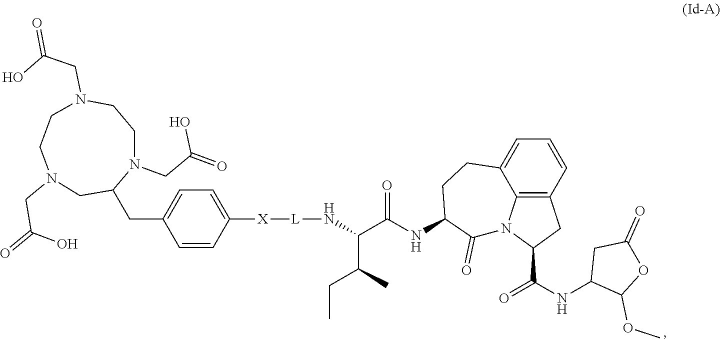
and wherein X and L are defined in claim 18.

20. The compound, or the pharmaceutically acceptable salt thereof, of claim 19, wherein the compound is of formula (Id-Aa): 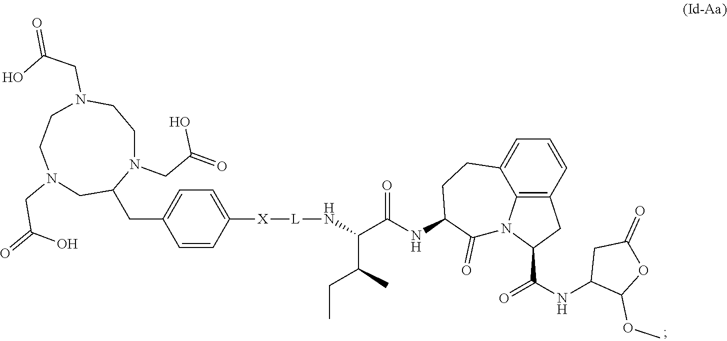
optionally wherein the compound is of formula (Id-Ab): 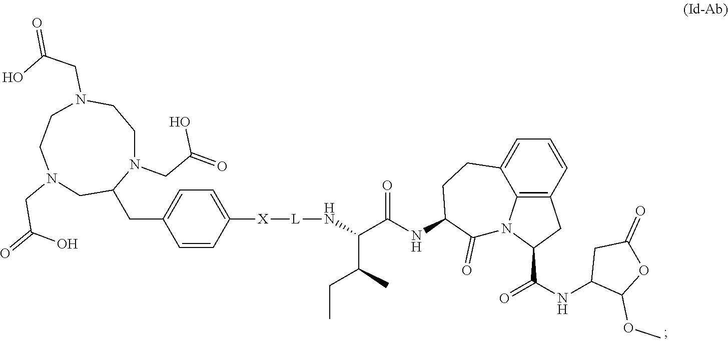
and wherein X and L is defined in claim 18.

21. The compound, or the pharmaceutically acceptable salt thereof, of claim 19, wherein the compound is selected from the group consisting of Compounds 47-49:

22. A compound, or a pharmaceutically acceptable salt thereof, wherein the compound is of formula (II): 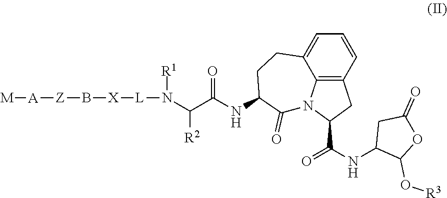
wherein:
M is a metal or a metal linked to a radioisotope;
A is a chelating moiety chelating the metal;
B is selected from the group consisting of aryl, heteroaryl, cycloalkyl, and heterocyclyl; optionally wherein B is a 6-membered ring;
X is selected from the group consisting of —CH₂C(NH)—, —CH₂C(O)—, —CH₂C(S)—, —NHC(NH)—, —NHC(O)—, —NHC(S)—, —OC(NH)—, —OC(O)—, and —OC(S)—, optionally wherein X is —CH₂C(O)— or —NHC(S)—;
Z is —CH₂—, —CH₂C(NH)—, —CH₂C(O)—, —CH₂C(S)—, —NHC(NH)—, —NHC(O)—, —NHC(S)—, —OC(NH)—, —OC(O)—, and —OC(S)—; optionally wherein Z is —CH₂— or —CH₂C(O)—;
L is a peptide linker having 1-6 amino acid residues, inclusive;
R¹ is H or C₁₋₆ alkyl, optionally wherein R¹ is H or methyl;
R² is C₁₋₆ alkyl or C₃₋₆ cycloalkyl; and
R³ is C₁₋₆ alkyl.

23. The compound, or the pharmaceutically acceptable salt thereof, of claim 22, wherein the compound is of formula (IIa): 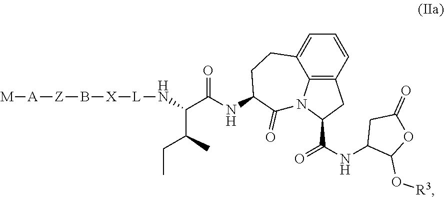
and wherein M, A, B, X, Z, L, and R³ are each as defined in claim 22.

24. The compound, or the pharmaceutically acceptable salt thereof, of claim 22, wherein the compound is of formula (IIb): 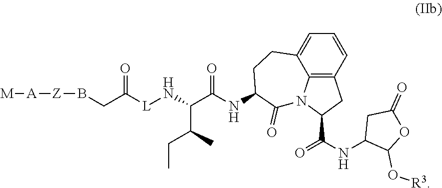
and wherein M, A, B, Z, L, and R³ are each as defined in claim 22.

25. The compound, or the pharmaceutically acceptable salt thereof, of any one of claims 22-24, wherein the chelating moiety A is 1,4,7-triazacyclononane-N,N′,N″-triacetic acid (NOTA) or 1,4,7-triazacyclononane-4,7-diyl diacetic acid (NODA).

26. The compound, or the pharmaceutically acceptable salt thereof, of any one of claims 22-25, wherein L has 1-3 amino acid residues, inclusive.

27. The compound, or the pharmaceutically acceptable salt thereof, of any one of claims 22-25, wherein L has 3-6 amino acid residues, inclusive.

28. The compound, or the pharmaceutically acceptable salt thereof, of any one of claims 22-27, wherein L has an amino acid sequence selected from the group consisting of:
Gly,
Gly-Gly,
Gln-Gly,
Glu,
Glu-Gly,
Glu-Gly-Gly,
Glu-βAla-βAla,
_{D}Glu,
_{D}Glu-βAla-βAla,
_{D}Glu-Gly-Gly,
_{D}Glu-AEA,
_{D}Glu-AEEA-AEEA,
_{D}Glu-_{D}Glu-AEA,
_{D}Glu-_{D}Glu-βAla-βAla,
γGlu,
γGlu-βAla,
_{D}γGlu,
Lys-Gly,
Arg-Gly,
N-Acid-βAla-βAla,
βAla-N-Acid-βAla,
βAla-Glu-Gly-Gly,
βAla-_{D}Glu-βAla, and
Diacid-βAla-βAla.

29. The compound, or the pharmaceutically acceptable salt thereof, of any one of claims 22-28, wherein the metal linked to a radioisotope is Al; optionally wherein the radioisotope is ¹⁸F.

30. The compound, or the pharmaceutically acceptable salt thereof, of any one of claims 22-29, wherein the compound is of formula (IIc): 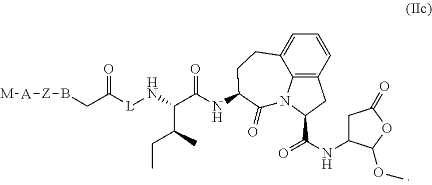
and wherein M, A, B, Z and L are each as defined in any one of claims 22-29.

31. The compound, or the pharmaceutically acceptable salt thereof, of claim 30, wherein the compound is of formula (IIc-A): 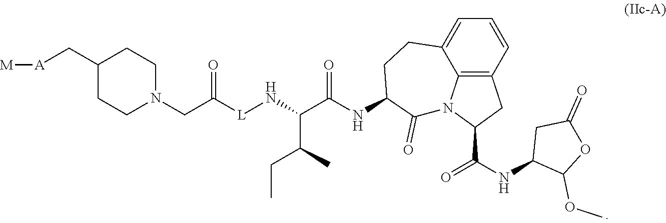
and wherein M, A, and L are each defined in claim 30.

32. The compound, or the pharmaceutically acceptable salt thereof, of claim 31, wherein the compound is of formula (IIc-Aa): 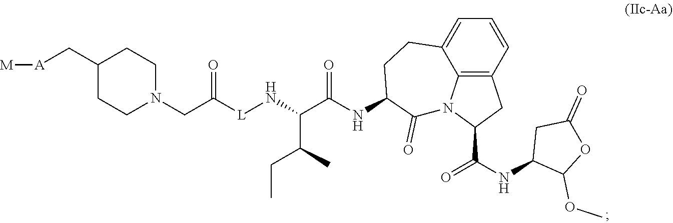
optionally wherein the compound is of formula (IIc-Ab): 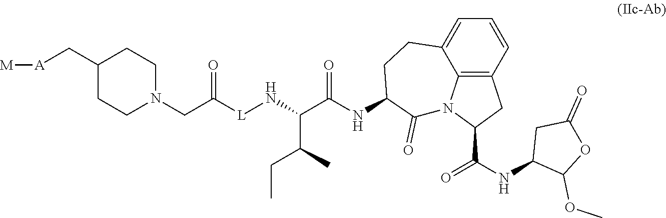
and wherein M, A, and L are each as defined in claim 29.

33. The compound or the pharmaceutically acceptable salt thereof, of claim 31 or claim 32, wherein A is NODA.

34. The compound or the pharmaceutically acceptable salt thereof, of claim 33, wherein the compound is selected from the group consisting of Compound 1-Al to 17-Al.

35. The compound, or the pharmaceutically acceptable salt thereof, of claim 30, wherein the compound is of formula (IIc-B): 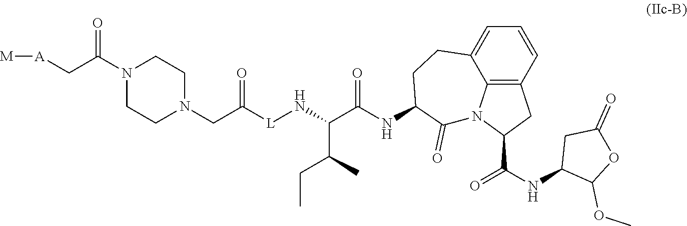
wherein M, A and L are each defined in claim 30.

36. The compound, or the pharmaceutically acceptable salt thereof, of claim 35, wherein the compound is of formula (IIc-Ba): 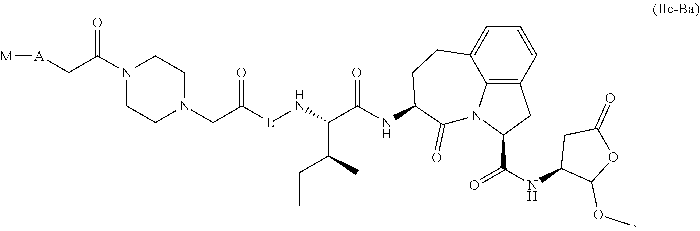
optionally wherein the compound is of formula (Ic-Bb): 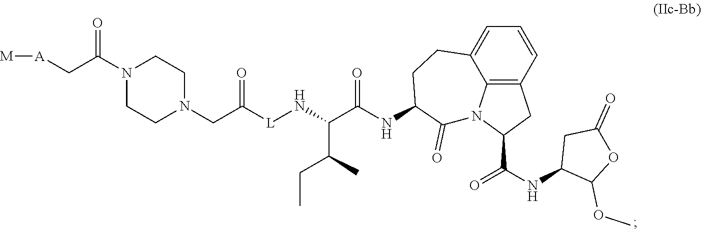
and wherein M, A and L are each defined in claim 35.

37. The compound, or the pharmaceutically acceptable salt thereof, of any one of claims 22-29, wherein the compound is of formula (IIc-C): 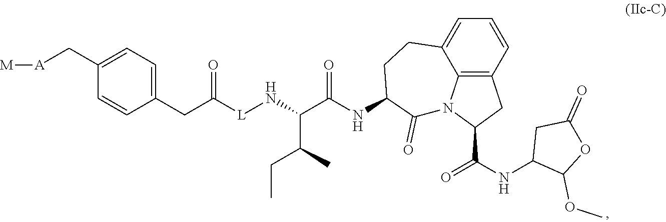
and wherein A and L is defined as in any one of claims 22-29.

38. The compound, or the pharmaceutically acceptable salt thereof, of claim 37, wherein the compound is of formula (IIc-Ca): 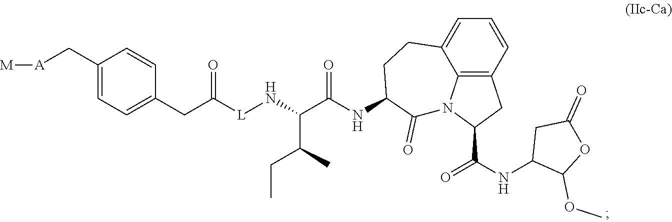
optionally wherein the compound is of formula (IIc-Cb): 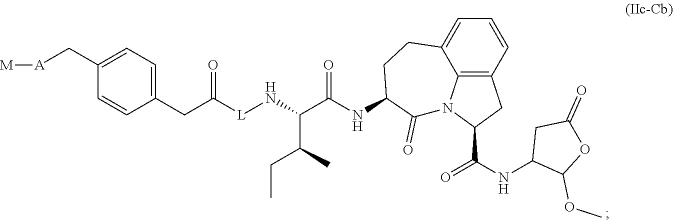
and wherein A and L are each defined in claim 39.

39. The compound or the pharmaceutically acceptable salt thereof of claim 38, wherein A is NODA; optionally wherein the compound is selected from the group consisting of Compounds 18-28.

40. The compound, or the pharmaceutically acceptable salt thereof, of any one of claims 22-28, wherein the metal is Ga, which optionally is ⁶⁸Ga.

41. The compound, or the pharmaceutically acceptable salt thereof, of any one of claims 22-28 or 40, wherein the compound is of formula (IId): 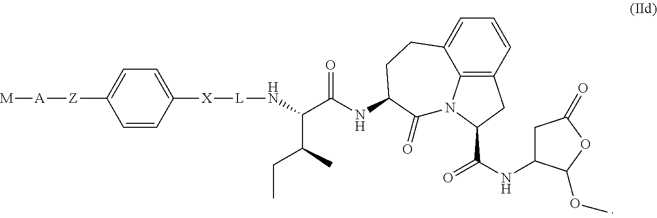
and wherein M, A, Z, X, and L are each defined as in any one of claims 22-28 or 40.

42. The compound, or the pharmaceutically acceptable salt thereof, of claim 41, wherein the compound is of formula (IId-A): 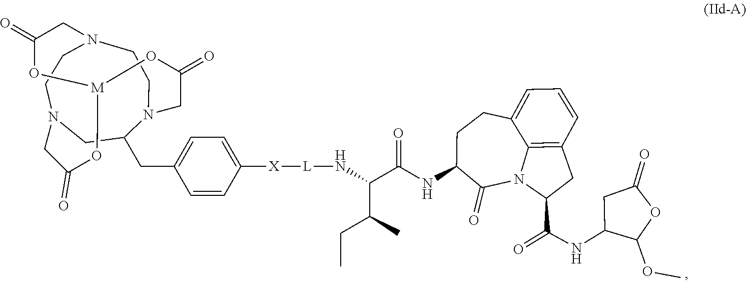
and wherein M, X, and L are each defined as in claim 41.

43. The compound, or the pharmaceutically acceptable salt thereof, of claim 42, wherein the compound is of formula (IId-Aa): 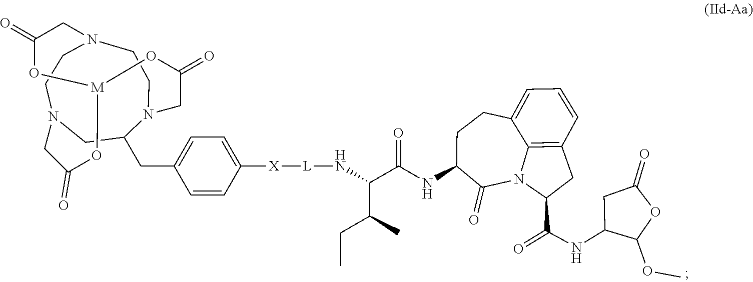
optionally wherein the compound is of formula (IId-Ab): 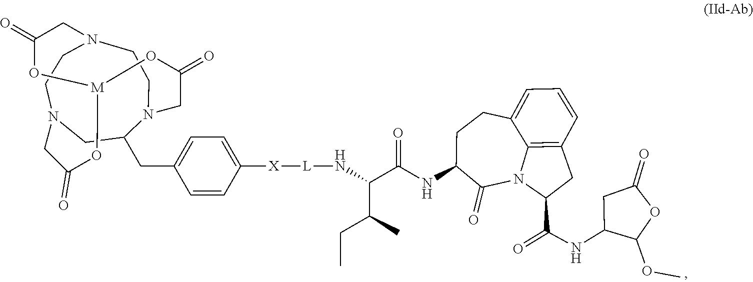
and wherein M, X, and L are each defined in claim 42.

44. The compound, or the pharmaceutically acceptable salt thereof, of claim 42, wherein the compound is selected from the group consisting of Compounds 47-49.

45. A pharmaceutical composition comprising a compound of any one of claims 1-44, a stereoisomer thereof, or a pharmaceutically acceptable salt thereof, and a pharmaceutically acceptable carrier.

46. A method of treating an immunoregulatory abnormality in a subject, the method comprising administering to a subject in need thereof an effective amount of the compound of any one of claims 22-44, and optionally one or more additional therapeutic agents for treating the immunoregulatory abnormality.

47. The method of claim 46, wherein the one or more additional therapeutic agents are selected from the group consisting of anti-inflammatory agents, steroids, immunotherapy agents, chemotherapeutic agents, and therapeutic antibodies.

48. The method of claim 47, wherein the immunoregulatory abnormality is selected from the group consisting of an autoimmune disorder, an inflammatory disorder, a skin disorder, a cancer, and a cardiovascular disorder.

49. The method of claim 48, wherein the immunoregulatory abnormality is a cancer.

50. A method of imaging granzyme B in a tissue, the method comprising:
(i) contacting the compound or the pharmaceutically acceptable salt thereof, of any one of claims 22-44, with a tissue suspected of comprising granzyme B, and
(ii) imaging the tissue based on radioisotope signals released from the compound or the pharmaceutically acceptable salt thereof,
(iii) optionally wherein the radioisotope contained in the compound or the pharmaceutically acceptable salt thereof, is ¹⁸F or ⁶⁸Ga.

51. The method of claim 50, wherein the contacting step is performed by administering the compound or the pharmaceutically acceptable salt thereof to a subject in need thereof.

52. The method of claim 51, wherein the subject is on a treatment of an immunoregulatory abnormality.

53. The method of claim 52, wherein the immunoregulatory abnormality is selected from the group consisting of an autoimmune disorder, an inflammatory disorder, a skin disorder, a cancer, and a cardiovascular disorder, optionally wherein the immunoregulatory abnormality is a cancer.

54. The method of claim 53, wherein treatment comprises one or more additional therapeutic agents, which are selected from the group consisting of anti-inflammatory agents, steroids, immunotherapy agents, chemotherapeutic agents, and therapeutic antibodies.

55. The method of any one of claims 46-54, further comprising monitoring an immune response in the subject based on the imaging of granzyme B.
